(19) United States
(12) Patent Application Publication
(10) Pub. No.: US 2026/0047775 A1
(43) Pub. Date: Feb. 19, 2026

(54) GAS COLLECTOR

(71) Applicant: Fisher & Paykel Healthcare Limited, Auckland (NZ)

(72) Inventors: William Herbert STURME, Auckland (NZ); David Petrus BRUWER, Auckland (NZ); Maxim Darius TOURANI RAD, Auckland (NZ); Taylor James EDWARDS, Auckland (NZ); Catherine Rosamond BRIDGMAN, Auckland (NZ); Samuel Thomas STRUTHERS, Auckland (NZ); Isabela MONTEIRO DE ANDRADE, Auckland (NZ); Max William Anstiss HAYMAN, Auckland (NZ)

(21) Appl. No.: 19/103,970

(22) PCT Filed: Aug. 18, 2023
(86) PCT No.: PCT/IB2023/058268

§ 371 Date: Feb. 14, 2025

Related U.S. Application Data
(60) Provisional application No. 63/371,964, filed on Aug. 19, 2022.

(51) Int. Cl. A61B 5/097; A61M 16/06; A61M 16/08

(52) U.S. Cl. CPC A61B 5/097; A61M 16/0672; A61M 16/085; A61M 2205/3334

(57) ABSTRACT

The present invention is directed to a gas collector for collecting gases at a patient. The gas collector may include an interface configured to form at least one channel at an upper lip of the patient. The channel may be in fluid communication with the patient's nose and mouth regions. The interface may include one or more spacers configured to contact the patient's face so as to space a channel wall portion from the patient's face.

### TECHNICAL FIELD

This disclosure relates to a gas collector for collecting gases at a patient. The gas collector is suitable for use in applications requiring the collection and monitoring of gases exhaled by a patient from their nasal passages and/or oral passage whilst providing gas flow to a patient. It will be convenient to describe the gas collector in relation to that exemplary but non-limiting application.

### BACKGROUND OF THE INVENTION

Medical procedures may involve procedural sedation or general anaesthesia, collectively known as anaesthetic procedures. In procedural sedation, a patient may have diminished respiratory function or risk thereof due to the anaesthetic agents and may become apnoeic. In general anaesthesia, patients will likely require mechanical ventilation if they become apnoeic. Pre-oxygenation of the patient prior to administration of anaesthetic agents is encouraged. During pre-oxygenation, also known as de-nitrogenation, the patient is provided with oxygen to saturate their body's stores, including blood and lungs, to delay or avoid oxygen desaturation when the patient has diminished respiratory function or is apnoeic. Pre-oxygenation also reduces the patient's blood and lung carbon dioxide levels, and nitrogen levels in the lungs due to replacement by oxygen.

Nasal High Flow (NHF) can be used in anaesthetic procedures to pre-oxygenate a patient, maintain adequate oxygenation and prevent desaturation, and in general anaesthesia when the patient becomes apnoeic, extend safe apnoea time during anaesthesia. NHF utilises a non-sealing patient interface such as a nasal cannula to deliver breathable gases, for example oxygen or oxygen enriched air or air, to a patient. NHF may also be used in other environments, such as wards, emergency departments and intensive care units (ICUs).

It is beneficial to monitor gases at the patient when they are receiving respiratory support. Such monitoring provides useful feedback to clinicians. For example, monitoring exhaled gases during the pre-oxygenation phase can be used to determine whether the patient has reached a desired end expiratory O₂ level which indicates pre-oxygenation is adequate and/or that the pre-oxygenation phase could be complete. Exhaled gas monitoring can be used to detect a change in patient condition, for example, a spontaneously breathing patient becoming apnoeic after being anaesthetised or when experiencing a blocked airway. Clinicians can provide better care by making decisions as a result of patient gas monitoring.

Unfortunately, monitoring of a target gas at a patient while providing a gas flow to the patient can affect the sampling results because the provided gas flow may taint the actual value (e.g. concentration) of the target gas. For example, exhaled CO₂ (carbon dioxide) may be diluted by the provided gases or end tidal O₂ may be enhanced by a higher concentration of O₂ in the provided gas flow. NHF exacerbates this problem due to the high gas flow rates involved.

Difficulties also arise in monitoring exhaled gases due to the variability of the exhalation pathways, that is, via the patient's nose, mouth or nose and mouth. A patient can alternate between these pathways during a period of monitoring. How a patient breathes can affect the gas sampling results as an inadequate amount of exhaled gases may be collected from, say, the nose when the patient is predominantly breathing out of the mouth.

It would be desirable to provide a gas collector for collecting gases at the nasal passages and oral passage of a patient, that ameliorates or overcomes one or more disadvantages or inconveniences of known gas collectors, in particular but not limited to NHF applications. Moreover, it would be desirable to provide a gas collector that enables consistent sampling of the exhaled gases regardless of variability in the exhalation pathways.

The above discussion of the background to the disclosure is intended to facilitate an understanding of the disclosure. However, it is to be appreciated that the discussion is not an acknowledgement or admission that any aspect of the discussion was part of the common general knowledge of a skilled addressee as at the priority date of this application.

### SUMMARY OF THE INVENTION

One aspect of the invention provides a gas collector for collecting gases at a patient, the gas collector including: an interface configured to form at least one channel at an upper lip of the patient, the channel being in fluid communication with the patient's nose and mouth regions, wherein the interface includes one or more spacers configured to contact the patient's face so as to space a channel wall portion from the patient's face.

In one or more embodiments, the interface further includes the channel wall portion.

In one or more embodiments, the interface is configured to be attached to or integral with a nasal cannula.

In one or more embodiments, the nasal cannula at least partially forms the channel wall portion.

In one or more embodiments, the nasal cannula includes nasal prongs for delivering gas to the patient's nasal passages, the gas collector further including one or more openings formed through the channel wall portion for receive the nasal prongs.

In one or more embodiments, the channel is partly formed by the patient's upper lip.

In one or more embodiments, the spacers are disposed to contact the patient's face on either side of the patient's upper lip.

In one or more embodiments, in use, the spacers extend from the channel wall portion to the patient's face.

In one or more embodiments, the spacers are integral with the channel wall portion.

In one or more embodiments, the spacers have a thickness greater than the channel wall portion.

In one or more embodiments, the spacers are more rigid that the channel wall portion.

In one or more embodiments, the spacers have a greater Young's modulus than the channel wall portion.

In one or more embodiments, the channel wall portion is configured to enable it to flex when in contact with the patient's face.

In one or more embodiments, the gas collector further includes an interface forming a gas collection area and including a first and second gas collecting inlets in fluid communication with the gas collection area.

In one or more embodiments, the gas collector further includes a mouth engagement portion configured to project into the patient's mouth so as to maintain an open passage between the patient's mouth and the second gas collecting inlet.

In one or more embodiments, the mouth engagement portion includes a lower end projecting into the patient's mouth, the lower end of the mouth engagement portion being shaped to maintain an open passage between the patient's mouth and the second gas collecting inlet.

In one or more embodiments, the lower end of the mouth engagement portion has an inner surface which includes edge portions located, when in use, at the sides of the patient's mouth, and a central portion located, when in use, at the centre of the patient's mouth and spaced further from the patient's upper lip than the edge portions.

In one or more embodiments, the inner surface of a first end of the mouth engagement portion has a C, U or V shaped profile.

In one or more embodiments, the channel wall portion includes an upper end extending outwardly from the patient's face when in use.

In one or more embodiments, the upper end of the channel wall curves outwardly from the patient's face when in use.

Another aspect of the invention provides a gas collector for collecting gases at a patient, the gas collector including an interface is configured to form a channel at the patient's upper lip, the channel having open ends respectively in fluid communication with the patient's nasal passages and oral passage, the channel providing a volume for gathering gases to be analysed.

In one or more embodiments, the gas collector further includes one or more gas collecting conduits, each having a gas collecting inlet, configured to deliver gases from the channel to a localised gas collector area, and an outlet for providing gathered gases from the localised gas collector area to a gas analyser.

In one or more embodiments, the channel is partly formed by the patient's upper lip.

In one or more embodiments, the gas collector further includes a gas flow diverter configured to funnel at least some of the gases exhaled by a patient to one or more of the gas sampling inlets.

In one or more embodiments, the interface includes one or more spacers for contacting the patient's face to space a channel wall portion from the patient's face, and wherein the gas flow diverter is integrated with the channel wall portion.

In one or more embodiments, the sampler body includes one or more spacers for contacting the patient's face to space a channel wall portion from the patient's face, and wherein the gas flow diverter is formed separately from the channel wall portion.

In one or more embodiments, one of more of the gas sampling inlets form nasal gas sampling inlets located proximate the patient's nasal passages.

In one or more embodiments, the one or more nasal gas sampling inlets are formed in the channel wall portion.

In one or more embodiments, the one or more nasal gas sampling inlets are formed in the spacers.

In one or more embodiments, one or the gas sampling inlets forms an oral gas sampling inlet located proximate the patient's oral passage.

In one or more embodiments, the oral gas sampling inlet is formed in the channel wall portion.

In some embodiments, the gas collector may further include an outlet for providing gathered gases to a gas analyser via a conduit, the outlet being configured to allow connection to the conduit from one side of the patient's face.

The interface may have a pair of opposing lateral sides. The outlet may define a receiving port having an open outlet end for receiving a portion of the conduit therein. The receiving port may be oriented such that the open outlet end faces towards one of the lateral sides. The lateral sides may be configured to generally extend in a direction that is aligned with an imaginary vertical plane bisecting the face of the patient.

In some embodiments, the outlet may be a single outlet of the gas collector to provide gathered gases to the gas analyser.

In some embodiments, the gas collector may further include a single gas sampling inlet configured to provide gathered gases from the channel to the outlet.

In some embodiments, the gas collector may further include a mounting portion for mounting the gas collector to a nasal cannula, the nasal cannula for delivering breathable gas to a patient, the mounting portion defining a sleeve configured to fit over a portion of the nasal cannula.

In some embodiments, the sleeve generally follows an external contour of the portion of the nasal cannula.

In some embodiments, the sleeve may define one or more slits. The one or more slits may allow insertion of the portion of the nasal cannula therethrough so as to enable the portion of the nasal cannula to be received in the sleeve.

In some embodiments, the sleeve may have a resilient wall so as to allow a width of the one or more slits to be manually adjustable.

Another aspect of the invention provides a gas collector for collecting gases at a patient, the gas collector including an interface forming a gas collection area and including at least one nasal gas collecting inlet and an oral gas collecting inlet in fluid communication with the gas collection area, and a mouth engagement portion configured to project into the patient's mouth so as to maintain an open passage between the patient's mouth and the oral gas collecting inlet.

In one or more embodiments, the gas collector further includes an outlet for providing collected gases from the gas collection area to a gas analyser, a nasal gas collecting flow path in fluid communication between the nasal gas collecting inlet and the gas collection area; and an oral gas collecting flow path in fluid communication between the oral gas collecting inlet and the gas collection area.

In one or more embodiments, the gas collector is removably attachable to a non-sealing nasal interface.

Another aspect of the invention provides a gas collector for collecting gases at a patient, the gas collector including an interface configured to form a channel at an upper lip of the patient, one or more gas sampling conduits, each having a gas sampling inlet, for drawing gases from the channel to a localised gas collector area, and a gas flow diverter configured to direct at least some of the gases exhaled by a patient to one or more of the gas sampling inlets, wherein the gas flow diverter includes an upper portion that extends away from the patient's face.

In one or more embodiments, the gas collector further includes: a lower portion that extends towards the patient's face; and an intermediate portion interconnecting the upper and lower portions.

In one or more embodiments, the intermediate portion extends in a direction substantially parallel to the patient's face.

In one or more embodiments, the upper portion includes a top surface that, in use, sits underneath the patient's nose and is shaped to avoid creating a seal with the patient's nasal passages.

In one or more embodiments, the gas flow diverter has an inner surface which, in use faces the patient's face, and substantially follows the shape of the patient's face from nasal tip to upper lip.

In one or more embodiments, the inner surface has a substantially sigmoid shape.

In one or more embodiments, the spacer, in use, rests against the patient's upper lip; and wherein the gas collector further includes one or more nasal gas collecting conduits formed within the spacer, each nasal gas collecting conduit having a nasal gas collecting inlet in fluid communication with the patient's nasal passages, for delivering gases exhaled by a patient from their nasal passages to an outlet; an oral gas collecting conduit formed within the spacer, the oral gas collecting conduit having an oral gas collecting inlet in fluid communication with the patient's oral passage; and a gas flow diverter configured to direct at least some of the gases exhaled by a patient from their oral passage to the oral pas collecting inlet.

In one or more embodiments, the gas flow diverter is integrated with or attachable to the spacer.

Another aspect of the invention provides a gas collector for sampling gases at a patient, the gas collector including one or more nasal gas collecting conduits, each nasal gas collecting conduit having a nasal gas collecting inlet in fluid communication with the patient's nasal passages; and an oral gas collecting conduit, the oral gas collecting conduit having an oral gas collecting inlet in fluid communication with the patient's oral passage and the outlet, wherein the one or more nasal gas sampling conduits and the oral gas sampling conduit form a junction at localised gas collector area, and wherein the one or more nasal gas sampling conduits and the oral gas sampling conduit are configured so that a flow rate in the one or more nasal gas sampling conduits is a percentage of a total flow rate in the one or more nasal gas sampling conduits and the oral gas sampling conduit, where the percentage is within a predetermined range.

In one or more embodiments, the predetermined range is from about 1% to about 99%.

In one or more embodiments, the predetermined range is from about 5% to about 95%.

In one or more embodiments, the predetermined range is from about 20% to about 80%.

In one or more embodiments, the predetermined range is from about 45% to about 55%.

In one or more embodiments, the flow rate in the one or more nasal gas sampling conduits and is greater than the flow rate in the oral gas sampling conduit.

In one or more embodiments, the one or more nasal gas sampling conduits comprises a different resistance to flow to that of the oral gas sampling conduit to achieve the percentage in the predetermined range.

In one or more embodiments, the resistance-to-flow in the gas sampling conduits is achieved by modifying one or more of a length, cross-sectional area, a cross-sectional shape or an angular flow path of gas sampling conduits.

In one or more embodiments, the gas collector further includes a spacer configured to form a plurality of channels at an upper lip of the patient, the nasal and oral gas collecting conduits being formed within the spacer.

In one or more embodiments, the gas flow collector further includes a gas flow diverter configured to direct at least some of the gases exhaled by a patient from their oral passage to the oral gas collecting inlet.

In one or more embodiments, the gas flow collector further includes a connector interconnecting the oral gas sampling conduit and the localised gas collector area, the luer connector including a structure creating a tortuous fluid path increasing the resistance-to-flow of the oral gas sampling conduit.

In one or more embodiments, the gas collector is attachable to or integrally formed with a nasal cannula including nasal gas delivery prongs for delivering gases to the patient's nasal passages, wherein one of more of nasal gas collecting inlets are located proximate the patient's nasal passages, and wherein the nasal gas collecting inlets are arranged in one or more of the following positions: a first position wherein the nasal gas collecting inlets are substantially parallel to a length of the channel and facing away from a gas flow from the patient's nasal region; a second position, wherein the nasal gas collecting inlets are substantially traverse to a length of the channel; and a third position that between the first and second positions.

In one or more embodiments, there are two nasal gas collecting inlets and two gas sampling conduits in fluid communication with the two nasal gas sampling inlets, and one oral gas collecting inlets and one gas collecting conduit in fluid communication with the one oral gas sampling inlet, the gas collecting conduits from the two nasal gas collecting inlets and the one oral gas collecting inlet form a junction at localised gas collector area.

In one or more embodiments, the gas collecting conduits are formed within a spacer which, in use, rests against the patient's upper lip.

In one or more embodiments, the gas collector further includes an oral gas flow diverter configured to funnel at least some of the gases exhaled by a patient from their oral passage to the at least one oral gas sampling inlets.

Another aspect of the invention provides a patient interface including: a nasal cannula for delivering breathable gas to a patient; and a gas collector according to any one of the preceding claims, the gas collector being attached to or integrated with the nasal cannula.

One or more embodiments of the invention provide a gas collector having a structure which provides or creates one or more channels at an upper lip of the patient, for example in the patient's philtrum region, the one or more channels being in fluid communication with the nose and the mouth. Such a structure aids in channelling the exhaled gases from the nose and/or mouth to a localised area to increase gas sample concentration. Such a structure being in fluid communication with the nose and the mouth also aids in gathering a gas sample regardless of the patient breathing via the nose or the mouth. The gathered gas sample can then be provided to a gas analyser.

In one or more embodiments, the gas collector may be attachable to or integral with patient interfaces such as a nasal cannula.

### BRIEF DESCRIPTION OF DRAWINGS

Specific embodiments and modifications thereof will become apparent to those skilled in the art from the detailed description herein by reference to the figures follow, of which:

FIG. 1 shows a respiratory support system;

FIG. 2 shows a patient wearing a respiratory support system;

FIG. 3 shows a patient wearing a first embodiment of patient interface and a face mask;

FIG. 4 shows a cross-section of a portion of the patient interface of FIG. 3;

FIGS. 5 to 8 show a nasal cannula including a collapsible portion to which a gas collector can be attached or integrated with;

FIGS. 9 to 11 show another embodiment of patient interface;

FIG. 12 shows features or regions of a patient's face;

FIGS. 13 to 15 show three different types of channel structures;

FIGS. 16 to 20 show a first embodiment a gas collector;

FIGS. 21 to 23 show a nasal cannula with a gas collector of the type shown in FIGS. 16 to 20;

FIGS. 24 to 26 illustrates the gas collector of FIGS. 21 to 23 without the nasal cannula;

FIGS. 27 to 35 show a second embodiment of a gas collector;

FIGS. 36 to 38 show a third embodiment of a gas collector;

FIGS. 39 and 40 show a fourth embodiment of a gas collector;

FIGS. 41 to 44 show a fifth embodiment of a gas collector;

FIGS. 45 and 46 show a sixth embodiment of a gas collector;

FIG. 47 shows a seventh embodiment of a gas collector;

FIGS. 48 to 55 show different mechanism for attaching a gas collector to a patient interface;

FIGS. 56 to 59 show the gas collector of FIGS. 12 to 26 attached to a nasal cannula;

FIGS. 60 to 62 show a gas collector including integrated structural members;

FIGS. 63 to 66 show a variation to the gas collector of FIGS. 16 to 20 including a hinge;

FIGS. 67 to 70 show another variation to the gas collector of FIGS. 16 to 20 including a bi-stable structure;

FIGS. 71 to 76 show another variation to the gas collector of FIGS. 16 to 20 including openings or detachable portions for inserting and retaining medical equipment;

FIGS. 77 to 80 show another variation to the gas collector of FIGS. 16 to 20 including saliva-trapping open channels;

FIGS. 81 to 83 show another variation to the gas collector of FIGS. 16 to 20 including a modification central mouth engagement portion;

FIGS. 84 and 85 show another variation to the gas collector of FIGS. 16 to 20 including force absorbing sections;

FIGS. 86 to 89 show another variation to the gas collector of FIGS. 16 to 20 including a variation to the force absorbing sections of FIGS. 74 and 75;

FIGS. 90 to 97 show another variation to the gas collector of FIGS. 16 to 20 including an enlarged or enlargeable mouth engagement portion;

FIGS. 98 to 102 show another variation to the gas collector of 16 to 20 including means for attachment to a nasal cannula;

FIGS. 103 and 104 show another variation to the gas collector of FIGS. 16 to 20 including a recess at the base of curved nasal prongs to create a channel with the patient's upper lip;

FIGS. 105 to 107 show another variation to the gas collector of FIGS. 93 and 94 including a tube in fluid communication with a channel;

FIGS. 108 to 123 show another variation to the gas collector of FIGS. 16 to 20 including a sampling line to an outlet port and a series of a single action mechanisms to connect the sampling line;

FIGS. 124 to 127 show an eighth embodiment of a gas collector;

FIGS. 128 to 130 show a variation to the gas collector in FIGS. 124 to 127;

FIGS. 131 and 132 show a further variation to the gas collector in FIGS. 124 to 127; and

FIG. 133 shows a variation to the gas collector in FIGS. 131 to 132.

FIG. 134 shows a variation to the gas collector shown in FIGS. 16 to 20.

FIG. 135 is a schematic illustrating the gas collector of FIG. 134 as worn by a patient.

FIG. 136 is a further schematic showing the gas collector of FIG. 134 deforming when an instrument such as a laryngoscope is inserted in the patient's mouth.

### DETAILED DESCRIPTION

Various embodiments are described with reference to the figures. Throughout the figures and specification, the same reference numerals may be used to designate the same or similar components, and redundant descriptions thereof may be omitted.

In this specification, “high flow”, “high flows”, “high-flow” or other equivalent terminology means, without limitation, any gas flow with a flow rate that is higher than usual/normal, such as higher than the normal inspiration flow rate of a healthy patient. Alternatively, or additionally, it can be higher than some other threshold flow rate that is relevant to the context—for example, where providing a gas flow to a patient at a flow rate to meet or exceed inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult, child) and the like. Those skilled in the art know from context what constitutes “high flow”. It is a magnitude of flow rate that is over and above a flow rate that might otherwise be provided.

But, without limitation, some indicative values of high flow can be as follows.

In some configurations, delivery of gases to a patient at a flow rate of greater than or equal to about 5 or 10 litres per minute (5 or 10 LPM or L/min).

In some configurations, delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface via a system or from a flow source or flow modulator, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas delivered will be chosen depending on for example the intended use of a therapy and/or respiratory support. Gases delivered may comprise a percentage of oxygen. In some configurations, the percentage of oxygen in the gases delivered may be about 15% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, gases delivered may comprise a percentage of carbon dioxide. In some configurations, the percentage of carbon dioxide in the gases delivered may be more than 0%, about 0.3% to about 100%, about 1% to about 100%, about 5% to about 100%, about 10% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “high flow” for premature/infants/paediatrics (with body mass in the range of about 1 to about 30 kg) can be different. The flow rate can be set to 0.4-8 L/min/kg with a minimum of about 0.5 L/min and a maximum of about 70 L/min. For patients under 2 kg maximum flow may be set to 8 L/min. Also for example, for a 2 kg patient the flow rates would be about 0.8LPM to 16LPM.

High flow has been found effective in meeting or exceeding the patient's normal real inspiratory flow, to increase oxygenation of the patient and/or reduce the work of breathing. Additionally, high flow therapy and/or respiratory support may generate a flushing effect in the nasopharynx such that the anatomical dead space of the upper airways is flushed by the high incoming gas flows. This creates a reservoir of fresh gas available of each and every breath, while minimising re-breathing of carbon dioxide, nitrogen, etc.

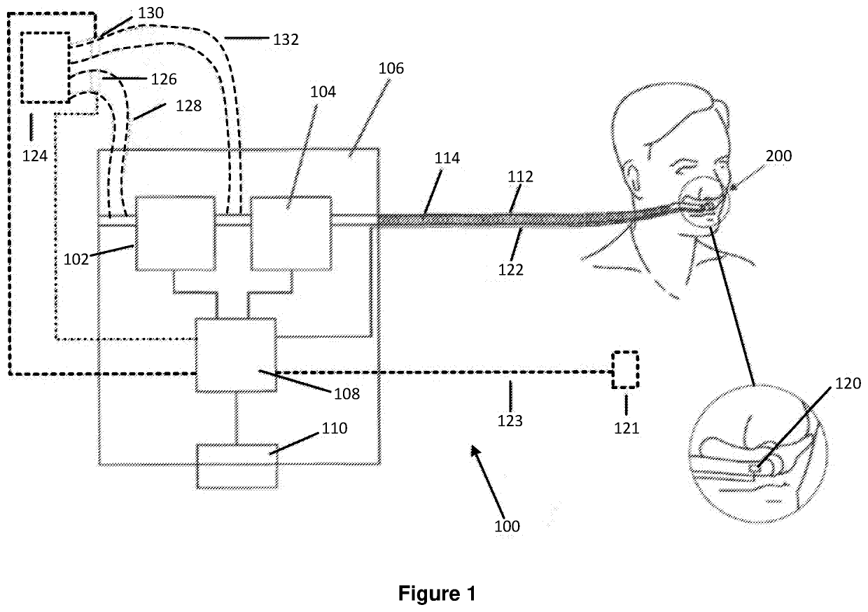

By example, a high flow respiratory system 100 is described below with reference to FIG. 1. High flow may be used as a means to promote gas exchange and/or respiratory support through the delivery of oxygen and/or other gases, and through the removal of CO2 from the patient's airways. High flow may be particularly useful prior to, during or after a medical and/or anaesthetic procedure.

When used prior to a medical procedure, high gas flow can pre-load the patient with oxygen (i.e. increase the reservoir of oxygen in the blood) so that their blood oxygen saturation level and volume of oxygen in the lungs is higher than normal in order to provide an oxygen buffer, which would be useful in reducing the risk or preventing desaturation for example when the patient is in an apnoeic phase during the medical procedure.

A continuous supply of oxygen is important to sustain healthy respiratory function during medical procedures (such as during anaesthesia) where respiratory function might be compromised (e.g. diminishes or stops). When this supply is compromised, conditions such as hypoxia and/or hypercapnia can occur. During medical procedures such as anaesthesia and/or sedation, patient breathing is monitored to detect if spontaneous breathing is diminished or ceases. If oxygen supply and/or CO2 removal is compromised, the clinician stops the medical procedure and facilitates oxygen supply and/or CO2 removal. This can be achieved for example by manually ventilating the patient for example through bag mask ventilation, or by providing a high flow of gases to the patient's airway using a high flow respiratory system or by jet ventilation. Further, it will be appreciated that a mask that is used for sedation/ventilation (not necessarily limited to a bag mask) may also be used for pre-oxygenation and also for monitoring patient parameters such as end tidal CO2, etc.

Further advantages of high gas flow can include that the high gas flow increases pressure in the airways of the patient, thereby providing pressure support that opens airways, the trachea, lungs/alveolar and bronchioles. The opening of these structures enhances oxygenation, and to some extent assists in removal of CO2 and/or can help support patients with collapsed areas of the lung.

When humidified, the high gas flow can also prevent airways from drying out, mitigating mucociliary damage, reducing risk of infection and reducing risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.

FIG. 1 shows a respiratory support system 100. The system 100 may be configured to provide high flow respiratory support and/or high flow therapy. The respiratory support system 100 comprises a flow generator 102. The flow generator 102 is configured to generate gas flows that are passed through the respiratory support system 100. The flow generator 102 is configured to generate gas flows that are provided to a patient at flow rates described elsewhere in the present specification. The flow generator 102 passes the air to a humidifier 104. The humidifier 104 is configured to heat and humidify gas flows (to temperatures and/or humidities as described elsewhere in the present specification) generated by the flow generator 102. In some configurations, the flow generator 102 comprises a blower adapted to receive gases from the environment outside of the respiratory support system 100 and propel them through the respiratory therapy system 100. In some configurations, the flow generator 102 may comprise some other gas generation means. For example, in some configurations, the flow generator 102 may comprise a source available from a hospital gas outlet (e.g. oxygen or air), or one or more containers of compressed air and/or another gas and one or more valve arrangements adapted to control the rate at which gases leave the one or more containers. As another example, in some configurations, the flow generator 102 may comprise an oxygen concentrator. In some configurations, the flow generator 102 may be adapted to deliver a high flow respiratory support and/or high flow therapy. In some embodiments, the flow source may include a compressed gas source, a device that modifies the flow from a compressed gas source and/or a flow generator which generates a gas flow.

The respiratory support system 100 comprises a housing 106 that at least partially houses both the flow generator 102 and the humidifier 104 (e.g. the respiratory support system 100 may comprise an integrated flow generator/humidifier apparatus). In other configurations the flow generator 102 and humidifier 104 may have separate housings, and/or be separate components. A hardware controller 108 is shown to be in electronic communication with the flow generator 102 and the humidifier 104, although in some configurations the hardware controller 108 might only communicate with the flow generator 102 or the humidifier 104. In some configurations, the flow generator 102 and the humidifier 104 may each have their own controller, which may or may not be in communication with one another. The hardware controller 108 may comprise a microcontroller or some other architecture configured to direct the operation of controllable components of the respiratory support system 100, including but not limited to the flow generator 102 and/or the humidifier 104.

An input/output module 110 is shown to be in electronic communication with the controller 108. The input/output module 110 may be configured to allow a user to interface with the controller 108 to facilitate the control of controllable components of the respiratory support system 100, including but not limited to the flow generator 102 and/or the humidifier 104, and/or view data regarding the operation of the respiratory support system 100 and/or its components. The input/output module 110 might comprise, for example, one or more buttons, knobs, dials, switches, levers, touch screens, speakers, displays and/or other input or output peripherals that a user might use to view data and/or input commands to control components of the respiratory support system 100.

As further shown in FIG. 1, a supplementary gas source 124 may be used to add one or more supplementary gases to the gases flowing through the respiratory support system 100. The one or more supplementary gases join the gas flow generated by the flow generator 102. The supplementary gas source 124 may be configured to deliver one or more supplementary gases including but not limited to air, oxygen (O2), carbon dioxide (CO2), nitrogen (N2), nitrous oxide (NO), anaesthetic agents and/or heliox (a mixture of helium and oxygen). The supplementary gas source 124 may deliver the one or more supplementary gases via a first supplementary gas conduit 128 to or towards the flow generator 102, and/or may deliver the one or more supplementary gases via a second supplementary gas conduit 132 to a location in the flow passage between the flow generator 102 and the humidifier 104. One or more supplementary flow valves 126, 130 may be used to control the rates at which the one or more supplementary gases can flow from the supplementary gas source 124 and through the first and/or second supplementary gas conduits 128, 132. One or more of the supplementary flow valves 126, 130 may be in electronic communication with the controller 108 or a separate controller, which may in turn control the operation and/or state of the one or more supplementary flow valves 126, 130. In other configurations, the supplementary gas source 124 may be configured to add one or more supplementary gases downstream of the humidifier 104. In other configurations, supplementary gas source 124 may be configured to add one or more supplementary gases into the humidifier 104, e.g. into a humidification chamber containing a body of water and engageable with a heater base, the water to be heated by a heating element to humidify a flow of gases to the patient.

As shown in FIG. 1, a conduit 112 extending from the humidifier 104 links the humidifier 104 to a patient interface 200. The conduit 112 may comprise a conduit heater 114 adapted to heat gases passing through the conduit 112. In other configurations the conduit heater 114 may not be present. In some embodiments, an optional filter (not shown) is arranged between conduit 112 and patient interface 200. The patient interface 200 is shown to be a nasal cannula, although it should be understood that in some configurations, other patient interfaces may be suitable. For example, in some configurations, the patient interface 200 may comprise a sealing or non-sealing interface, and may comprise a nasal mask, an oral mask, an oro-nasal mask, a full face mask, a nasal pillows mask, a nasal cannula, an endotracheal tube, tracheostomy tube, a combination of the above or some other gas conveying system. In an embodiment, the patient interface 200 is a non-sealing interface such as a nasal cannula, which allows gases to be exchanged with the environment. For example, the non-sealing cannula allows carbon dioxide to be removed and/or cleared from the patient's airways while the patient receives a gas flow from the system 100. Further, in some embodiments, the patient interface 200 is in the form of a nasal interface, such that the system does not interfere with other oral airway equipment and/or devices, for example, a tracheal tube in an intubation procedure.

Accordingly, the patient may continue to receive gas flow throughout the intubation procedure. In other embodiments, the patient interface 200 is an oral interface, for example an oral interface that is received in a user's mouth. An oral interface may be preferred in situations involving medical procedures via the nose, such that the interface does not interfere with nasal airway equipment and/or devices, for example a tracheal tube used in a nasal intubation procedure. In other embodiments the interface may be suitable for both nasal and oral placement or may be adapted between a nasal and an oral configuration.

As shown, in some configurations the patient interface 200 may also comprise a gas sensing module 120 adapted to measure a characteristic of gases passing through the patient interface 200. In some configurations, the gas collector (500; 700; 800; 850; 1000; 1040; 1060; 1080; 1090; 1110; 1116; 1122; 1138; 1140; 1150; 1170; 1180; 1190; 1200; 1220; 1254; 1362; 1274; 1282; 1300) of the present disclosure forms a part of the sensing module 120. The gas sensing module 120 could be located elsewhere within the gas delivery system and, for example, at the breathing conduit or humidifier. In some embodiments, there may be one or more gas sensing modules 120. In other configurations the gas sensing module 120 could be positioned and adapted to measure the characteristics of gases at or near other parts of the respiratory support system 100. The gas sensing module 120 may comprise one or more sensors adapted to detect the presence of gases and/or measure various characteristics of gases, including but not limited to pressure, flow rate, temperature, absolute humidity, relative humidity, enthalpy, gas composition, oxygen concentration, carbon dioxide concentration (e.g. for determining end tidal CO2), and/or nitrogen concentration. Gas properties determined by the gas sensing module 120 may be utilized in a number of ways, including but not limited to closed loop control of parameters of the gases. For example, in some configurations flow rate data taken by a gas sensing module 120 may be used to determine the instantaneous flow, which in turn may be used to determine the respiratory cycle of the patient to facilitate the delivery of flow in synchronicity with portions of the respiratory cycle. The gas sensing module 120 may communicate with the controller 108 over a first transmission line 122. In some configurations, the first transmission line 122 may comprise a data communication connection adapted to transmit a data signal. The data communication connection could comprise a wired data communication connection such as but not limited to a data cable, or a wireless data communication connection such as but not limited to Wi-Fi or Bluetooth. In some configurations, both power and data may be communicated over the same first transmission line 122. For example, the gas sensing module 120 may comprise a modulator that may allow a data signal to be ‘overlaid’ on top of a power signal. The data signal may be superimposed over the power signal and the combined signal may be demodulated before use by the controller 108. In other configurations the first transmission line 122 may comprise a pneumatic communication connection adapted to transmit a gas flow for analysis at a portion of the respiratory support system 100. In other configurations, the transmission line 122 comprises a pneumatic communication connection separate from the conduit 112, adapted to transmit a captured gas flow at the patient for analysis at a separate system or device such as a capnograph.

Additionally as shown a physiological sensor module 121 may be present. The physiological sensor module 121 may be configured to detect various characteristics of the patient or of the health of the patient, including but not limited to heart rate, EEG signal, EKG/ECG signal, inertial sensors attached to the patient (e.g. to the chest) to detect movement, blood oxygen concentration (via, for example, a pulse oximeter), blood CO2 concentration, transcutaneous CO2 (TcC02) and/or blood glucose. Similarly, the physiological sensor module 121 may communicate with the controller 108 over a second transmission line 123. The second transmission line 123 may comprise wired or wireless data communication connections similarly to the first transmission line 122, and power and data may be communicated similarly. The physiological sensor module 121 may be used, for example, to determine the blood oxygen saturation of the patient. In some embodiments, the second transmission line 123 may comprise a pneumatic communication connection adapted to transmit a fluid for analysis at a portion of the respiratory support system 100 or at separate system or device.

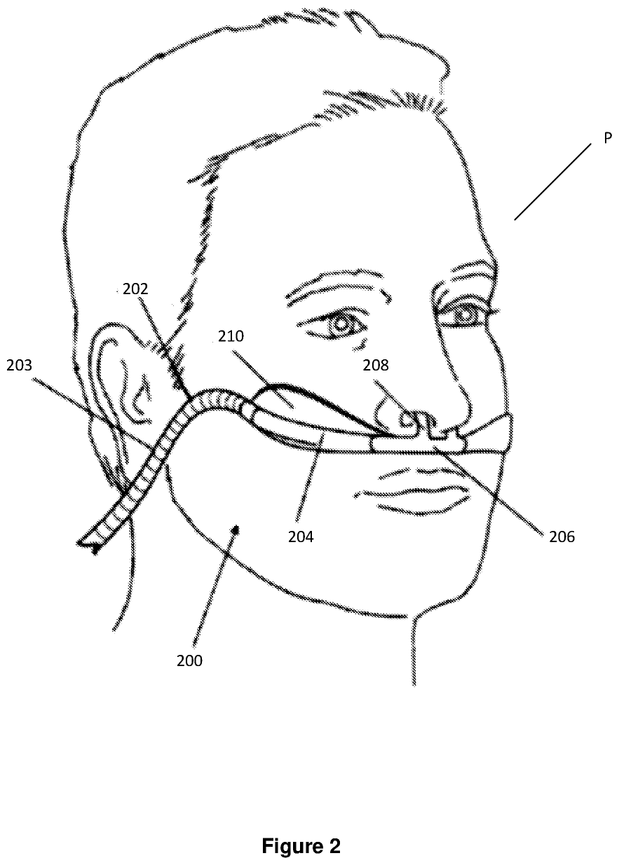

FIG. 2 shows a user or patient P wearing a patient interface 200, for example the patient interface 200 of the respiratory system of FIG. 1. The patient depicted is an adult, however, the patient may be an infant, a neonate or a child. In the illustrated non-limiting configuration, the patient interface 200 is a nasal cannula. The patient interface 200 comprises a first gas conduit 202. The first gas conduit 202 is adapted to receive gases from the respiratory support system 100 (for example, via the conduit 112 shown in FIG. 1) and channel the gases to the patient P. The first gas conduit 202 may comprise a reinforcement element 203 adapted to strengthen and/or add rigidity to the first gas conduit to prevent deformation or collapse of the first gas conduit 202 arising due to the application of forces against the first gas conduit 202. The reinforcement element 203 may include a number of structures, including but not limited to plastic or metallic reinforcing beads that lie in or on the wall of the first conduit lumen 202.

The first gas conduit 202 is in pneumatic communication with a flow manifold 206. The flow manifold 206 receives gases from the first gas conduit 202 and passes them to one or more nasal delivery elements 208 (e.g. nasal prongs). The one or more nasal delivery elements 208 extend outwardly from the flow manifold 206. The one or more nasal delivery elements 208 are adapted to be non-sealing (i.e. a gap exists between each nasal delivery element and the patient's nasal passage) when positioned in one or more nares of the patient P. As shown, the patient interface 200 comprises two nasal prongs 208 adapted to be positioned one in each of the patient's nares. Each nasal prong 208 may be shaped or angled such that it extends inwardly towards a septum of the patient's nose. Alternatively, the first patient interface 200 may be a sealing nasal interface.

In the embodiment shown in FIG. 2, the flow manifold 206 receives flow from one lateral side of the flow manifold 206 (e.g. with respect to an imaginary vertical plane bisecting the face of the patient P) and channels flow to the manifold and each of the nasal prongs 208. In some configurations, the flow manifold 206 receives flow from a single side of the flow manifold 206 and channels flow to the manifold and each of the nasal prongs 208. The single side may be a single lateral side. In some embodiments a conduit may extend from the left hand side or from the right hand side of the manifold. In some situations providing the conduit on the left hand side of the patient interface may be preferred for access for a clinician, for example for intubation. Alternatively, a conduit extending from the right hand side may be preferred, for example in procedures such as endoscopies where the patient is typically lying on his or her left hand side. In other configurations, the patient interface 200 may comprise greater (for example, three or four) or fewer (for example, one) nasal delivery elements 208. In other configurations, each nasal delivery elements 208 can have different structures, dimensions, shapes and/or properties. For example, one of a pair of nasal delivery elements 208 can be relatively long and the other nasal delivery elements 208 can be relatively short.

In some configurations, the flow manifold 206 may be configured to receive flow from two lateral sides of the flow manifold 206 (e.g. from a ‘left’ and ‘right’ of the flow manifold 206 instead of just the patient's right hand side of the flow manifold 206 as seen in FIG. 2). In some such configurations, multiple gas conduits may be used to provide for pneumatic communication between the flow manifold 206 and the respiratory support system 100. For example, the patient interface may comprise dual conduits, the first gas conduit 202 extending from a first side of the interface (in the illustrated example the right hand side of the patient) and a second gas conduit extending from a second opposite side of the interface. In some configurations, the flow manifold 206 may be configured to receive flow from a non-lateral side of the flow manifold 206 (e.g. from a ‘bottom’ or ‘top’ of the flow manifold 206). In some configurations, the flow manifold 206 may receive flow more than one gas conduit from a single side, optionally a single lateral side of the manifold 206. In some configurations, one prong may receive flow from one gas conduit and the other prong may receive flow from another separate gas conduit.

The patient interface may further comprise mounts and/or supports, e.g., cheek supports 210, for attaching and/or supporting the gas conduit 202 or conduits on the patient's face. Alternatively or additionally, the patient interface may be held in place via one or more headstraps or headgear.

The first gas conduit 202 may comprise a first portion 204 configured to transition from a first configuration in which a first level of gases is able to pass through the first portion 204 to a second configuration in which a second level of gases is able to pass through the first portion 204.

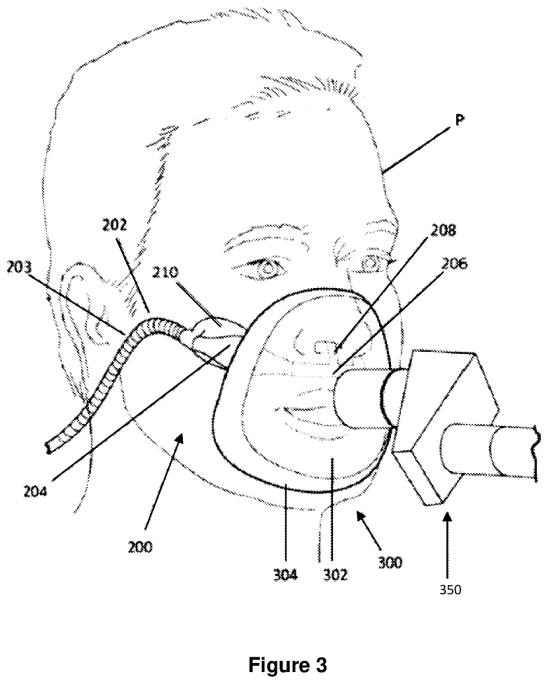

FIG. 3 shows a non-limiting exemplary embodiment of a patient P wearing the patient interface 200 as shown in FIG. 2 (a first patient interface) underneath a face mask 300 assembly (a second patient interface). FIG. 3 schematically shows the face mask as a transparent structure in order to illustrate the patient interface 200 under it. The first patient interface 200 may be used with a first respiratory support subsystem and the second patient interface 300 may be used together with a second respiratory support subsystem. In some embodiments, the first patient interface 200 and second patient interface 300 may be used with the same respiratory support system.

A system may find benefit in the selective delivery of separate respiratory supports and/or therapies to a patient using different patient interfaces, and/or in stopping or ceasing the delivery of a respiratory support and/or therapy from an interface and/or allowing gases provided by an interface to be sampled.

The system and devices as described find particular application in emergency resuscitation, around intubation of a patient receiving high flow respiratory support and/or therapy, ear, nose, and throat (ENT) surgery, in assisting with conditioning of a patient in a pre-operative state prior to administration of anaesthetics, and during post-extubation and recovery.

Face mask assembly 300 may be used as or with a second respiratory support subsystem and/or to deliver one or more substances other than a substance delivered by the cannula 200, for example anaesthetic agents or oxygen, to the patient, or the same substance but at different flow and/or pressure levels. Alternatively, the face mask assembly 300 may be used to stop the delivery of respiratory support and/or therapy from a first respiratory support subsystem. The face mask assembly 300 may also be adapted to measure respiratory gases, for example exhaled carbon dioxide from the patient, the measurements of which may otherwise be affected by flow from the patient interface 200 of the first respiratory support subsystem.

Accordingly, the embodiment shown in FIG. 3 allows for the alternation between the two different respiratory support subsystems. Additionally, this configuration may allow the patient interface 200 to be left on the patient throughout the surgical procedure and/or into recovery (whether or not the patient continues to receive a gas flow through the patient interface 200 throughout the procedure) without interfering with other clinical practices.

In the embodiment shown, face mask assembly 300 comprises a full face mask 302 configured to cover both the patient's nose and mouth. In other configurations, the face mask 300 may be a nasal mask which is placed over the patient interface 200 to cover only the patient's nasal region. In such configurations, a portion of the face mask 300 may be placed upon a portion of the patient interface 200, such as first portion 204.

As shown, the face mask 302 comprises a seal region 304 adapted to seal against the patient's face. The face mask assembly 300 is connected to a second gas source, for example via a filter element 350 or a humidity moisture exchanger (not shown), which supplies the one or more other gases to the patient via the face mask. That is, the second gas source is preferably different from the source supplying gas (for example, supplementary gas source 124/flow generator 102) to the patient interface 200. In other embodiments, the patient interface 200 and the face mask assembly 300 are connected to a common gas source.

In an embodiment, the face mask assembly 300 is connected to a separate gas source or a separate respiratory support device. For example, the respiratory support can be a ventilator or a CPAP or a high flow respiratory support and/or therapy device or a manual resuscitator (for example a hand-held face mask with bag). Alternatively or in addition, the face mask assembly 300 may be connected to a device for measuring a characteristic of respiratory gases.

Alternatively, the mask assembly 300 could be connected to an anaesthetic device and anaesthetic gas, or air, or oxygen, or a combination of gases, can be delivered via the mask 302.

The embodiment shown in FIG. 3 allows for the delivery of gas from multiple sources via at least two different respiratory support modes, and further allows a doctor, clinician or medical professional to quickly and easily change the type of respiratory support mode.

In one particular application, a patient preparing for anaesthesia can be pre-oxygenated by delivering a high flow of oxygen or humidified gases or mixture of both via a nasal cannula. In some circumstances, anaesthesiologists managing the sedation and/or anaesthesia of a patient may want to switch between delivery of gas flow from one patient interface (for example a nasal cannula 200) and delivery of gas flow from another patient interface, such as via a face mask 300.

Anaesthesiologists also use a mask with a bag to oxygenate a patient, and in some instances find it more beneficial to use a bag mask if a patient's vital signs begin to drop for example to deliver more pressure or have greater control over the variation in delivered pressure. In some situations, a medical professional may wish to switch between different respiratory systems or support modes. In a first mode respiratory support may be provided by a first respiratory support system (for example via the patient interface 200) and in a second mode respiratory support may be provided by a second respiratory support system (for example via the patient interface 300), with the support from the first system reduced or stopped. For example, the additional flow from a high flow provided by nasal interface 200 may also modify the expected behaviour of the anaesthetic circuit provided by the face mask 300, and therefore it may be advantageous to be able to reduce or stop the additional flow from the first respiratory system.

In some configurations, the switching between two respiratory support modes or subsystems may be facilitated by a structure of the first gas conduit 202, which has first portion 204 configured to transition from a first configuration in which a first level of gases is able to pass through the first portion 204 to a second configuration in which a second level of gases is able to pass through the first portion 204.

In some configurations, the first portion 204 is configured to be more collapsible or otherwise better adapted at changing the flow of gas through the first portion 204 (therefore stopping or reducing the flow of gas through the conduit and to the patient) than other portions of the conduit 202, and/or allowing a seal of a mask to seal over the top of the conduit. In other configurations the entire conduit may be configured to be collapsible. In some configurations a vent arrangement may be provided to vent gases from the conduit to atmosphere.

In some embodiments, the first configuration or first condition is a substantially open configuration and the second configuration or second condition is a substantially closed configuration. That is, the conduit 202 is configured to be more collapsible, deformable or otherwise adapted to fully close off the flow at the first portion 204 than at other portions of the conduit 202. In the second condition, gases to the nasal delivery elements 208 may be reduced or stopped.

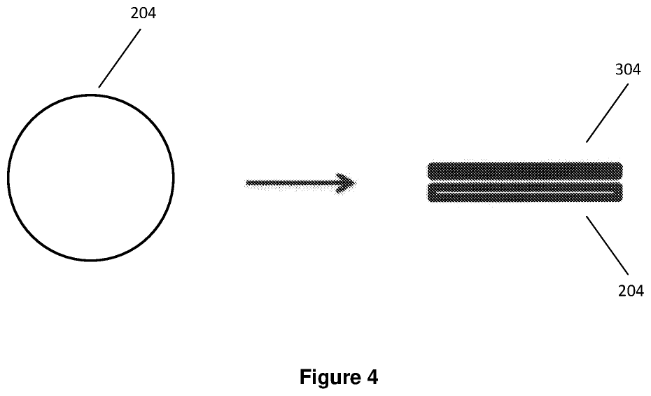

FIG. 4 shows one example of this configuration, in which the conduit (for example the conduit 204 of the nasal cannula 200 of FIG. 3) at a first portion 204 is substantially closed by the seal 304 of face mask 302. In such an embodiment, the first portion (i.e. the more collapsible or deformable section) of the first gas conduit should be of a length that is greater or equal to a width of a section of a seal of the face mask that bears over the first portion of the first gas conduit. This may provide that the seal of the face mask does not bear over a non-collapsible section of the first gas conduit. For example, the first portion may extend from a distance of 35 mm or less from a portion of the manifold 206 or the centre of a user's nose to at least 50 mm from a portion of the manifold 206 or the centre of a user's nose, The first portion 204 may have a length of at least about 5 mm, about 1 mm to about 30 mm in length, or about 5 mm to about 15 mm in length, or about 10 mm in length. In some embodiments the length of the first portion may be at least 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 20 mm, 25 mm, 30 mm, 35 mm, 40 mm, 45 mm, 50 mm or greater.

The first portion 204 may progress between the first and second configurations based on a relative level of force applied to a wall of the first portion 204. For example, as shown in FIG. 3, the force may be applied by the seal 304 of face mask 302. In this example, first portion 204 is configured to be positioned under the seal 304 of the face mask 302.

Alternatively, the force may be applied to first portion 204 by other means, e.g., clamps (not shown), or alternatively a medical practitioner may compress the conduit by pressing on the conduit wall with a finger or thumb.

In some embodiments, the seal of the face mask acting on the first portion 204 of the gas conduit causes the first portion 204 to form a seal or at least an occlusion between the nasal outlets of the first patient interface 200 and the flow generator 102. Additionally, the seal of the face mask forms a seal or at least a partial seal over the first portion 204 of the gas conduit 202.

Switching between respiratory support therapies is therefore achieved simply by applying a mask to the patient's face so that the seal of the mask collapses (partially or completely) the first portion of the gas conduit of the first interface 200 to stop or ‘turn off’ or reduce the respiratory support and/or therapy supplied by the first interface 200 and also provides a seal between the face mask 300 and the external surface of the first portion 204 of the conduit 202 such that respiratory support and/or therapy can be provided by the mask 300 with the respiratory support and/or therapy provided by the first interface 200 is stopped or reduced. As noted, the first portion 204 of the patient interface 200 is configured to be collapsible and will hereinafter be referred to as collapsible portion 204.

The cannula with a collapsible conduit portion allows a user, e.g. an anaesthetist or a nurse or a clinician to use a mask and prevent delivery of gases from multiple sources (e.g. the mask and cannula). The first interface 200 is structured and functions in a manner to reduce or close the delivery of high flow and allow delivery of other respiratory support and/or respiratory therapy or anaesthesia gases through a mask when the interface 200 is moved to a collapsed configuration. In some embodiments the removal of the mask from the patient's face allows the respiratory support and/or therapy supplied by the first interface to recommence, as the conduit returns from the collapsed configuration to the open configuration.

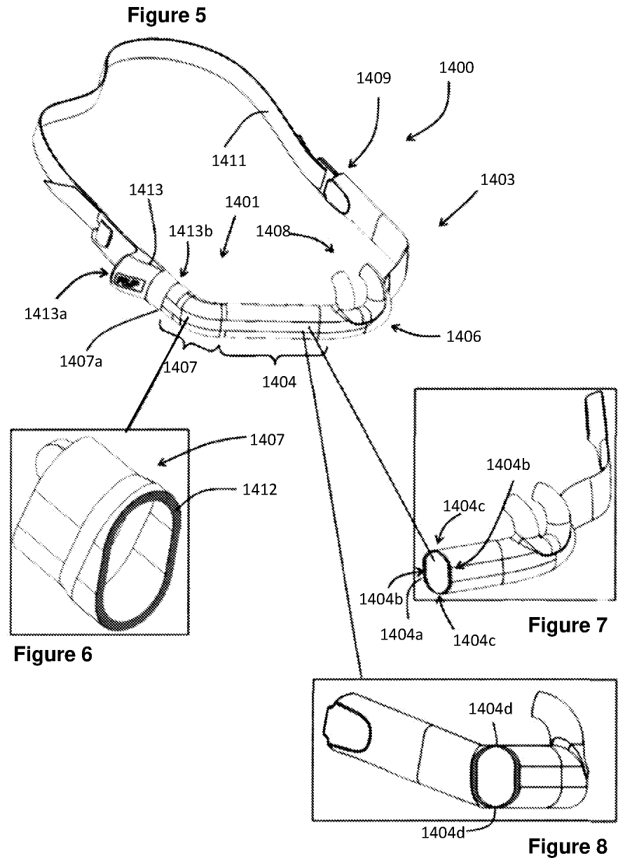

FIGS. 5 to 8 exemplify a patient interface 1400 comprising a nasal cannula and including a gases delivery side member 1401 configured to deliver apparatus gases (for example gas flow from a flow source) to a patient via a manifold 1406 to a delivery outlet comprising a pair of nasal prongs 1408. The pair of nasal prongs 1408 extend from the manifold 1406. The gases delivery side member 1401 extends from a first side of the manifold 1406 and the interface 1400 further includes a non-delivery side member 1403 extending from a second side of the manifold 406 which is opposite to the first side. The non-delivery side member 1403 includes an end 1409 configured for connection to a headstrap 1411.

The gases delivery side member 1401 includes a collapsible portion 1404 configured to move from the normally open configuration shown in FIGS. 5, 7 and 8 to a collapsed configuration in which apparatus gas flow through the collapsible portion 1404 is reduced or stopped. The collapsible portion 1404 is configured to move to the collapsed configuration upon application of a collapsing force such as from a patient mask placed over the patient's face and wherein a seal of the mask is pressed down upon the collapsed portion 1404. The gases delivery side member 1401 also includes a non-collapsible portion 1407 configured to remain open during application of the collapsing force onto the collapsible portion 1404.

One end of the non-collapsible portion 1407 comprises a delivery inlet 1407a for receiving apparatus gas flow. The patient interface 1400 further includes a gas path connector 1413 which has a rigid structure and includes a delivery inlet 1413a and a delivery outlet 1413b. The gas path connector delivery inlet 1413a is connectable to an apparatus gas supply via a conduit (not shown). The gas path connector delivery outlet 1413b is connected to the delivery inlet 1407a of the non-collapsible portion 1407. The gas path connector 1413 is also connected to the headstrap 1411 at an opposite end of the headstrap to that which is connected to the headstrap end 1409 of the non-delivery side member 1403.

FIG. 6 illustrates a cross-section of the non-collapsible portion 1407 which includes wall 1412 of uniform thickness. FIGS. 7 and 8 illustrate a cross-section of the collapsible portion 1404 which includes a wall 1404a of non-uniform thickness. The collapsible portion 1404 has an elongate cross-section and in particular a stadium-shaped cross section which includes a pair of longitudinal sides 1404b extending between a pair of ends 1404c. As shown in FIG. 8, a thin-walled portion 1404 is provided at each of the ends 1404c. The thin wall portions 404 d and 1404d are configured to provide fold lines at which the collapsible portion 1404 bends or folds upon application of the collapsing force.

The patient interface 400 illustrate in FIGS. 5 to 8 provides context for various embodiments of patient interfaces (or parts thereof) similar to interface 1400 described herein.

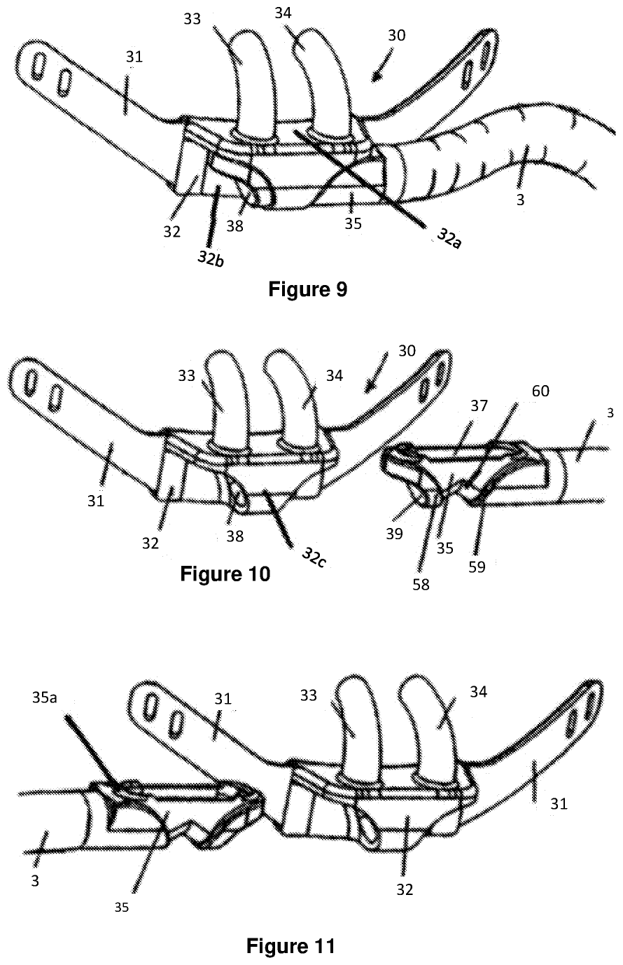

Another example of a patient interface is shown in FIGS. 9 to 11. The nasal cannula 30 comprises a face mount part 32, a pair of nasal prongs 33, 34, gases flow manifold part 35 and gas conduit 32. The face mount part 32 comprises an upper (first) portion 32a from which the prongs 33, 34 extend and a face contacting (second) portion 32b that is configured to contact the patient's upper lip in use. The upper portion 32a and face contacting portion 32b are at an angle to one another and are optionally substantially perpendicular to one another. The face mount part 32 and pair of nasal prongs 33, 34 are preferably integrally molded as one piece from a soft plastics material such as silicone or thermoplastic elastomer, although in other forms the face mount part and prongs may be separate, but capable of attachment together for use. In some configurations, the face mount part 32 and the nasal prongs 33, 34 may be formed from the same or different material. The nasal prongs 33, 34 may be tubular in shape and may be consistent in diameter but may be shaped to fit the contours of the human nares. The prongs 33, 34 may be angled towards a central plane bisecting the face mount part 32 between the prongs. The prongs 33, 34 may be curved to point the outlets of the prongs 33, 34 toward the back of the patient's head when in use. The prongs 33, 34 may comprise an internal and/or external cross-sectional shape transverse to a direction of flow through each prong when in use, that is elliptical, for example a circle or substantially elliptical, for example an oval. The shape and/or dimensions of each prong 33, 34 may be consistent or may change along its length. The prongs 33, 34 are configured to be non-sealing with the patient's nares in use such that there is a gap between the prongs and the patient's nares. This allows continuous flow of gas between the prongs and the patient's nares when in use.

The face mount part 32 comprises side arms 31 that extend laterally from the sides of the face mount part 32. Together with a headstrap (not shown), the side arms 31 help hold the nasal cannula 30 in place on a patient's face. The ends of each side arm 31 comprises one or more slots to allow an end of a headstrap to thread through. This may provide for an adjustable coupling between the headstrap and the side arm 31. Other attachment mechanisms other than slots are also envisaged, such as buckles and clips.

The face mount part 32 further comprises a third portion 32c extending from the upper portion 32a and connects to the face contacting portion 32b to form a recess 38 that is capable of receiving the gases flow manifold part 35. In the embodiment shown, the recess 38 provides for a horizontal side entry of the gases flow manifold 35. The gases flow manifold 35 may therefore be laterally inserted into the recess 38 via one lateral side of the face mount part 32, in a direction that is transverse to the length of the prongs 33, 34. As the recess 38 comprises two lateral openings, the gases manifold part 35 may be inserted into the recess 38 via a left or a right side of the face mount part 32. This allows the nasal cannula 30 to be configured to allow gases flow to the patient from either the left or right side of the nasal cannula. The gases flow manifold 35 may be attached to or integrally formed with gas conduit 3. The nasal prongs 33, 34 comprise flow passages that extend through the face mount part 32 and into the recess 38. The assembly of the face mount part 32 and the gases flow manifold 35 comprises a manifold. The gases flow manifold part 35 is blocked at one end 39 but attached to the gas conduit 35 at the other end. The gases flow manifold 35 has an opening 37 that acts as an exit for gases received from the gas conduit 3. The opening 37 is shown as an elongate opening but other shapes are also envisaged. The gases flow manifold 35 may be more rigid or comprise a material that is more rigid than the face mount part 32. Due to the relative rigidities/flexibilities of the gases manifold part 35 and face mount part 32, the gases flow manifold part 35 can be pushed through the recess 38 in the face mount part 32 and the opening 37 in the gases flow manifold part 35 meets with the flow passages of the prongs 33, 34. Therefore, in use, gases flowing through the gas conduit 3 and into the gases flow manifold part 35 exit through the opening 37 and into the tubular passageways in the prongs 33, 34, then into the patient's nares.

In order to assist with maintaining the gases flow manifold part 35 within the recess 38, the gases flow manifold part 35 is provided with a recessed portion 60 and lip areas 58, 59. When engaged with the face mount part 32, the third portion 32c forming part of the recess 38 sits within the recessed portion 60 and the edges of the third portion 32c about the lips 58, 59 formed on the gases flow manifold part 35. Additionally, or alternatively, the gases flow manifold 35 comprises one or more flanges 35a that is configured to engage with a part of the upper portion 32a to retain the gases flow manifold 35 with the face mount part 32. The one or more flanges 35a is located about a periphery of opening 37. In some configurations, the one or more flanges 35a is a single flange that extends about the entirety of the periphery of opening 37.

Embodiments of the gas collector described below are attachable to or integral with the nasal cannula 30, 200 and/or 1400.

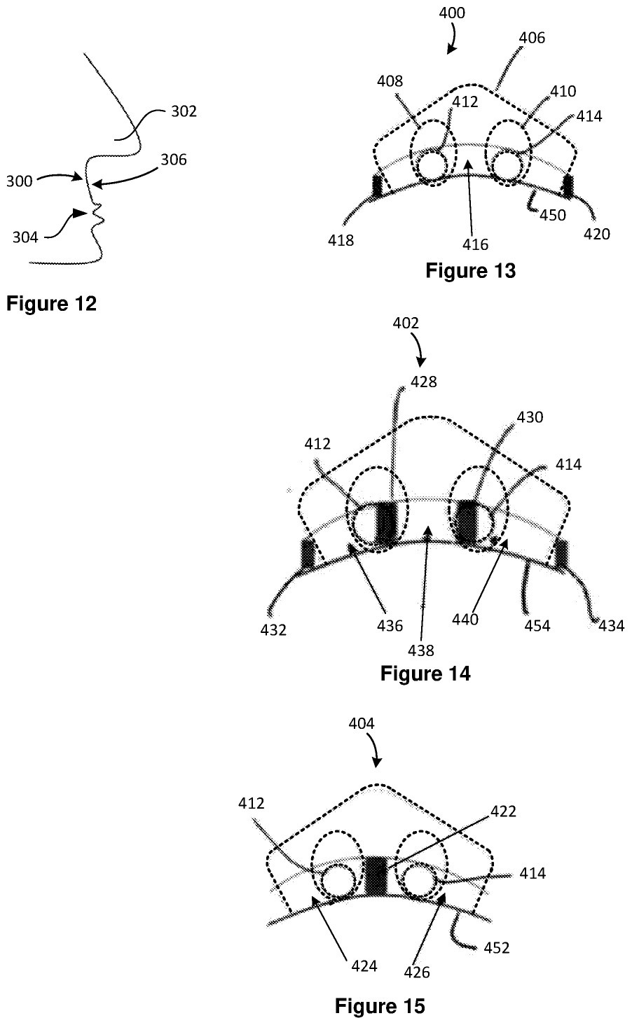

Various embodiments of the gas collector provide or create one or more channels at or around an upper lip 300 of a patient. In some configurations, the one or more channels may be formed partly by a portion of the gas collector and partly by the patient's upper lip when the gas collector is in use. The one or more channels being in fluid communication with the patient's nose 302 and mouth 304, as shown in FIG. 12. The one or more channels provide a passage for fluid communication between the patient's nose 302 and mouth 304 regions. In other words, the patient's nose region 302 is in fluid communication with the patient's mouth region 304 via the one or more channels. The one or more channels is open to atmosphere at least at the patient's nose and/or mouth regions. The one or more channels may, in certain embodiments, be provided more particularly at or around the patient's philtrum region 306. The one or more channels aids in gathering gases from the nose 302 and/or the mouth 304 regions to a localised area for collection for sampling and analysis. In some configurations, the one or more channels aid in gathering exhaled gases from the nose 302 and/or mouth 304 to a localised area to increase the concentration of exhaled gases prior to sampling and analysis. Having one or more channels that provide a passage for fluid communication between the nose 302 and the mouth 304 also assists in collecting a gas sample regardless of the patient breathing via the nose or the mouth. In use, a portion of the exhaled gases that exit the nose and mouth travel to the upper lip region, hence the one or more channels creates a catchment region around the upper lip which is beneficial in collecting gases from the nose and/or mouth regions, especially during provision of high flow gases.

In one or more embodiments, the one or more channels may be created in part by a portion of a nasal cannula's preform curvature. Additionally or alternatively, a device integral with or removably attachable to a nasal cannula may be used to form these channels.

FIGS. 13, 14 and 15 respectively depict three different types of channel structures, respectively referenced 400, 402 and 404. These images are depicted looking from the chin of a patient looking up to their head, and the outline of the patients nose 406 and nostrils 408 and 410, as well as nasal prongs 412 and 414 for delivering gas to a patient's nasal passages, are depicted by broken lines.

Spacers, depicted as solid blocks, maintain a portion of a channel wall away from the patient's upper lip 450, 452, 454. In some embodiments, the channel wall portion spaced from the patient's upper lip may be formed by a portion of a nasal cannula to which the gas collector is attached or integrally formed with, however in other embodiments the channel wall portion may be a device separately formed from the nasal cannula.

Whilst the channels depicted in FIGS. 13 to 15 are shown to be partly formed by the patient's upper lip, in other embodiments the channel may be created by a pre-formed body that includes one or more gas collecting conduits formed within that simply rests against the patient's upper lip.

The first channel type 400 shown in FIG. 13 includes a channel 416 that is bound by two spacers 418 and 420, the channel including a first open end facing the patient's nose and a second open end facing the patient's mouth. The spacers 418 and 420 are configured to abut against the lateral sides of the patient's upper lip in use, on either side of the philtrum region. With respect to a nasal cannula, the spacers 418 and 420 are positioned laterally with respect to the nasal prongs 412 and 414. In other words, from a top planar view, the prongs 412 and 414 are positioned between spacers 418 and 420. The spacers 418 and 420 are configured to be positioned inferior to the nasal prongs 412 and 414 insofar as the upper lip is positioned inferior to the nares. In some embodiments, the spacers 418 and 420 are positioned directly inferior to the nasal prongs 412 and 414. Gases exhaled by the patient or provided to the patient via a nasal cannula, and which enter the channel, can be collected from multiple locations within that channel. The collected gases can then be analysed in situ or delivered to a gas analyser for analysis. The gas analyser may comprise a negative flow source that draws the collected gases at a flow rate of about 40 ml/min to about 500 ml/min. In some embodiments, the spacers 418 and 420 may be integral with or attachable to a nasal cannula.

The second channel type 404 shown in FIG. 15 includes a spacer 422, located substantially along a central plane of the gas collector. When in use, the spacer 422 is configured to contact the philtrum region at the upper lip of the patient and be located inferior to and substantially between the patient's nares, as well as prongs 412 and 414 of a nasal cannula. Location of the spacer 422 in this manner creates two open channels 424 and 426 on either side of the spacer 422, both of which are in fluid communication with the nasal passages and the upper lip. The open channels 424 and 426 are also in fluid communication with the patient's oral passage and the upper lip (not shown). Channels 424 and 426 are formed to promote fluidic communication between these areas. Collected gases can be drawn from one or multiple locations within the channels thus formed.

In the third channel type 402 shown in FIG. 14, spacers 428 and 430, when in use are configured to contact the patient's upper lip, and may be disposed under the patient's nares, and optionally under nasal prongs 412 and 414. Further, additional spacers 432 and 434 may be located inferior to the patient's nares and be disposed on either side of the nasal prongs 412 and 414. These additional spacers 432 and 434 may contact a portion of the patient's nasolabial folds. This results in the creation of three separate channels 436, 438 and 440. Once again, these open channels are created between the nose, mouth and upper lip region of a patient to enable gases to be collected at a sampling area. A sample of the exhaled gases may for example be drawn from these sampling areas for further analysis.

In some embodiments, the spacers 418, 420, 422, 432, 434, 428 and 430 are attachable or formed integral with the gas collector. In some embodiments, the gas collector may be a separate device removably attachable or integral with a nasal interface such as a nasal cannula shown in 30 and/or 200.

It will be appreciated that the arrangements depicted in FIGS. 13 to 15 are exemplary only and are intended to show three non-exhaustive manners in which a channel wall portion can be spaced from a patient's face by means of spacers in order to create open channels enabling the collection of gases exhaled by a patient from the nasal passages and oral passage.

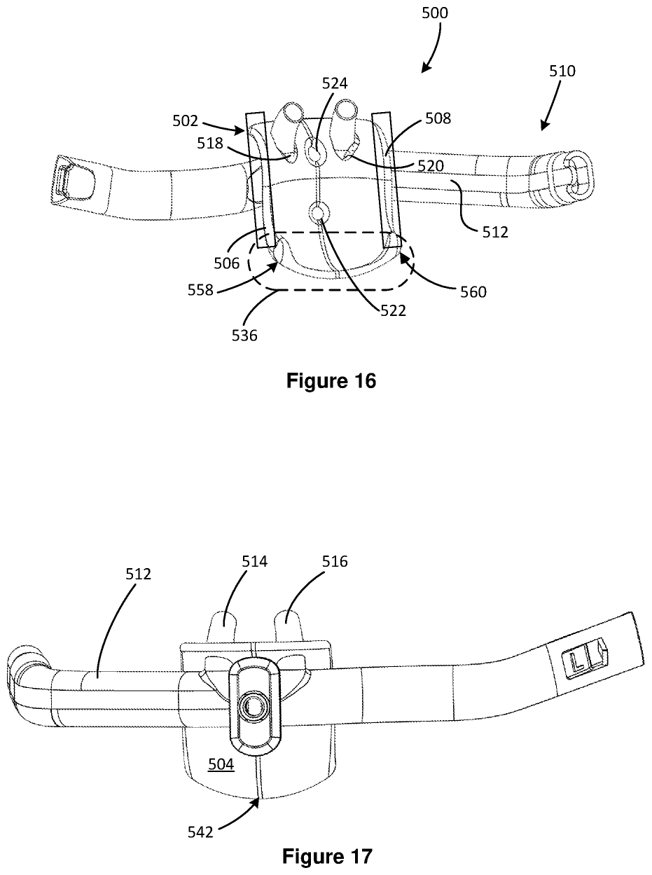

FIGS. 16 to 18 show a first embodiment of a gas collector 500 for collecting gases exhaled by a patient from their nasal passages and oral passage. The gas collector 500 includes an interface 502 configured to form a channel with the upper lip of the patient, the channel having open ends respectively in fluid communication with the patient's nasal passages and oral passage. In this example, the interface 502 has “scoop” portion including a curved channel wall portion 504 spaced from a patient's face by two spacers 506 and 508. In this example, the spacers 506 and 508 are in the form of ribs extending along the length of the curved channel wall portion 504 and projecting from that channel wall portion 504 towards the patient's face in order to provide the required spacing. The spacers 506 and 508 are common with this example, integral with the channel wall portion 504. The spacers 506 and 508 are common in this example, disposed to contact a patient's face on either side of the nasal passages.

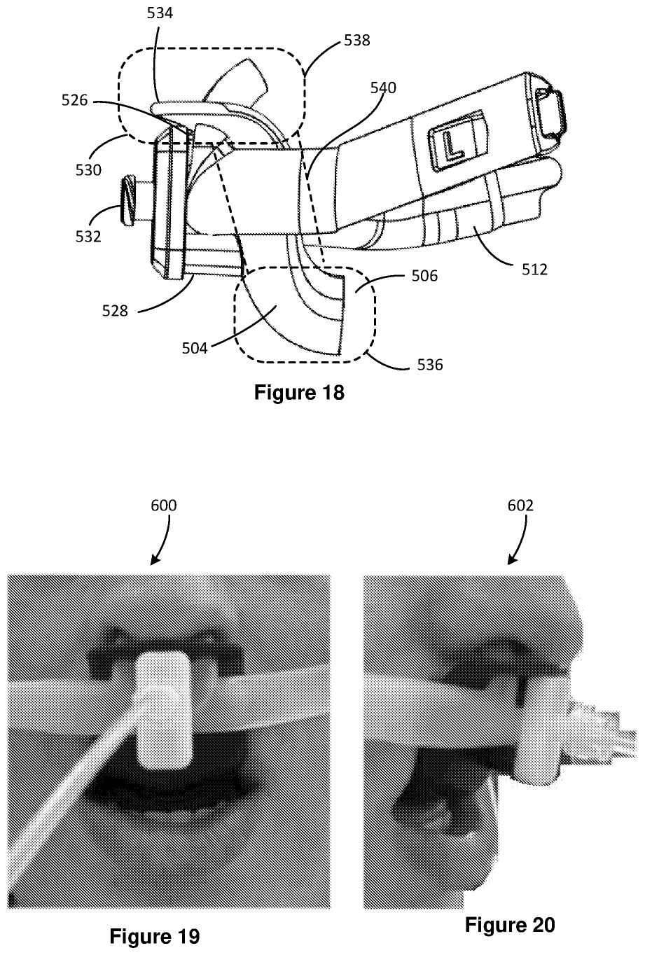

In this example, the gas collector 500 is configured to be attached to a nasal cannula 510 including a gas delivery conduit 512 (or gases delivery side member 512) and nasal prongs 514 and 516. The channel wall portion 504 includes apertures 518 and 520 enabling location of the gas collector 500 over the nasal prongs 514 and 516. The channel wall portion 504 further includes two other apertures 522 and 524 passing there through and respectively forming an oral gas inlet 522 (which may be also known as a first gas inlet 522) and a nasal gas inlet 524 (which may also be known as a second gas inlet 524). Collectively, the nasal and oral gas inlets 522, 524 may be referred to as gas inlets. In some configurations, the oral gas inlet 522 may predominantly collect gases from the patient's mouth region (for example exhaled gases from the patient's oral passages). In some configurations, the nasal gas inlet 524 may predominantly collect gases from the patient's nasal region (for example exhaled gases from the patient's nasal passages). In some configurations, the oral gas inlet 522 and/or nasal gas inlet 524 may collect gases from both the patient's nasal and oral regions. As can be best seen in FIGS. 17 and 18, the oral gas inlet 522 and nasal gas inlet 524 are connected via conduits 526 and 528 to an adapter 530 forming a gas collecting area to collect both oral and nasal gases prior to sampling and further analysis. In this embodiment, the adapter 530 includes a single outlet 532 in fluid communication with a gas analyser (not shown). In some embodiments, the adapter 530 or a portion thereof comprises a rigid structure. The adapter 530 and channel wall portion 504 are configured to locate a portion of the nasal cannula 510 therebetween such that the gas collector 500 is mounted onto the nasal cannula 510. In particular as shown in FIGS. 17 and 18, a portion of the nasal cannula 510 is sandwiched between the channel wall portion 504 and adapter 530.

In the embodiment shown in FIGS. 16 to 18, gas sampling points are shown as being combined. However, in some embodiments individual samples may be returned to two gas analysers to sample each location and determine parameters from the patient such as how the patient is breathing.

In some embodiments, the gas collecting inlets 522 and 524 may be a single inlet. In some embodiments, the channel wall portion 504 comprises an auxiliary channel that fluidly connects the first gas inlet 522 and the second gas inlet 524. Moreover, in some embodiments, the gas collector 500 may have more than one gas collecting inlet 524 for collecting nasal gases and more than one gas collecting inlet 522 for collecting oral gases.

Although in this embodiment the gas collector 500 is formed separately from and subsequently attached to a nasal cannula, in other embodiments the gas collector may be formed integrally with the nasal cannula. In such arrangements, the nasal cannula may at least partly form the channel wall portion spaced from the patient's face by appropriate spacers to form the open channel located at the upper lip of the patient.

The interface 502 may be manufactured from a variety of materials. For example, it may be manufactured from soft polymer, such as silicone, which may flex with a user's facial contours to minimise or avoid pressure points. The interface 502 may also comprise one or more materials, for example a polymer and a metal or polymers with different properties.

The spacers 506 and 508 may take the form of thickened ribs which contact the patient's face from either side of the nasal prongs 514 and 516. An open channel is created between the patient's nose, upper lip and mouth by the channel wall portion 504, which may form a thinner region of the interface 502 that is offset from the face with the help of thickened ribs 506 and 508 which provide rigidity.

Accordingly, the spacers 506 and 508 may have a thickness greater than the channel wall portion 504, and the spacers 506 and 508 may also be more rigid than the channel wall portion 504. In some embodiments of the invention, the spacers may have a stiffness greater than that of the channel wall portion (e.g. the required stiffness may be achieved via shape/configuration and/or material, in some embodiments the spacers may have a Young's modulus that is greater than that of the channel wall portion).

The channel wall portion may be formed from material or otherwise configured to enable it to flex when in contact with the patient's face. The gas collector may be able to maintain its shape when in use. In some embodiments, the gas collector is adapted to maintain a pre-form shape when in use. In a number of embodiments, the gas collector may be configured to avoid exactly matching with contours of a patient's face such that a channel with the patient's upper lip is not created when in use. In some embodiments, the gas collector may comprise a pre-form shape that has a substantially different curvature in the channel wall portion, compared to an average patient's upper lip (for example the channel wall portion is more convex in the direction away from the patient than an average patient's upper lip). In some embodiments, the gas collector is resilient and resists flexing that would conform the gas collector to the patient's face and avoid creating a channel. In other embodiments, the gas collector is configured to flex out of shape to accommodate medical scopes and other instruments which may be inserted in a patient's mouth.

It will be appreciated that the gas collector 500 shown in FIGS. 16 to 18 is non-sealing. That is, and as can be best seen in FIG. 18, the interface 500 includes a top surface 534 which sits underneath the nose of the patient when in use but does not create a seal with the nose, i.e. there is a gap between the nose and a portion of the top surface 534 to allow continuous passage of gases between the nose and atmosphere.

Similarly, and as can be best appreciated in FIG. 16, the gas collector 500 may include a mouth engagement portion 536 configured to project into a patient's mouth so as to maintain an open passage between the patient's mouth and the oral gas collecting inlet 522. In particular, the gas collector 500 is configured to project into the patient's mouth and engage only the patient's upper lip so as to pry the patient's mouth open so as to establish an open passageway for gases in the patient's mouth to exit to atmosphere and/or into channel created between the patient's nose, upper lip, mouth and channel wall portion 504.

In the embodiment shown in FIGS. 16 to 18, the mouth engagement portion includes a lower end projecting into the patient's mouth being shaped to avoid creating a seal with the patient's upper lip, i.e. there is a gap between a portion of the patient's upper lip and the mouth engagement portion to allow continuous passage of gases between the mouth and atmosphere. In other words, the mouth engagement portion is configured to project into the patient's mouth so as to maintain an open passage between the patient's mouth and the second gas collecting inlet.

In particular, in this embodiment the lower end of the mouth engagement portion has an inner surface which includes edge portions 558 and 560, best seen in FIG. 16, located, when in use, at the sides of a patient's mouth. The inner surface of the lower end of the mouth engagement portion also includes a central portion 542 (see FIG. 17) located, when in use, at the centre of the patient's mouth and spaced further from the patient's upper lip than the edge portions. In various embodiments of the invention, the inner surface of the first end of the mouth engagement portion may have a U or V shaped profile when the gas collector 500 is viewed from the perspective shown in FIG. 17. Other shapes of the mouth engagement portion may be envisaged depending on the application, for example, a C-shaped profile, or a W-shaped profile.

Whilst the top flat surface 534 depicted in FIG. 18 avoids creation of a seal with the nose, it will be appreciated that this is but one example of a more general case in which the channel wall portion 504 includes an upper end extending outwardly from the patient's face. In some embodiments, the outward extension of the upper end of the channel wall portion from the patient's face may be caused by the channel wall curving outwardly from the patient's face.

It will be appreciated from FIGS. 16 to 18 that the interface 500, and in particular the channel wall portion 504 and thickened ribs 506 and 508 form a gas flow diverter configured to direct at least some of the gases exhaled by a patient to the gas collecting inlets 522 and 524. The gas flow diverter 500 includes, as can be best seen in FIG. 18 an upper portion 538 that extends away from the patient's face, the lower portion 536 that extends towards the patient's face, an intermediate portion 540 that interconnects the lower and the upper portions. The intermediate portion 540 extends in a direction substantially parallel to the patient's face.

It can be seen from FIGS. 16 to 18 that at least the intermediate portion of the gas flow diverter has an inner surface which, in use, faces the patient's face, and substantially follows the shape of the patient's face from nasal tip (pronasale) to the upper lip region or below the upper lip region.

In a particular embodiment, the inner surface of the gas flow diverter 500, or the vertical cross-section of gas flow diverter 500 along its length and taken with respect of the patient's sagittal plane, has a substantially sigmoid shape.

Images 600 and 602 of the gas collector 500 shown in FIGS. 16 to 18, when in use, are shown in FIGS. 19 and 20. In particular, the attachment of the gas collector to a nasal cannula (for example nasal cannula 200, 1400), and the non-sealing of the upper and lower portions of the interface respectively to the patient's nasal passages and oral passage can be seen.

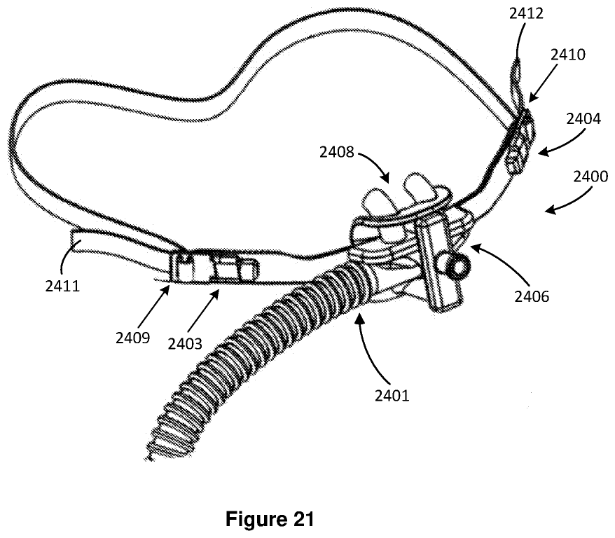

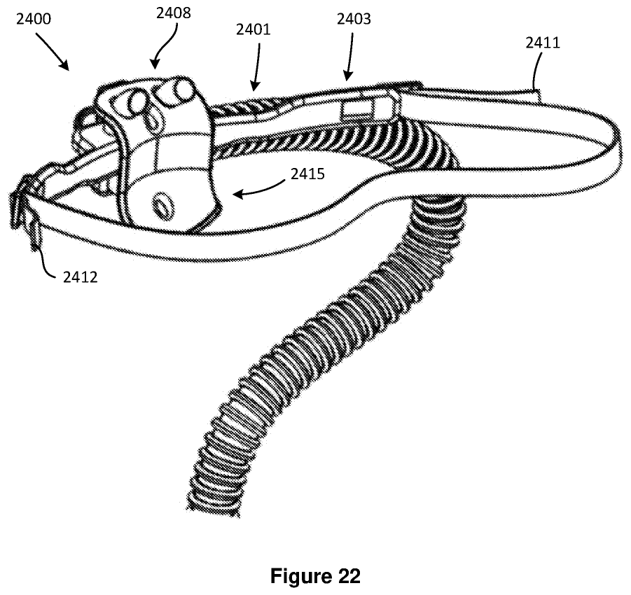

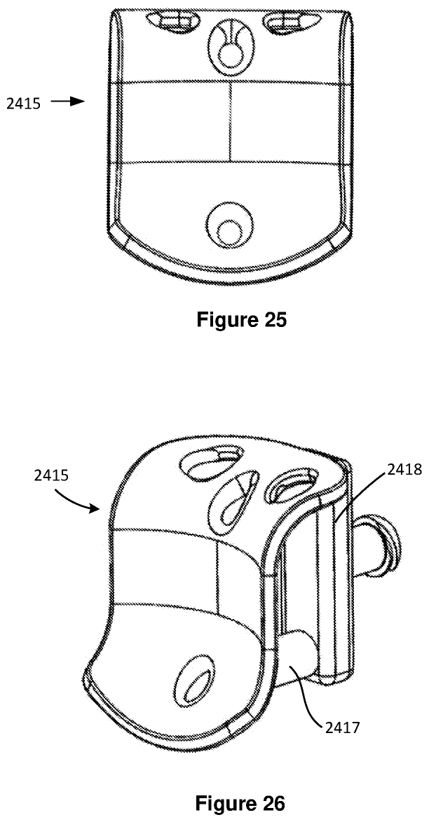

FIGS. 21 to 26 show a patient interface 2400 in the form of a nasal cannula similar to nasal cannula 30 shown in FIGS. 9 to 11. The nasal cannula 2400 includes a patient conduit 2401 configured to deliver apparatus gases (for example gas flow from a flow source) to a patient via a manifold 2406 to a delivery outlet comprising a pair of nasal prongs 2408. The pair of nasal prongs 2408 extend from the manifold 2406. The patient conduit 2401 extends from a first side of the manifold 2406. The patient conduit 2401 may extend from either the left or right side of the nasal cannula 2400. The interface 2400 further includes side arms 2403 and 2404 extending laterally from the sides of the manifold 2406. The side arms 2403 and 2404 respectively include ends 2409 and 2410 configured for connection to headstrap ends 2411 and 2412.

Unlike the embodiment shown in FIGS. 5 to 8, the gases delivery side member 1401 does not include a collapsible portion configured to move from the normally open to a collapsed configuration.

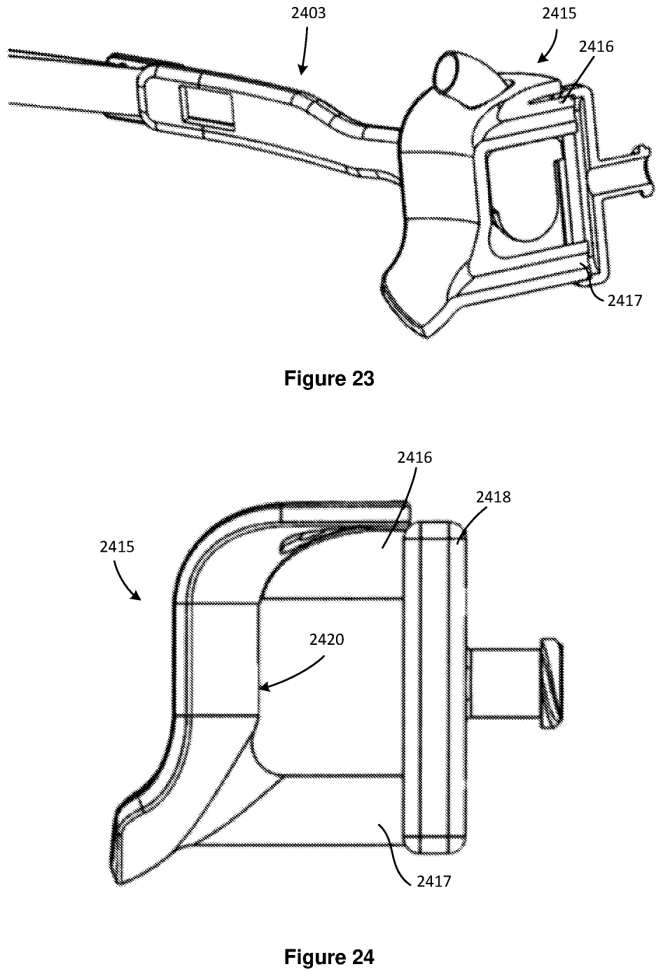

The gas collector 2415 may be separately formed form and removably attachable to the nasal cannula 2400. For example, as can be best seen in FIGS. 23 and 24, the adaptor 2418 is engageable with the body 2420 of the gas collector 2415 to retain the gas collector 2415 on the nasal cannula 2400. When engaged, one or more flow passages in the adapter 2418 is in fluid communication with one or more passages of conduits 2416 and 2417. In other embodiments the gas collector 2415 is integrally formed with the nasal cannula. Features of the gas collector 2415 are shown in FIGS. 23 to 26. In many respects, the gas collector 2415 is substantially identical to the gas collector 500 shown in FIGS. 16 to 20. In some embodiments the gas collector 2415 is integrated with, rather than being attached to conduits 2416 and 2417 which in turn are integrated with adapter 2418 forming a gas collecting area to collect both oral and nasal gases prior to sampling and further analysis.

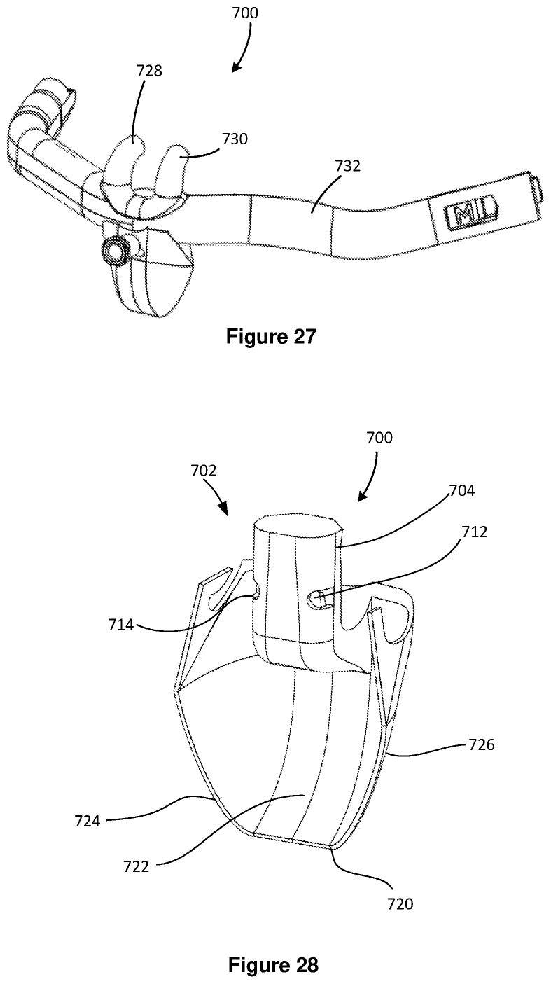

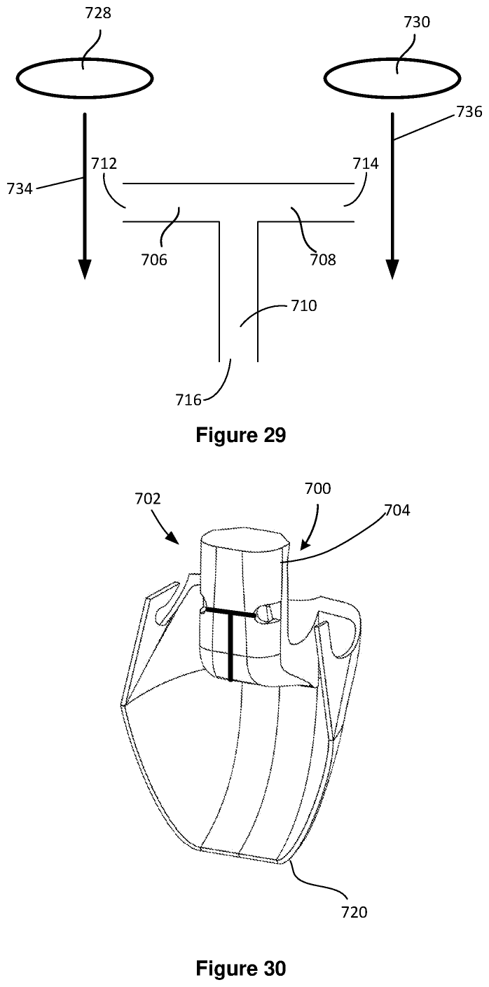

FIGS. 27 to 35 depict a further embodiment of a gas collector for collecting gases exhaled by a patient from their nasal passages and oral passage. Embodiments shown in these figures form a second channel type, as shown in FIG. 15. In this embodiment, a gas collector 700 includes an interface 702 configured to form a plurality of channels at an upper lip of the patient, the channels having open ends respectively in fluid communication with the patient's nasal passages and oral passage. The interface 702 includes a spacer 704 which, in use, rests against the patient's upper lip. In an example, a portion the nasal cannula 732 may contact a portion of the patient's upper lip in use, similar to the first channel type as shown in FIG. 13. In such an example, the gas collector may form a combination of first and second channel types as shown in FIGS. 13 and 15. One or more nasal and oral gas collecting conduits 706 to 710, shown schematically in FIG. 29, are formed within the spacer 704. Arrows 734, 736 illustrate a flow direction of redirected supply gases from the nasal prongs 728, 730. Each nasal gas collecting conduit 706 and 708 has a nasal gas collecting inlet, respectively referenced 712 and 714 in fluid communication with the patient's nasal passages, for delivering gases exhaled by a patient from their nasal passages to an outlet. The oral gas collecting conduit 710 formed within the spacer has an oral gas collecting inlet 716 in fluid communication with the patient's oral passage.

The gas collector also includes a gas flow diverter 720, this embodiment having the form of a concave scoop, configured to direct at least some of the gases exhaled by the patient from their oral passage to the oral gas collecting inlet. The gas flow diverter 720 may be integrated with or attachable to the spacer 704.

The issue of dilution of nasally and orally exhaled gases are addressed in a number of ways. For orally exhaled gases, the gas flow diverter limits exhaled gases from escaping or mixing easily with the atmosphere. In that regard, the gas flow diverter is in the form of a concave scoop having a central portion 722 that extends towards the patient's face as well as side portions 724 and 726 which collectively act to minimise exhaled gas from escaping. Additionally, for nasally and orally exhaled air the inlets 712 to 716 are positioned to maximise volume of exhaled breath collection and minimise entry of supplied air from nasal prongs 728 and 730 forming part of a nasal cannula 732 to which the gas collector 700 is attached.

The issue of dilution is exacerbated in NHF applications. The redirected gases supplied to the patient via the nasal prongs 728 and 730 can dilute expired air as well as stopping orally expired air from entering the gas collecting inlets 712 to 716. One measure used to address this issue in the embodiment shown in FIGS. 27 to 35 is for the nasal gas collecting inlets 712 and 714 to be located proximate the patient's nasal passages, when in use. Additionally, the nasal gas collecting inlets 712, 714 are arranged in a position where they are transverse to the length of the created channel. This position minimizes the dynamic pressure with which gases enter the nasal gas collecting conduits 706, 708. The nasal gas collecting inlets 712 and 714 may therefore be arranged in one or more of the following positions: a first position wherein the nasal gas collecting inlets 712, 714 are substantially parallel to a length of the channel and facing towards the gas flow from the patient's nasal region, a second position wherein the nasal gas collecting inlets 712, 714 are substantially transverse to a length of the channel; and a third position that is between the first and second positions. Nasal gas collecting inlets 712, 714 that face towards a gas flow from the patient's nasal region would create a region of high pressure within the nasal gas collecting conduits 706, 708, which in the embodiment where they are connected with the oral gas collecting conduit 716, increase the resistance to flow in the oral gas collecting inlet 716 and conduit 710, thereby making it more difficult to collect gases from the patient's oral region.

A further measure is for the oral gas inlet 716 to be located, when in use, at the bottom of the spacer 704 proximate the patient's mouth. As can be seen in FIG. 29, this enables the oral gas collecting conduit 710 to form a path leading to a junction with the nasal gas collecting conduits 706 and 708. The concave scoop form of the gas flow diverter 720 assists in guiding the orally expired gas to the oral gas collecting inlet 716. An advantage of having such a junction is that there is substantial capture of orally expired gases that may not be washed out by the redirected supply gases from the nasal prongs 728 and 730.

The issue of capturing exhaled gases as the patient switches between oral and nasal exhalation is further addressed by providing distinct gas collecting inlets for the collection of exhaled gases placed in the junction depicted schematically in FIG. 29. The one or more nasal gas sampling conduits and the oral gas sampling conduit are configured so that a flow rate in the one or more nasal gas sampling conduit is a percentage of a total flow rate in the one or more nasal gas sampling conduits and the oral gas sampling conduit, where said percentage is within a predetermined range. This total flow rate includes a calculated total flow rate of a flow rate in the one or more nasal gas sampling conduits and a flow rate in the oral gas sampling conduit (i.e. mathematically adding up their respective flow rates), and a flow rate of a combined flow when the flow in the one or more nasal gas sampling conduits and the flow in the oral gas sampling conduit are combined, e.g. at a junction.

In some configurations where there is a plurality of nasal gas sampling conduits, the plurality of nasal gas sampling conduits and the oral gas sampling conduit are configured so that a combined flow rate in the plurality of nasal gas sampling conduits is a percentage of a total flow rate in the plurality of nasal gas sampling conduits and the oral gas sampling conduit, where the percentage is within a predetermined range. In some configurations, there is a plurality of oral gas sampling conduits. In such configurations, the one or more nasal gas sampling conduits and the plurality of oral gas sampling conduits are configured so that a flow rate in the one or more nasal gas sampling conduits is a percentage of a total flow rate in the one or more nasal gas sampling conduits and the plurality of oral gas sampling conduits, where the percentage is within a predetermined range. The predetermined range may be from about 1% to about 99% (for example, the flow rate in one or more nasal gas sampling conduits is 1% of the total flow rate in the one or more nasal gas sampling conduits and the oral gas sampling conduit), and preferably from about 5% to about 95%, and even more preferably from about 20% to about 80%. In some embodiments, the predetermined range is from about 45% to about 55%. In some embodiments, this ratio may be about 50% such that the flow rates in the one or more nasal gas sampling conduits and one or more oral gas sampling conduits are substantially balanced.

In some embodiments, the relative positions of the nasal and/or oral gas inlet openings may be configured to achieve the abovementioned percentage in the predetermined range. For example, as shown in FIG. 30, the nasal gas inlet openings are positioned perpendicular to a flow direction through the channel, from the patient's nasal region to the patient's oral region, while the oral gas inlet opening is positioned to face the patient's oral region and is substantially parallel to the flow direction through the channel, from the patient's nasal region to the patient's oral region.

In some embodiments, to achieve the abovementioned percentage in the predetermined range, the nasal gas sampling conduits 708 and 706 and the oral gas sampling conduit 710 may be configured so that the ratio of resistance to flow (RTF) in one or more nasal gas sampling conduits and the oral gas sampling conduit is within a predetermined range. In some embodiments, the nasal gas sampling conduits 708 and 706 and the oral gas sampling conduit 710 may be configured so that the ratio of a combined RTF in the one or more nasal gas sampling conduits and the RTF in the oral gas sampling conduit is within a predetermined range.

In one or more embodiments, the nasal gas sampling conduits and oral gas sampling conduits may be configured so that the RTF in these respective conduits is substantially equalised (i.e. substantially the same). Having substantially equalised RTF in the respective conduits may not result in substantially balanced flow rates in the nasal gas sampling conduits and oral gas sampling conduits.

In one or more embodiments, the RTF in one or more of the nasal gas sampling conduits (or combined RTF in the one or more nasal gas sampling conduits) may be greater than the RTF in the oral gas sampling conduit by a predetermined amount. This predetermined amount may be based on the abovementioned percentage in the predetermined range of the flow rate in one or more nasal gas sampling conduits and a total flow rate in the one or more nasal gas sampling conduits and the oral gas sampling conduit. For example, the RTF in one or more of the nasal gas sampling conduits (or combined RTF in the one or more nasal gas sampling conduits) may be about 1%, about 2%, about 3%, about 4%, about 5%, about 10%, about 20%, about 30%, about 40%, about 50%, about 60%, about 70%, about 80%, about 90%, about 100% or more than about 100% greater than the RTF in the oral gas sampling conduit. In some embodiments, the RTF in one or more of the nasal gas sampling conduits (or combined RTF in the one or more nasal gas sampling conduits) may be about 200%, about 300%, about 400% or more than about 400% greater than the RTF in the oral gas sampling conduit. This may be beneficial when the patient is predominantly supplied with nasal high flow gas from the nasal prong 728 and 730 of the nasal cannula 732 and is predominantly mouth breathing.

In other embodiments, the RTF in the nasal gas sampling conduits may be less than the RTF in the oral gas sampling conduit. In other words, the RTF in the oral gas sampling conduit may be greater than the RTF in one or more of the nasal gas sampling conduits (or combined RTF in the one or more nasal gas sampling conduits) by a predetermined amount. For example, the RTF in the oral gas sampling conduit may be about 1%, about 2%, about 3%, about 4%, about 5%, about 10%, about 20%, about 30%, about 40%, about 50%, about 60%, about 70%, about 80%, about 90%, about 100% or more than about 100% greater than the RTF in the one or more of the nasal gas sampling conduits (or combined RTF in the one or more nasal gas sampling conduits). This may be beneficial when the patient is predominantly supplied with nasal low flow gas from the nasal prong 728 and 730 of the nasal canula 732 and is predominantly nose breathing.

Configuration of the nasal and oral gas sampling conduits to achieve a ratio of RTF between the nasal gas sampling conduits and the oral gas sampling conduit may be achieved by configuring one or more of parameters (e.g. cross-sectional area, shape, length, surface roughness etc) of the conduits or a flow path in the conduits. In some embodiments, the oral gas sampling conduit comprises a more tortuous flow path and/or comprises a longer flow path to a junction (for example where the flow paths in the conduits combine) compared to the one or more nasal gas sampling conduits. In some embodiments, a cross-sectional shape and/or area of a flow path in one nasal gas sampling conduit is different from a cross-sectional shape and/or area of a flow path of another nasal gas sampling conduit and/or the oral gas sampling conduit.

In some embodiments, an internal surface of the one or more nasal sampling conduits exposed to a gas flow is different from an internal surface of the oral gas sampling conduit exposed to a gas flow, for example, the internal surface of the one or more nasal sampling conduits comprises a surface roughness greater than an internal surface of the oral gas sampling conduit (thereby causing the one or more nasal sampling conduits to have a higher RTF than the oral sampling conduit). In some embodiments, the RTF in the one or more nasal gas sampling conduits and/or the oral gas sampling conduit may be variable. In some embodiments, such variable RTF is adjustable. In some embodiments, the cross-sectional area and/or shape of a portion of one or more sampling conduits may be adjustable, for example, by providing a valve that may be controlled to alter a parameter of the flow path through that portion. The valve may be controlled automatically or manually e.g. by a rotary dial. In some embodiments, the portion may be configured to be collapsible, for example being less rigid compared to the rest of the conduit, such that a force acting on the portion may alter a parameter of the flow path through that portion.

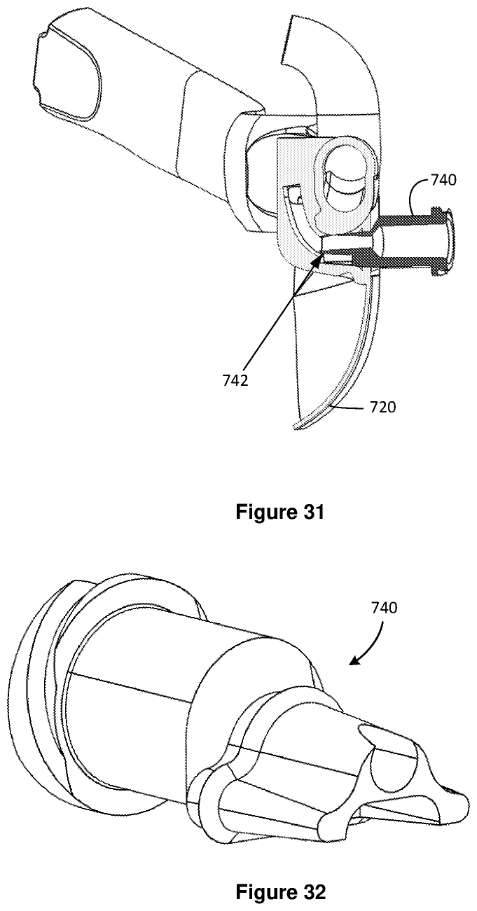

The RTF of the oral gas sampling conduit 710 may be increased with the use of a connector 740 (shown separately in FIG. 32) and shown in FIG. 31, connected to an outlet to the gas collection area at the junction of the nasal and oral gas collecting conduits 706 to 710. As seen in the cross-section of the gas collector depicted in FIG. 31, the connector 740 (which may be a luer type connector) includes a ledge 742 which when inserted into the outlet will form a tortuous path for the orally expired air. Accordingly, there is no or minimal bias in flows which favours expired nasal gas and redirected supply gas entering the nasal gas inlets 712 and 714 over orally expired gas entering the oral gas inlet 716. This means in the case where the patient is mouth breathing, the redirected flows that enter the nasal gas inlets will not wash out the orally expired flow entering the oral gas inlet. That is, the breath that exits the mouth may also be captured.

In the images shown in FIGS. 31 and 32, the connector 740 is a separate component. However, it will be appreciated that in other embodiments the connector may be integral with the remainder of the gas collector.

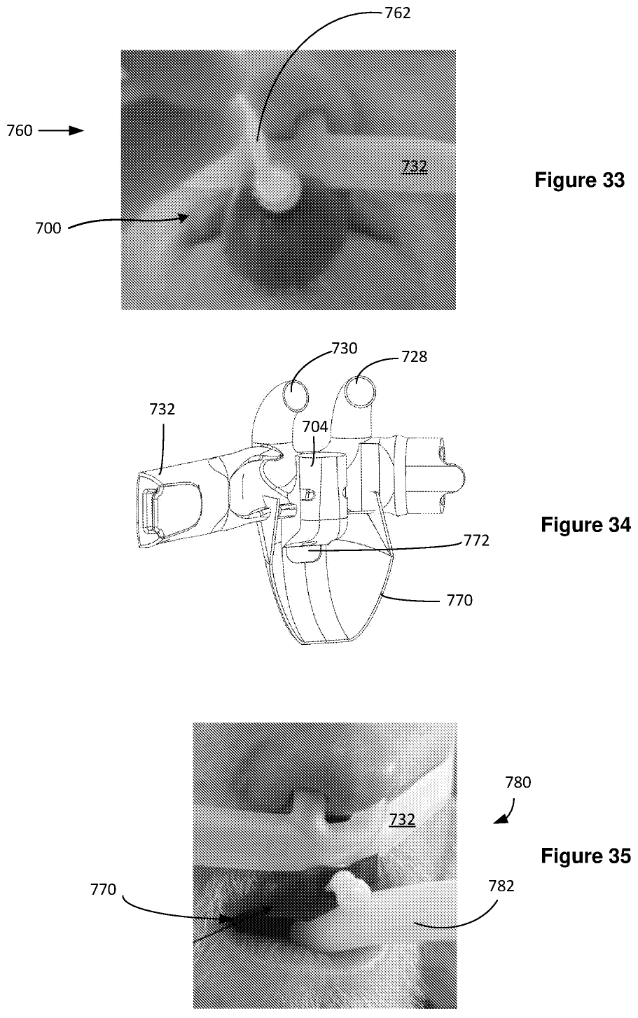

FIG. 33 shows an image 760 of the gas collector 700 when attached to the nasal cannula 732, where a tube 762 couples the connector 740 to a gas analyser (not shown).

In a variation, the gas flow diverter may include a window to aid orally exhaled air to enter the oral gas collecting inlet 716. As shown in FIG. 34, the gas flow diverter 770 includes a window 772 located proximate the spacer 704 and the oral gas inlet 716. It will be appreciated that the window 772 may be of any desirable shape. The window creates a pathway of negligible resistance for the orally exhaled gases to flow through. When this occurs, the oral gases will be travelling over the oral gas collecting inlet 716.

Both embodiments of the gas flow diverter shown may be made of material or structure enabling the gas flow diverter to be flexible and to fold or make way for any oral instruments that need to be inserted into the oral cavity of the patient. Such an example is shown in the image 780 of FIG. 35 where it can be seen that the gas flow diverter 770 is bent to be insertable into the patient's mouth and to enable an oral conduit 782 or any other oral instrument to be inserted into the patient's mouth.

In some embodiments of the gas collector, it will be understood that any part of the gas collector may be flexible to enable medical instruments to be inserted into the patient's nose and/or mouth as required.

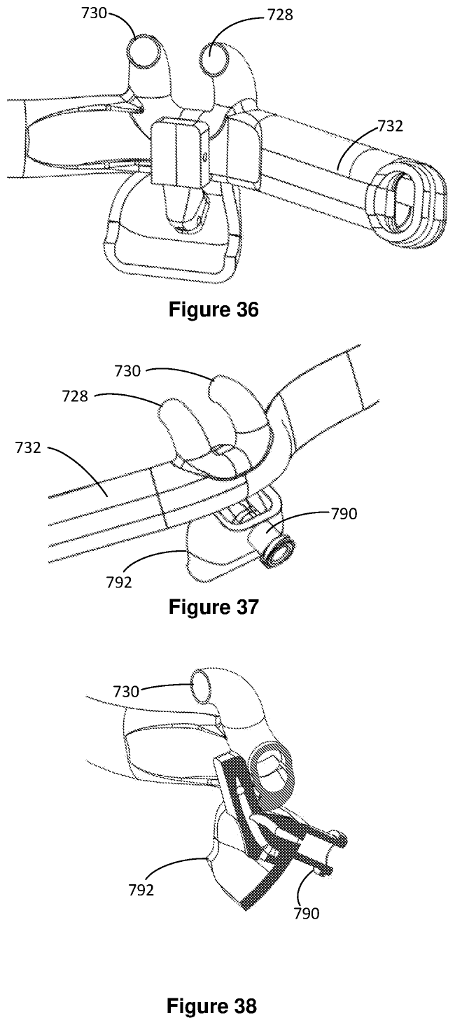

FIGS. 36 to 38 depict a variation to the embodiments shown in FIGS. 16 to 20 and 27 to 35. In this embodiment, a connector 790 (e.g. a luer connector) is moulded as one piece with the gas flow diverter 792. The open channels created in this variation are a combination of those shown in FIGS. 13 and 15. That is, spacer 422 of FIG. 15 in the middle with spacers 418 and 420 of FIG. 13 on either end.

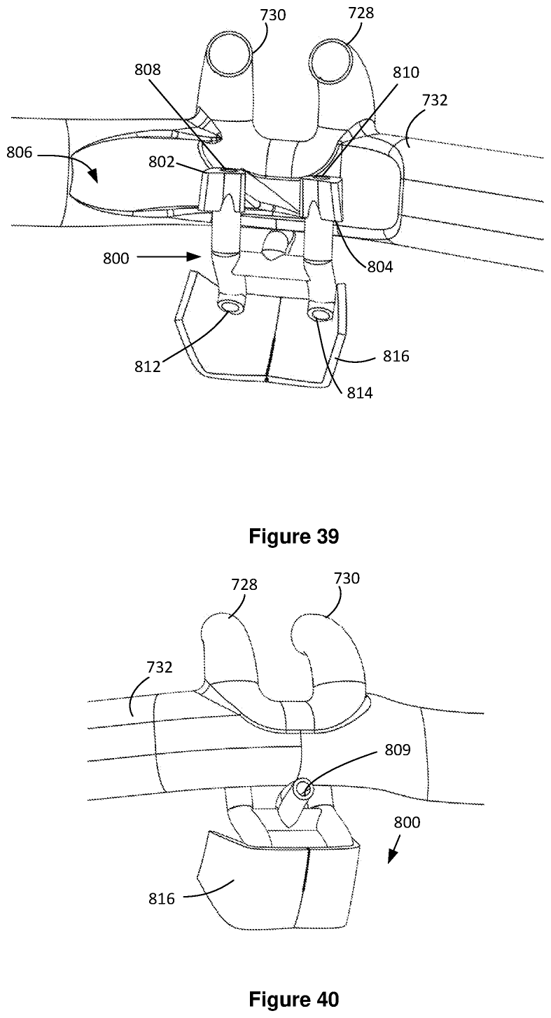

FIGS. 39 and 40 depict an embodiment of the gas collector corresponding to the third type of open channel structure depicted in FIG. 14. In that regard, the gas collector 800 shown in these figures includes two spacers 802 and 804 positioned at the upper lip of the patient and, when in use, on either side of the patient's philtrum region to space an inner wall 806 of the nasal cannula 732 from the upper lip of the patient. In this way, multiple channels are formed at the upper lip of the patient and the nasal cannula 732 at least partially forms a channel wall portion spaced from the patient's upper lip by the spacers 802 and 804. The spacers 802 and 804 respectively include nasal gas collecting inlets 808 and 810. A tubing arrangement is provided to interconnect the nasal gas collecting inlets 808 and 810 and oral gas inlets 812 and 814 so that the collected gases are supplied at a localised gas collector area. In some embodiments, the gas inlets 808 and 810 may be perpendicular to the flow of gases through the channel as described elsewhere in the specification. For example, the gas inlets 808 and 810 may be arranged in positions as described in the embodiment of FIGS. 27 to 35. An outlet 809 is provided at that local gas collector area in order to supply the collected gases to a sample analyser (not shown) for analysis. A gas flow diverter 816 in the form of a concave scoop is fitted around the oral gas inlets 812 and 814 and acts to funnel or direct at least some of the gases exhaled by a patient from their oral passage to the oral gas collecting inlets 812 and 814.

In this arrangement, the nasal gas collecting inlets and nasal gas collecting conduits are in-line with the nasal prongs 728 and 730. The inlet diameters for the nasal gas collecting inlets may be configured to be smaller than those of the oral gas collecting inlets. In some embodiments, the nasal gas conduit or a portion thereof has a smaller cross-sectional diameter than the oral gas conduit or a portion thereof. In this way, the RTF of the pathways for the nasal and oral gases can be balanced to a desirable state. This configuration may be useful in particular for low flow respiratory therapy.

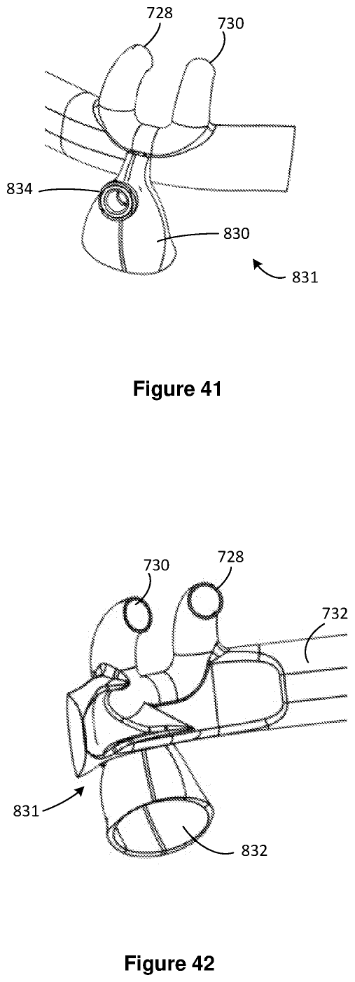

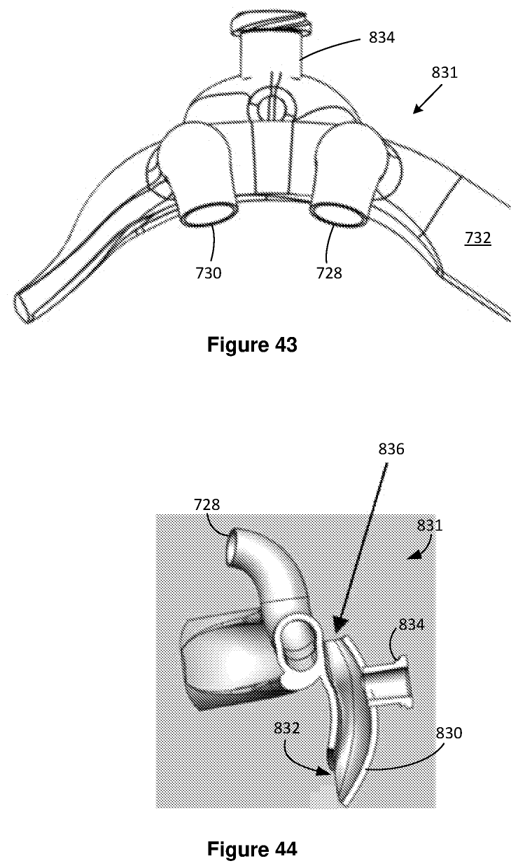

FIGS. 41 to 44 depict a further embodiment of the gas collector 831. In this embodiment the gas collector is situated on the non-patient contacting portion of the nasal cannula 732. In some embodiments, the gas connector may be situated on the patient contacting portion of the nasal cannula 732. In this embodiment, the gas collector comprises a mouth engagement portion 830 and is integrally formed with the nasal cannula 732. In some embodiments, the gas collector may be removably attached to the nasal cannula 732 by methods described elsewhere in this specification. The mouth engagement portion is in the form a scoop including an elongate aperture 832. The scoop may project into or sit proximate to the patient's mouth when in use to capture gases at the patient, for example, gases exhaled by the patient from their mouth, and deliver said gases to a gas collection area. An outlet 834 is formed at the rear of the gas collector to provide the collected gases to a gas analyser (not shown).

The mouth engagement portion 830 sits proximate to or in the patient's mouth. Gases exiting the patient's mouth typically travel slower than gases exiting the patient's nostrils due to the mouth opening having a larger cross-sectional area compared to the nostrils. Gases exiting the nose may be at higher velocity due to the combined effect of smaller cross sectional area of the nostrils and/or the redirected supply flow from the prongs 728 and 730. Thus, the oral gas aperture 832 may be configured to position closer to the mouth relative to nasal gas inlet 836 when the gas collector is positioned on the patient's face. The oral gas aperture 832 would also comprise a cross-sectional area larger than a cross-sectional area of the nasal gas inlet 836. In the embodiment shown, the nasal gas inlet 836 sits between and inferior to the base of the prongs 728 and 730, adjacent to a non-patient contacting portion of the nasal cannula 732. The nasal gas inlet 836 may however be positioned at other locations.

The nasal and oral gas inlets may be positioned to maximise collection of gases at the nasal and oral regions of the patient. For example, in high flow applications, a high flow of gas from the nasal cannula 732 may be redirected into the nasal gas collecting inlet. This high flow of gas may have a flow rate greater than a flow rate of gas from the patient's oral region. Hence, it would be beneficial for the nasal inlet to be positioned distal from the patient's nasal region and/or the prongs of the nasal cannula 732, and the oral gas collecting inlet to be positioned proximal the patient's oral region. Additionally or alternatively, it would be beneficial for the nasal gas inlet (or any portion of a conduit downstream of the nasal gas inlet) to comprise a cross-sectional area less than the oral gas collecting inlet (or any portion of a conduit downstream of the oral gas inlet).

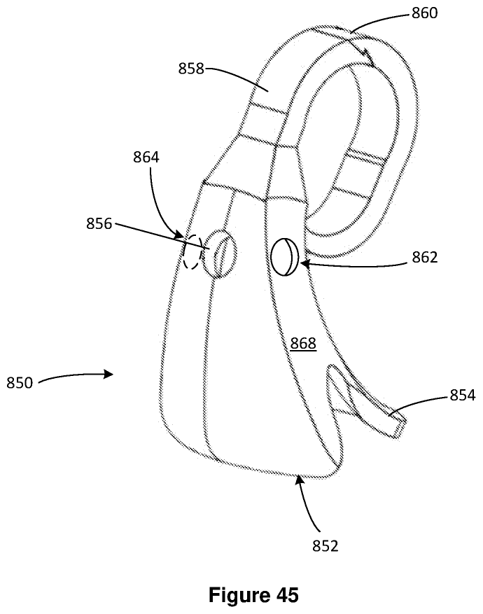

FIG. 45 shows a gas flow diverter 850 similar in form and function to the gas scoop 830, with some notable exceptions. The gas flow diverter 850 is also in the form of a scoop 868 having a large opening inlet funnel 852 located, when in use, proximate a patient's mouth. The gas flow diverter 850 further includes a top lip 854 which, when in use, rests underneath the patient's upper lip. This ensures that an open channel is created between the patient's mouth and the inlet funnel 852. Keeping the mouth open promotes mouth breathing and exhalation through the mouth which helps to ensure a target gas trace is collected. The gas flow diverter 850 further includes nasal ports 862 and 864, located on opposing sides of the scoop 868 so that each nasal port is proximate one of the patient's nasal passages.

Consistent with previous embodiments, the embodiment shown in FIG. 45, is non-sealing, has a low profile and does not fully cover the mouth in a way that a mask does. The unintrusive nature of the device shown in FIG. 45 allows for insertion of other devices through the mouth, such as a laryngoscope. Exhaled gases are diverted through the inlet funnel 852 and through to an outlet port 856 which connects to a sampling conduit (not shown). The conduit then transports the sample gases to a gas analyser (typically known as a side stream capnography when the target gas is CO₂). Additionally, or alternatively, the conduit may comprise the gas analyser.

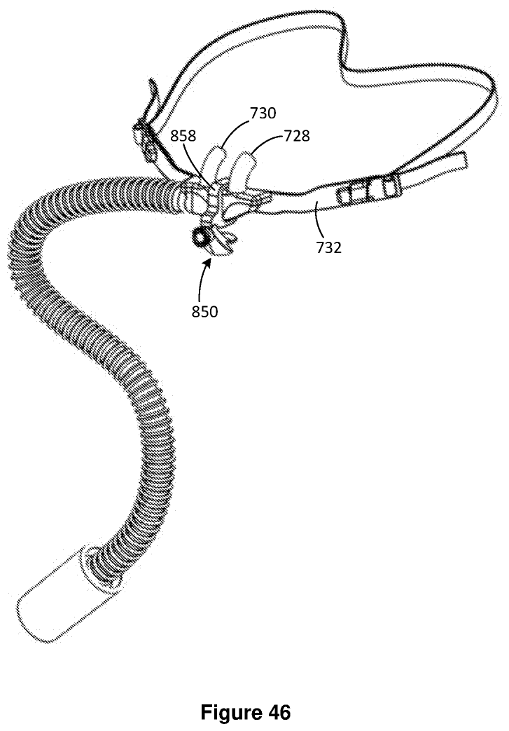

The arrangement shown in FIG. 45 is formed separately from and removably attachable with a nasal cannula, for example nasal cannula 30 as shown in FIGS. 9 to 11. In that regard, the gas flow diverter 850 includes an attachment ring 858 and corresponding latch 860 to enable mounting of the gas flow diverter 850 around the nasal cannula. In such an arrangement, shown in in FIG. 46, the attachment ring and latch attach between the nasal prongs 728 and 730 (or nasal prongs 33,34 of nasal cannula 30 as shown in FIGS. 9 to 11).

Ideally, the attachment ring 858 will have a profile to closely fit around the base of the cannula prongs, creating a secure attachment. The gas flow diverter 850 preferably mounts, directly or indirectly, to the rigid manifold part of the nasal cannula which provides a stable mounting base to improve stability and consistency in sampling. Any suitable shape of the attachment ring may be used to fit with various patient interfaces.

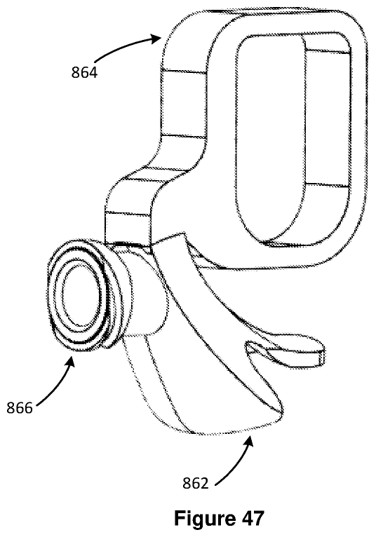

A further embodiment of the gas flow diverter 850 is shown in FIG. 47. In this embodiment, the gas flow diverter 862 has a similar form and function to the gas flow diverter 850. However, an attachment ring 864 having a shape adapted to suit a different patient interface is depicted, as well as a connector 866 being inserted into the outlet port.

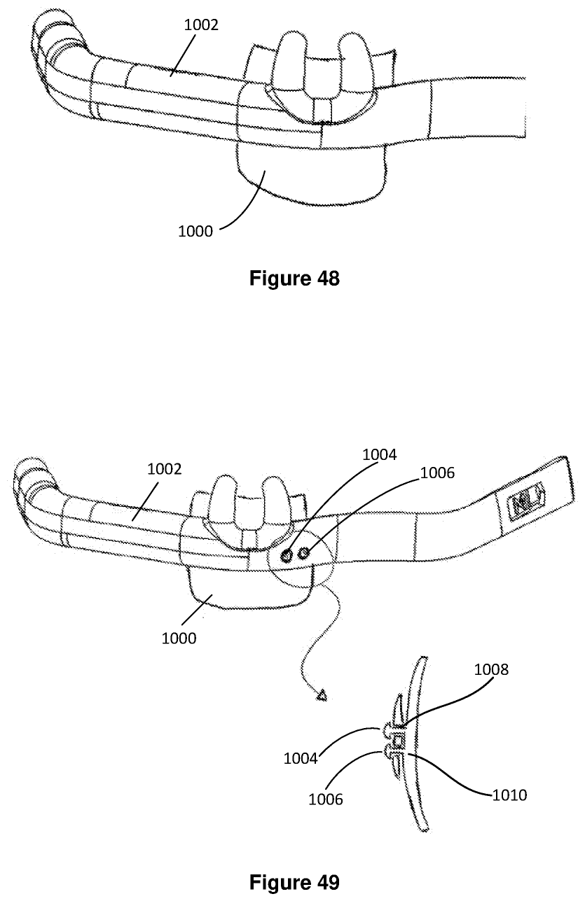

The various embodiments of the gas collector previously described and shown in previous drawings may be attached to a nasal cannula in any one of a number of ways. For example, as shown in FIG. 48, an exemplary gas collector 1000 may be attached to a nasal cannula 1002 by means of adhesives, such as glue, sticky tape or any other type of adhesive that can be applied to facing surfaces of the gas collector 1000 and the nasal cannula 1002. Alternatively, the gas collector 1000 may be attached to the nasal cannula 1002 by other means of bonding for example, bonding with solvents or RF welding or ultrasonic welding.

Hook-and-loop fasteners or like sticky or adhesive patches may also be attached to the patient's face. The opposing side of the sticky patch on the patient's face may have hook or loop material. The gas collector interface may have hook or loop material on winglets that mate with the patch on the patient's face. This may allow a clinician to position the sampling interface correctly on the patient's face. In addition, hook and loop patches may also be used on the nasal cannula or other sampling interface to connect to the gas collector. This also allows a clinician to adjust the position of the gas collector.

As shown in FIG. 49, another attachment method may include the provision of push through buttons, otherwise known as barbs, on one or other of the gas collector 1000 and the nasal cannula 1002. The push through buttons 1004 and 1006 engage in corresponding apertures 1008 and 1010 in order to secure the gas collector to the nasal cannula. The use of multiple barbs or buttons and multiple corresponding apertures can be used to constrain the gas collector from relative rotation with respect to the nasal cannula.

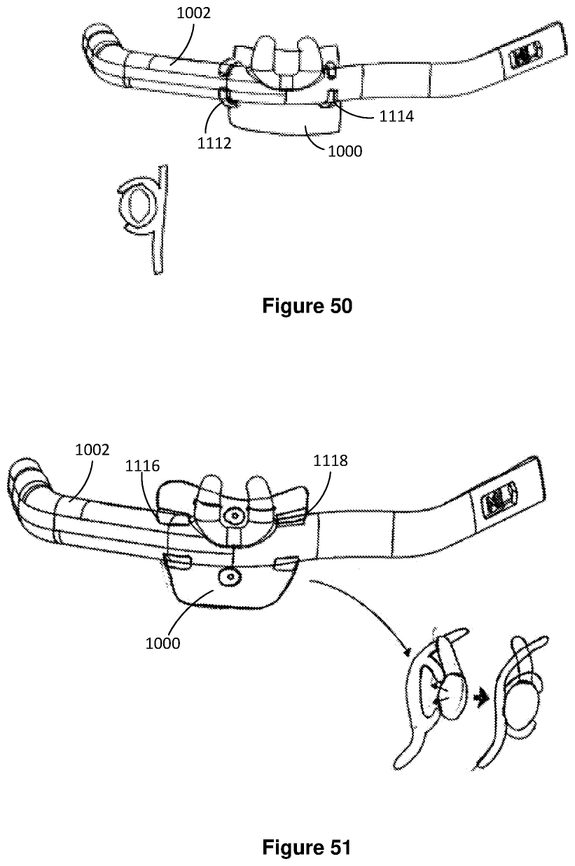

A further attachment method is shown in FIG. 50. In this example, clips 1112 and 1114, for example having a C-structure, protruding from the gas collector 1000 are provided to enable securement of the gas collector 1000 to the nasal cannula 1002. The clips may be formed from a flexible material to allow the nasal cannula 1002 to be deformed and pushed through the break in the C-structure of the clip. The nasal cannula 1002 will return to its original geometry once inserted into the C-structure clips 1112 and 1114. A further such attachment method involving C-structure clips is shown in FIG. 51 in which insertion of the nasal cannula 1002 through the break in the C-structure clips 1116 and 1118 causes elastic deformation of the c-structure clips and retention of the nasal cannula. Typically, the nasal cannula 1002 is hollow and made from a soft/flexible material. As such, the nasal cannula 1002 may be compressed to facilitate insertion into the C-structure clips 1116, 1118. Once fully inserted into the C-structure clips 1116, 1118, the nasal cannula 1002 returns to its original shape.

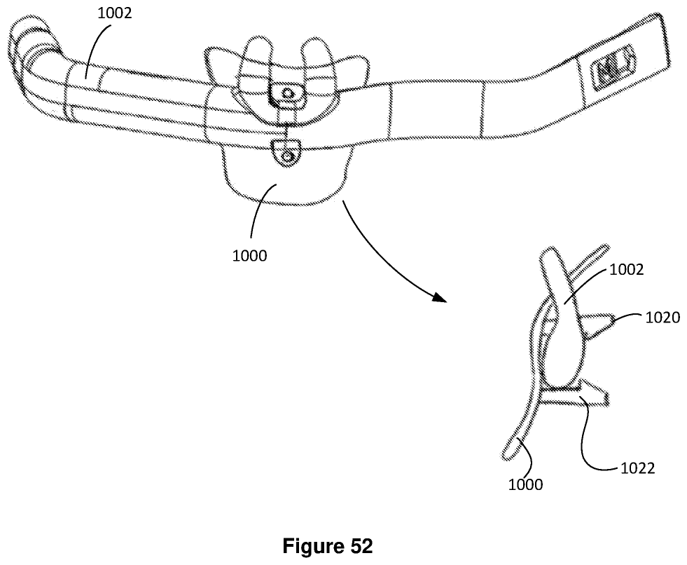

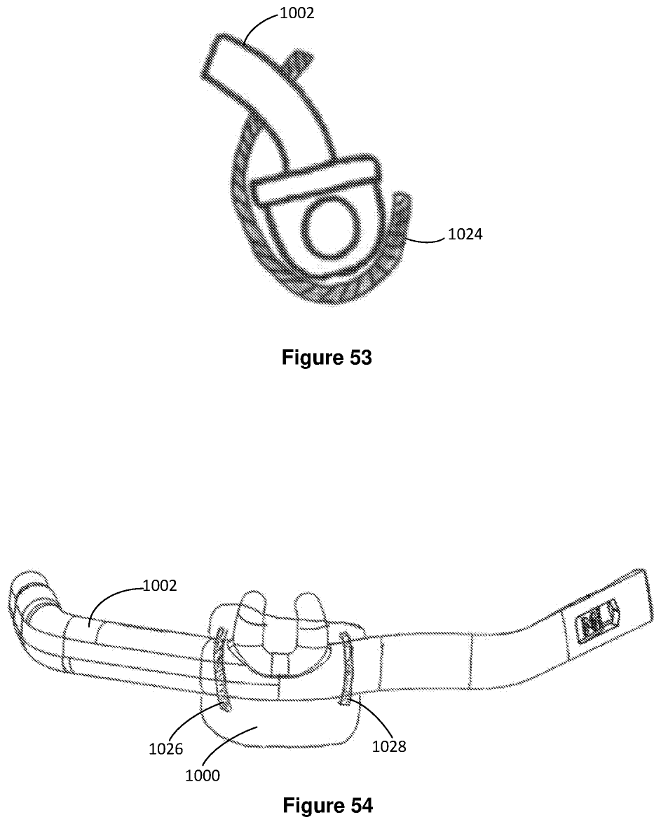

A further attachment arrangement is depicted in FIG. 52. In this figure, gas collecting inlets formed in the gas collector 1000 are provided with integral “barbs” 1020 and 1022 that act as a clip to lock the nasal cannula 1002 in place in a similar fashion as the clip concepts explained in previous figures. In a further embodiment, shown in FIG. 53, spring loaded clips 1024 may be provided on the gas collector 1000. A clinician can open the clip in order to insert the nasal cannula 1002. The clip may be spring loaded such that it will resume the shape as shown in the drawing after elastic deformation. The clip 1024 will tightly wrap around the manifold of the nasal cannula 1002.

In yet another attachment method shown in FIG. 54, straps 1026 and 1028 may be provided on the gas collector 1000 to enable the nasal cannula 1002 to be secured to the gas collector 1000. The straps can be separate paths or integral to the gas collector 1000. If separate, the straps may be secured over the gas collector 1000 and the nasal cannula 1002 to secure them together.

Straps that are integral to the gas collector 1000 may take the form of slits cut into extended winglets projecting from sides of the gas collector 1000. The gas collector 1000 will be slipped over the nasal cannula 1002.

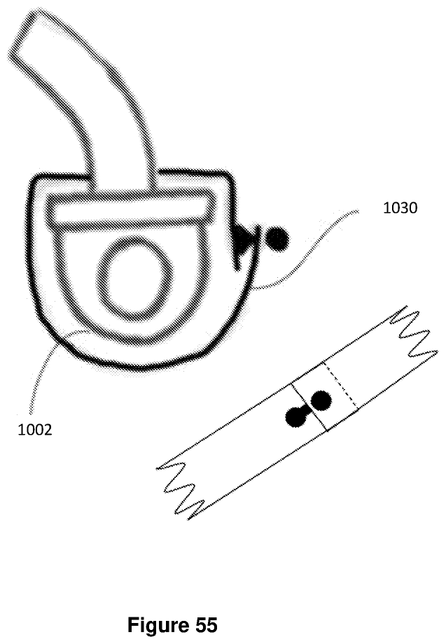

Yet another attachment method is depicted in FIG. 55. In this case, a length of strap material 1030 may be looped around the nasal cannula 1002. A terminating portion of the strap material 1030 may have a buckle, zip tie, pin connector or like which either permanently or impermanently ties the gas collector 1000 (not shown) to the nasal cannula 1002.

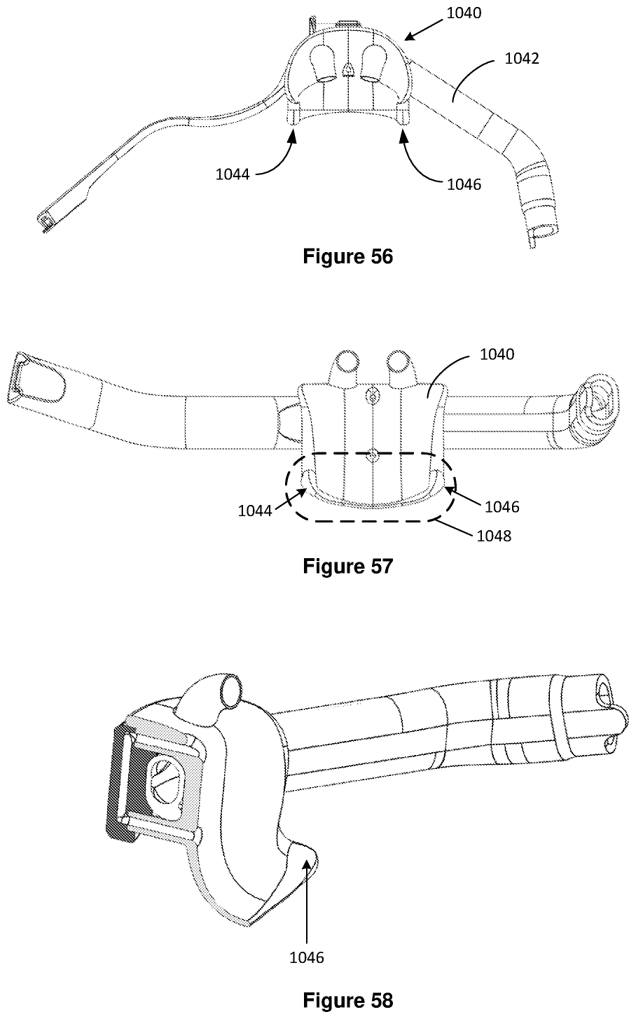

FIGS. 56 to 59 depict a gas sampler 1040 attached to a nasal cannula 1042. In this embodiment, the edges 1044 and 1046 of a U-channel section 1048 of the gas collector 1040, that is, the mouth engagement portion configured to project into the patient's mouth so as to maintain an open passage between the patient's mouth and the oral gas collecting inlet, in one embodiment can have a raised profile which will cause an intermediate portion of the U-channel section 1048 to be further away from the patient's top lip. This creates a deeper opening/orifice channel at the mouth engagement portion, which advantageously makes the opening/orifice more difficult to occlude, for example, by saliva or by a patient's teeth or tongue.

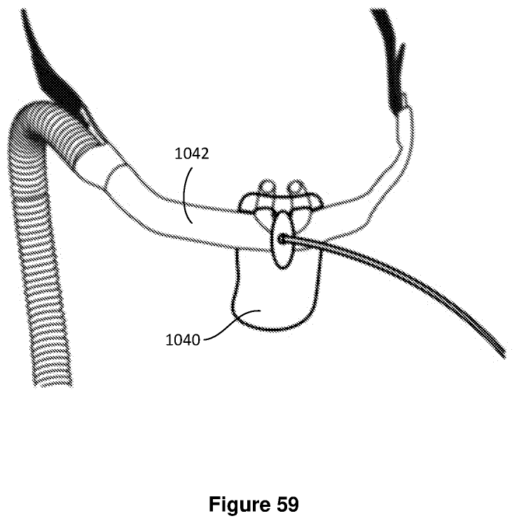

The gas collector 1040, here represented in the form of a sampling scoop, may be transparent. As shown in FIG. 59, the gas collector body 1040 may be made of transparent material such as Poly Methyl Metharylate (PMMA), Poly Carbonate (PC), silicone, thermoplastic elastomer (TPE) (e.g. Styrene Ethylene Butylene Styrene (SEBS)). Providing a transparent body of this nature will enable a healthcare worker to see a patient's face which will be useful to ensure proper placement of nasal prongs inside the patient's nose, as well as general alignment and good functioning of the gas collector and nasal cannula.

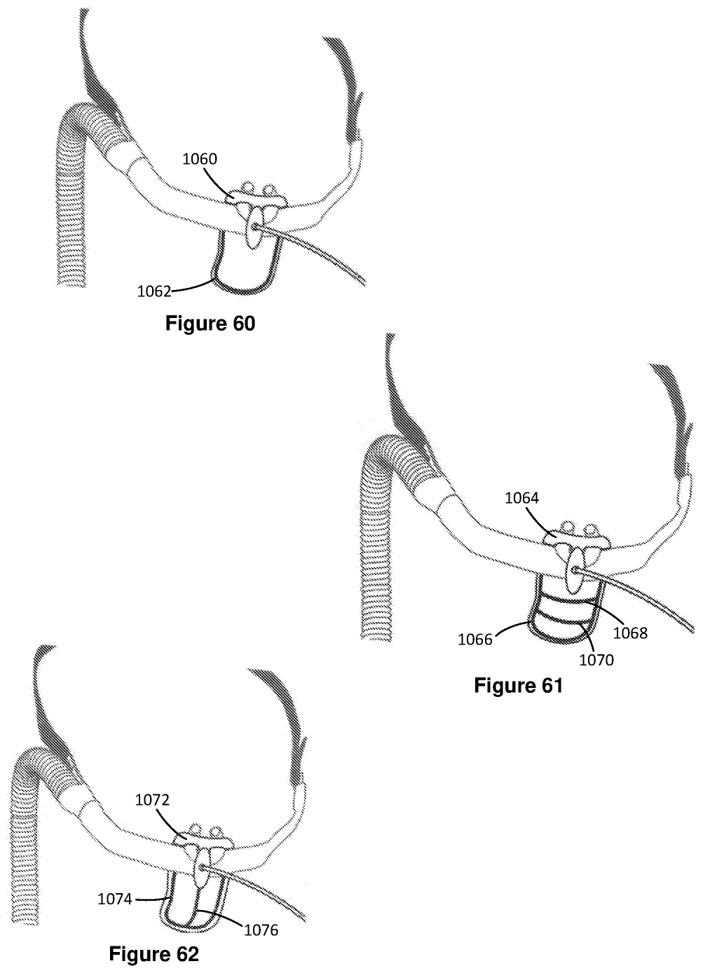

As shown in FIGS. 60 to 62, the body of the gas collector 1040 may be strengthened by integrating structural members in or on the gas collector body. The structural members and may have greater strength in tension and compression relative to the body of the gas collector. In one or more embodiments, the gas collector and the structural members could be of the same material, but the strengthening could be provided by making regions where strengthening is required thicker. In a first embodiment shown in FIG. 60, the body of a gas collector 1060 is strengthened by a structural member 1062 running around the perimeter to ensure that it retains its shape. In a second embodiment shown in FIG. 61, the body of a gas collector 1064 is strengthened by both a structural member 1066 running around the periphery of the body and additionally by horizontal stiffening members 1068 and 1070 to prevent the collapse if the body of the gas collector 1064 when the patient is supine. In some embodiments, the structural member 1062 may not be present. In some embodiments, there may be a plurality of horizontal stiffening members 1068 and 1070 or a single horizontal stiffening member. One or more of the horizontal stiffening members may not extend to the edges of the gas sampler 1040 and may terminate before the edges.

In a further embodiment shown in FIG. 62, the body of the gas collector 1072 includes a peripheral rigid structural member 1074 and in addition a vertical stiffening member 1076 to resist vertical forces applied to the body, so as to prevent collapse of the gas collector 1072 when instruments are inserted into a patient's mouth. In some embodiments, the structural member 1074 may not be present. In some embodiments, a plurality of vertical stiffening members 1076 may be present. One or more of the vertical stiffening member or members may not extend to the edges of the gas sampler 1040 and may terminate before the edges.

It will be appreciated that positioning of the rigid members dictates the direction in which the body of the gas collector in question will be stiff. For example, it may be useful to have the body of a gas collector that can hold its shape when flexed. This can be achieved by embedding malleable members in the body, which means that instead of forces being resisted, given the application of a high enough force, the malleable members (e.g. malleable wires) will flex and the body of the gas collector will hold its shape in the flexed position. This means that the gas collector body can be adjusted to suit different facial contours.

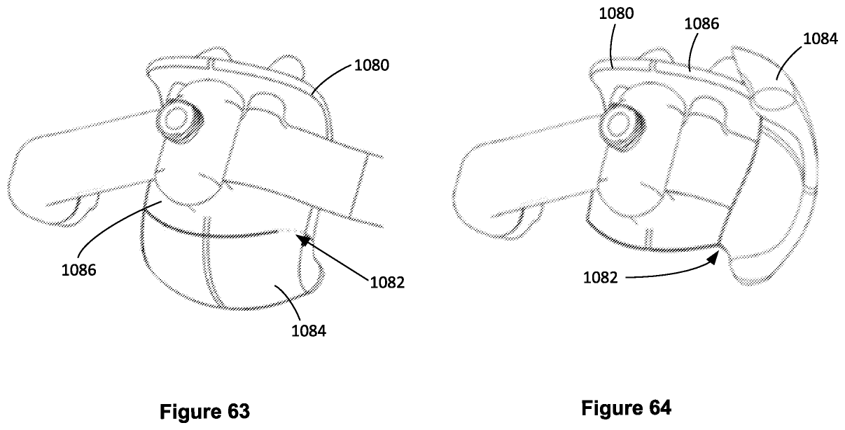

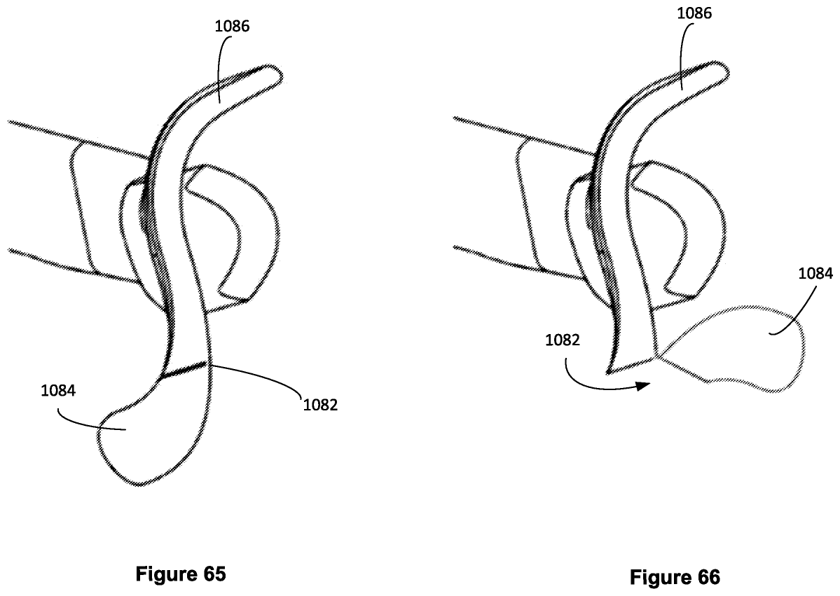

In a further variation to previously described embodiments, shown in FIGS. 63 to 66, it may be beneficial to gain access to the patient's mouth for example, to insert medical equipment. In this case, the body of the gas collector 1080 may include a hinge 1082 to enable a first portion 1084 to be pivotally rotated away from a second portion 1086 of the body of the gas collector 1080. One such method of creating a hinge is by creating a cut out or slit through the body of the gas collector 1080 at an angle. The angle of the slit allows the device to transition between the first position shown in FIGS. 63 and 65 and the second position shown in FIGS. 64 and 66.

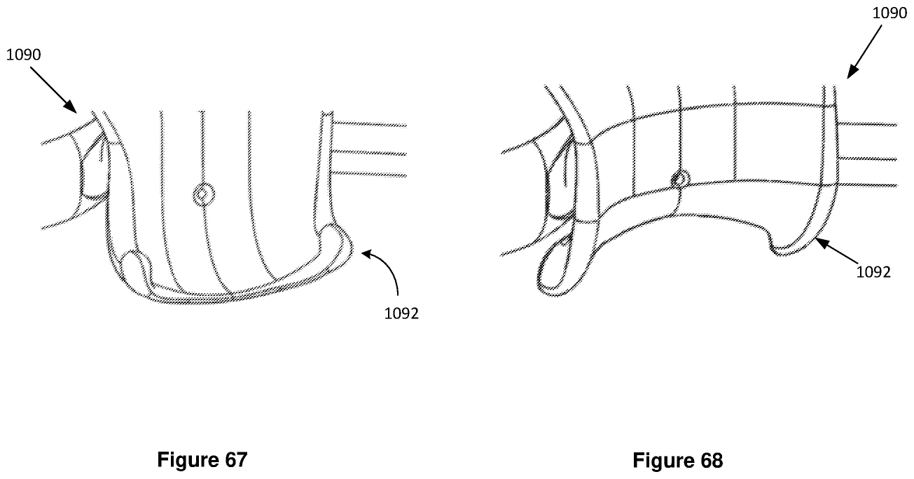

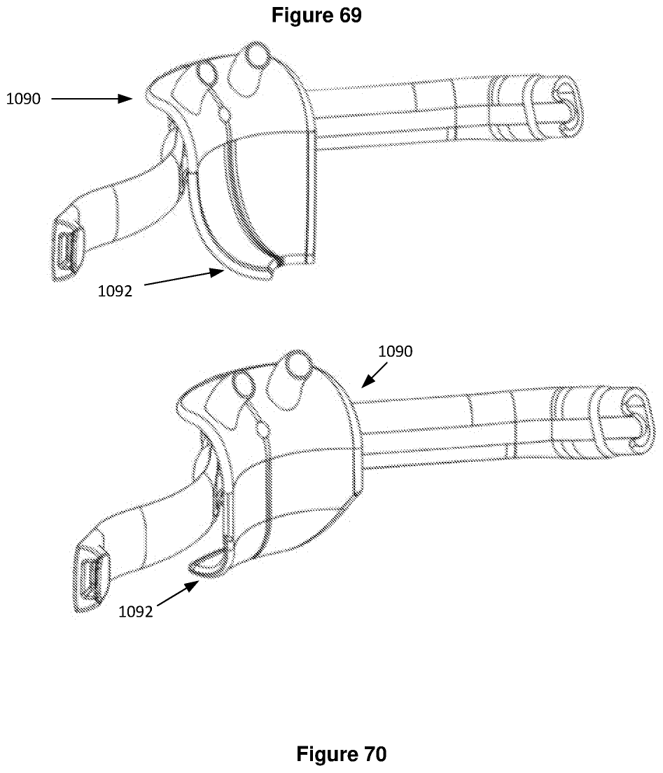

Alternatively, and as depicted in FIGS. 67 to 70, the gas collector body may comprise a bi-stable structure. Concave structures are stable and will hold their shape until a force is applied to them. In the first position shown in FIGS. 67 and 69, the mouth engagement portion 1092 of the gas collector 1090 is used for collecting oral gases. In the second position though, shown in FIGS. 68 and 70, the mouth engagement portion 1092 has been folded and curves away from the patient's mouth to enable the insertion of medical equipment into the patient's mouth. Intermediate positions when switching from the first position to the second position will be unstable and the body of the gas collector 1090 will take the closest stable shape.

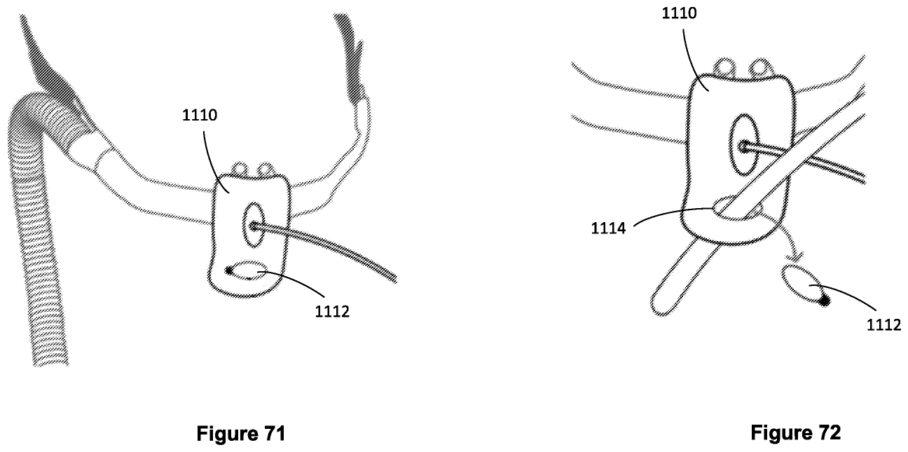

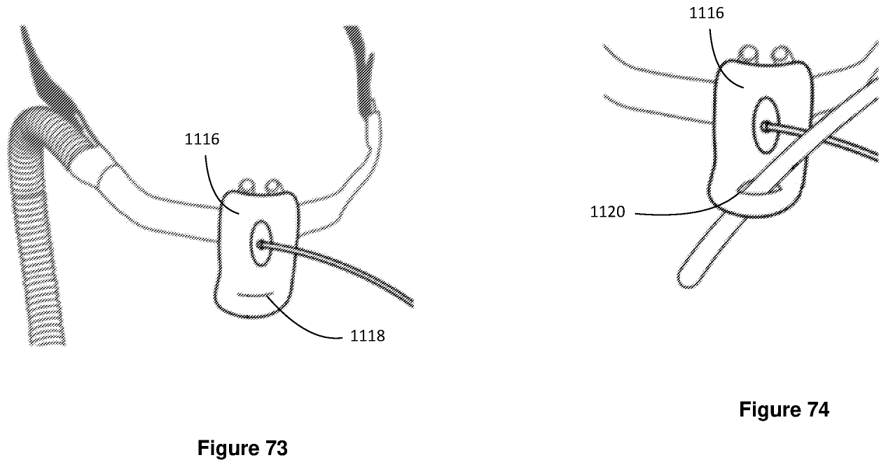

As can be seen in FIGS. 71 to 76, the body of the gas collector may include an opening for the purposes of inserting and retaining medical equipment in the mouth of a patient. The opening may be in the form of a tearaway section that is part of the body of the gas collector, as shown in FIGS. 71 and 72. As can be seen in these figures, the body of a gas collector 1110 may include a tearaway section 1112 which when removed reveals an aperture 1114 through the body of the gas collector 1110 to enable insertion of medical equipment into the mouth of a patient. In an alternative arrangement shown in FIGS. 73 and 74, the body of a gas collector 1116 may include a slit 1118 which, when opened to create an aperture 1120 enables the insertion of that medical equipment into the mouth of a patient. The slit may or may not seal around the medical equipment.

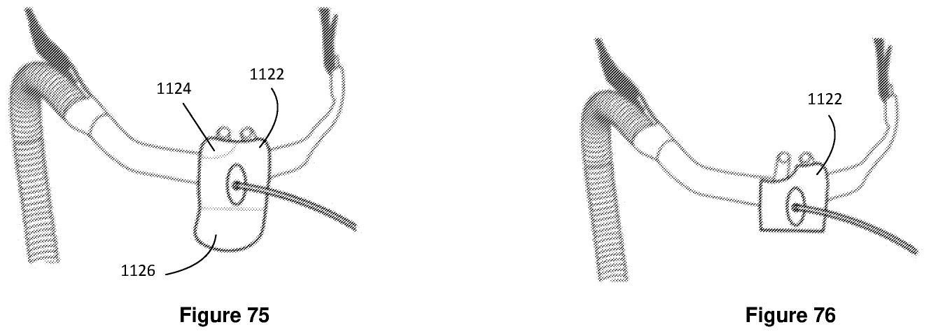

Tearaway sections or section that can be bifurcated, can alternatively or additionally be added to the portion of the gas collector proximate the nasal prongs for delivering gases from the nasal cannula. As shown in FIGS. 75 and 76, the body of a gas collector 1122 can include tearaway sections 1124 and 1126 which are removable and respectively provide access to one of the patient's nasal passages and oral passage providing for insertion of the medical equipment.

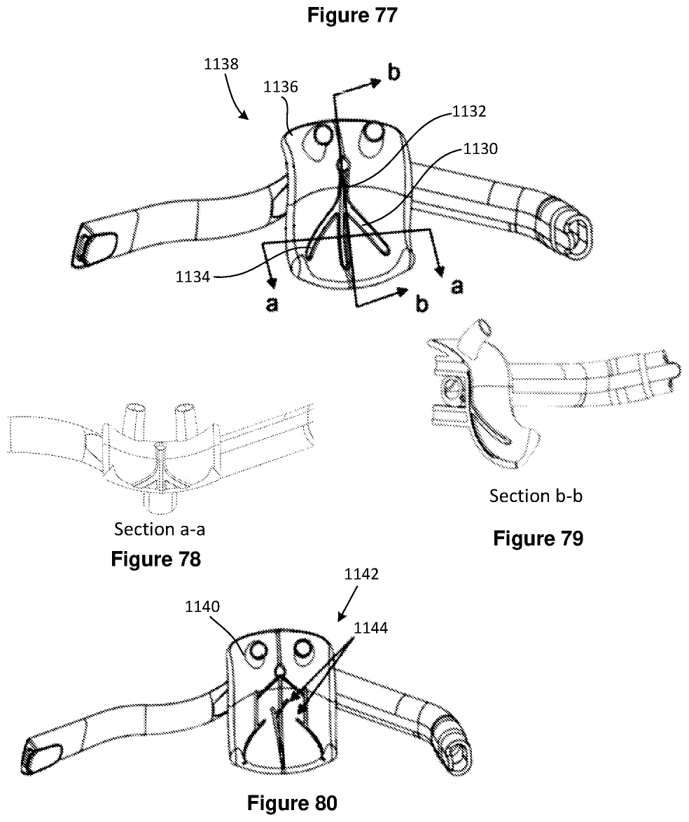

The body of a gas collector may also have grooves on a patient facing side that form smaller open channels. As shown in FIGS. 77 to 80, smaller open channels may be formed within the large open channel formed with the upper lip of a patient. The smaller open channels may be formed in the patient facing side of the body of the gas collector. The grooves are configured to trap patient's bodily fluids such as saliva. Any gases may then travel over the fluids trapped within in the smaller open channels and enter the gas inlet. In FIGS. 77 to 79, three grooves 1130, 1132 and 1134 are provided in the patient facing side 1136 of a gas collector 1138. In some embodiments, there may be a single groove or more than one grooves. The additional grooves-in this exemplary embodiment three grooves are shown-are provided for redundancy. Although one of the grooves runs to a gas collection inlet, this need not be the case in other embodiments.

In FIG. 80, grooves are provided in the patient facing side 1140 of a gas collector 1142. The grooves have some branches that lead to dead ends 1144. Any bodily fluids such as saliva may likely resist changes in flow direction within the grooves and become trapped at the dead ends. In other words, saliva is likely to take the path of least resistance and become trapped in the dead ends. This reduces the chances of the gas collecting inlets being blocked with saliva.

Additionally, or alternatively, the patient facing side 1140 of the gas collector 1142 may be lined with moisture absorbent or hydrophilic materials to reduce the likelihood of the gas collecting inlets becoming blocked.

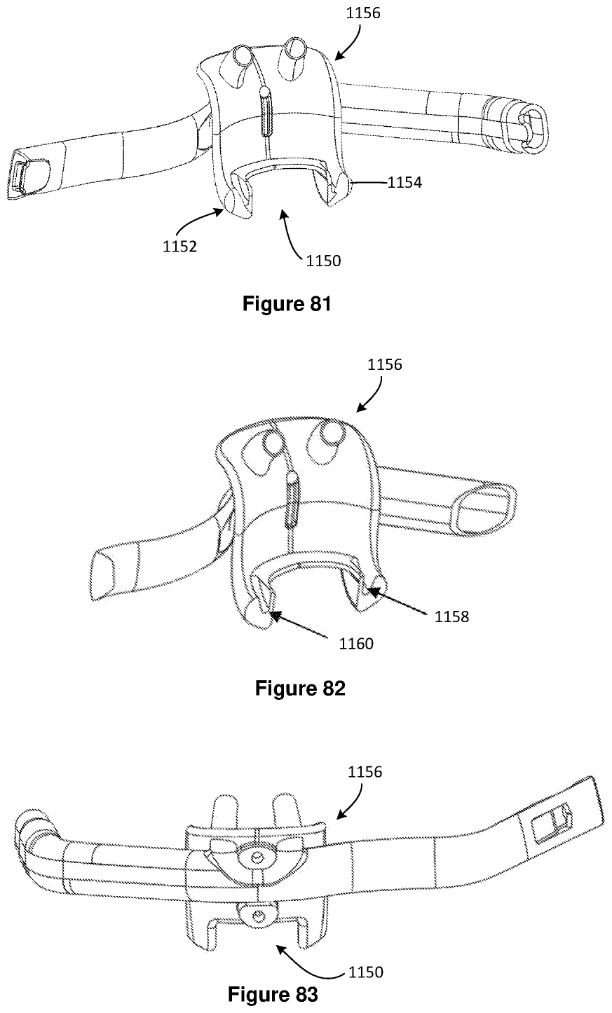

In an arrangement depicted in FIGS. 81 to 83, the abovementioned U-shaped mouth engagement portion could be modified such that a central portion 1150 of the U-shaped section is recessed to sit away from a patient's face. Other shapes for the mouth engagement portion may also be envisaged, such as V-shape. The edges 1152 and 1154 of the body of the gas collector 1156 which extend inside the patient's mouth include channels 1158 and 1160 that engage with the patient's top lip to form two channels that are in fluid communication with the patient's mouth. This creates an open channel between the mouth and the upper lip region of the patient. This avoids covering the patient's upper lip whilst still creating a channel with the upper lip such that the patient can close their mouth in place. The central portion 1150 may permit access to the patient's mouth by a medical instrument without the medical instrument knocking into the gas collector with a force that could impact gas sampling.

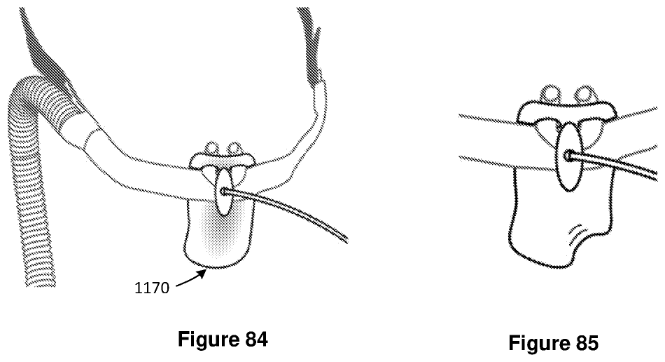

In yet a further variation, and as seen in FIGS. 84 and 85, it may be beneficial to decouple forces transferred between the body of the gas collector and the nasal cannula. This means that when the body of the gas collector is moved or knocked the positioning of the nasal cannula will largely remain unaffected and vice versa. This effect can be achieved by varying the mechanical properties of the gas collector such that the area most prone to knocks or application of forces will absorb the applied forces instead of transferring them to the nasal cannula. Forces may be applied to the outer edge of the gas collector 1170 by the patient's facial features or external equipment. Thus, by making the outer edges flexible the gas collector will absorb some of the force applied when it flexes. This may minimise the magnitude of the force transferred from the sampling device to the nasal cannula. An example of how this can be achieved is by varying the hardness for example, from Shore A 20 to 80 from the outer edge to the inner portion, respectively.

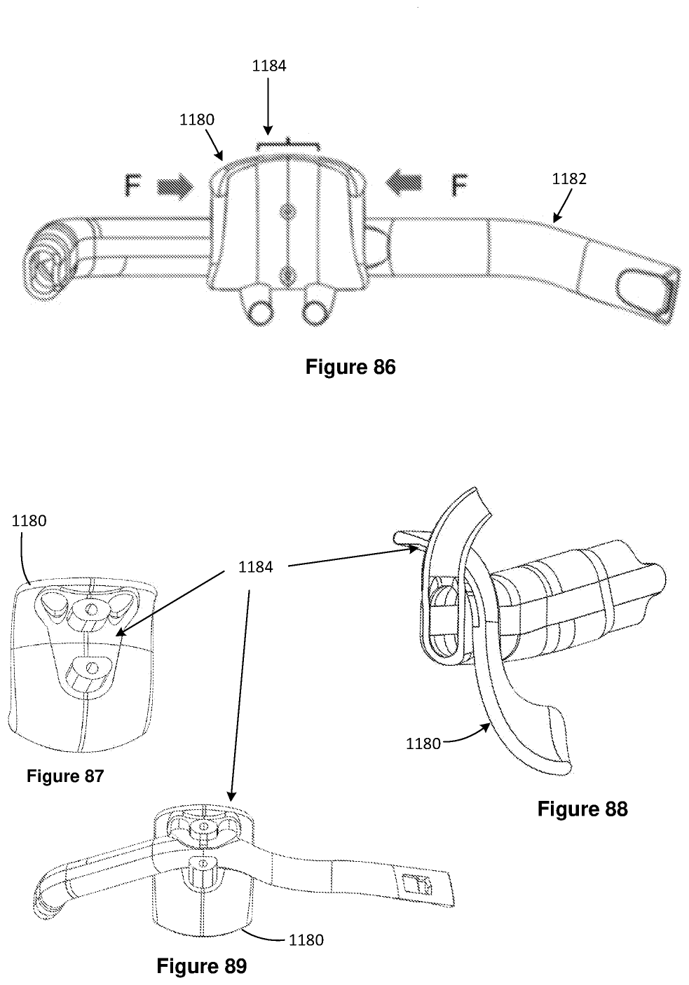

The decoupling effect can also be achieved by making thinner a middle section of the gas collector around the oral gas collecting inlet. The thin material will flex and absorb the force. This will mean less force will be transferred to the nasal cannula. FIGS. 86 to 89 are various representations of a gas collector 1180 coupled to a nasal cannula 1182 and including a thinner middle section 1184. The force applied “F” will cause the thinner middle section 1184 of the gas collector 1180 to deform. Some of this force (energy) is absorbed by the thinner middle section when it deforms. This minimises the magnitude of the force transferred to the nasal canula from the gas collector.

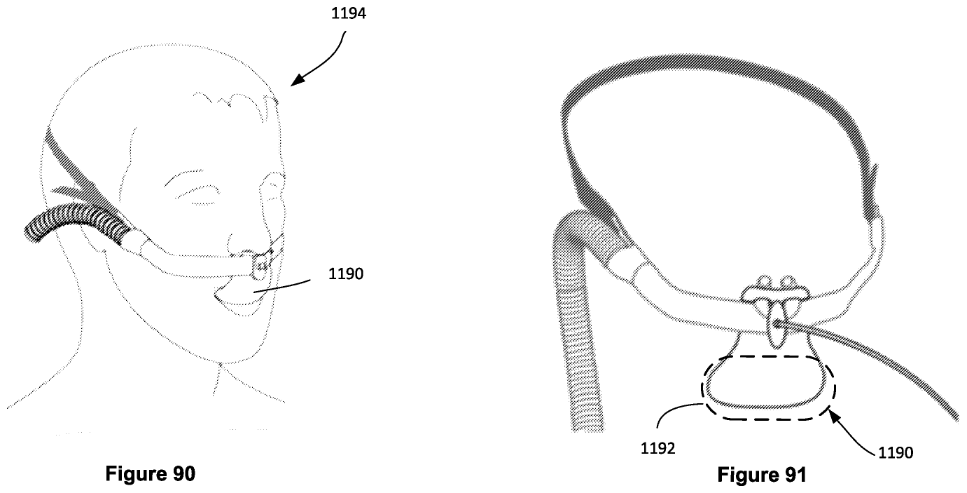

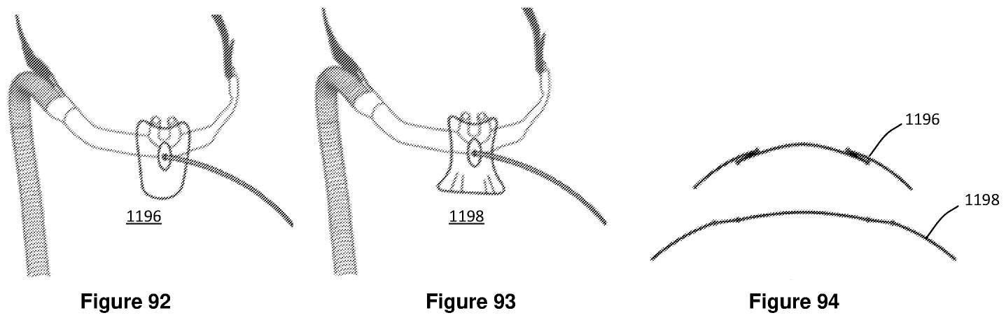

As seen in FIGS. 90 to 97, the mouth engagement portion of the gas collector may have a large mouth engagement portion, and/or the mouth engagement portion may be adjustable to enlarge the mouth engagement portion. The large mouth engagement portion may comprise a lateral dimension that is relative to one or more of a lateral dimension of a nasal cannula (for example the cannula body), a distance between the oral commissures or a lateral dimension of a nose. A gas collector 1190 having a wider than normal mouth engagement portion 1192 is shown in FIGS. 90 and 91 and is adapted to suit a patient 1194 with a wide mouth. The mouth engagement portion may be expandable from a first unexpanded position 1196 shown in FIG. 92 to a second expanded position 1198 shown in FIG. 93. In that regard, the mouth engagement portion may include a series or concertinaed sections, as depicted in FIG. 94. In particular, FIG. 94 illustrates a view of the mouth engagement portion from below in its unexpanded condition 1196 and expanded condition 1198 respectively.

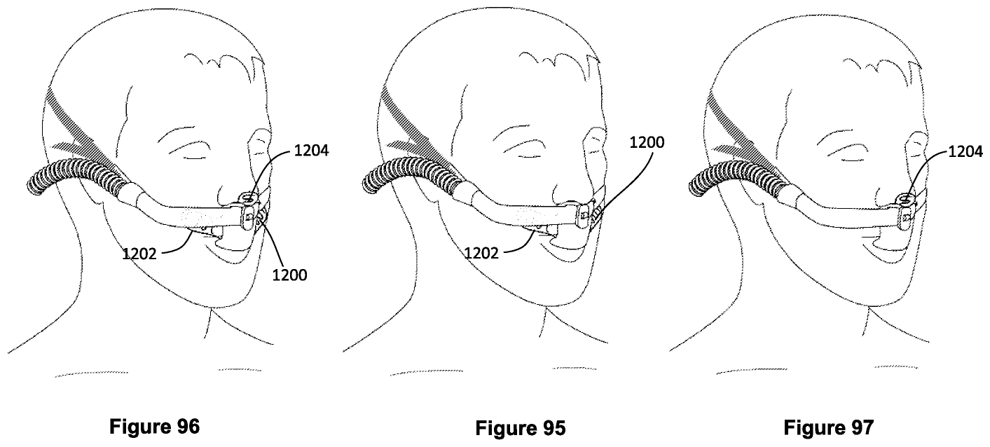

The gas collector may be secured to the patient's face using adhesives. As seen in FIGS. 95 to 97, the adhesive may be laid over flexible tabs 1200 and 1202 that are angled up and away from the patient's mouth region. This is to minimise the effect of movement of the mouth region from dislodging the gas collector and the nasal cannula. Alternatively or additionally, adhesive may be applied to a flexible tab 1204 that is secured to a patient's nose.

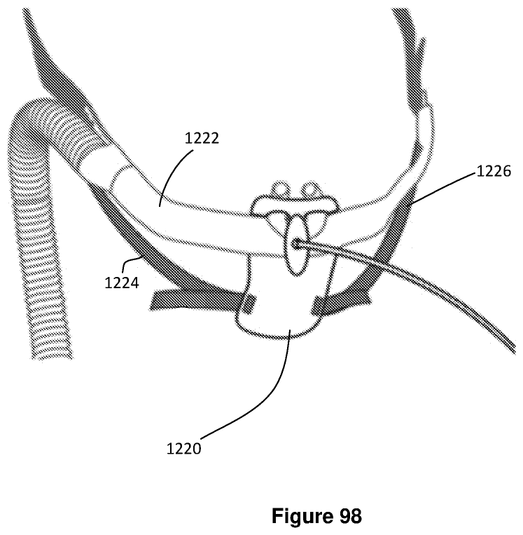

As can be seen in FIGS. 98 to 102, the lower end, or mouth engaging portion, of the gas collector body 1220 may receive additional support by securing it to the nasal cannula 1222 by use of a strap. The strap can keep the body of the gas collector 1220 stable when the patient is moved around or if the body of the gas collector 1220 is accidentally knocked.

As seen in FIG. 98, in one embodiment, straps 1224 and 1226 may be attached between the nasal cannula 1222 and opposing sides of the gas collector 1220 through slots or other attachment mechanisms formed in the gas collector 1220. By tightening one strap and loosening the other, the gas collector 1220 can be manoeuvred to one side or the other of the patient's mouth. This can be useful if medical instruments need to be inserted into the nose or mouth of the patient.

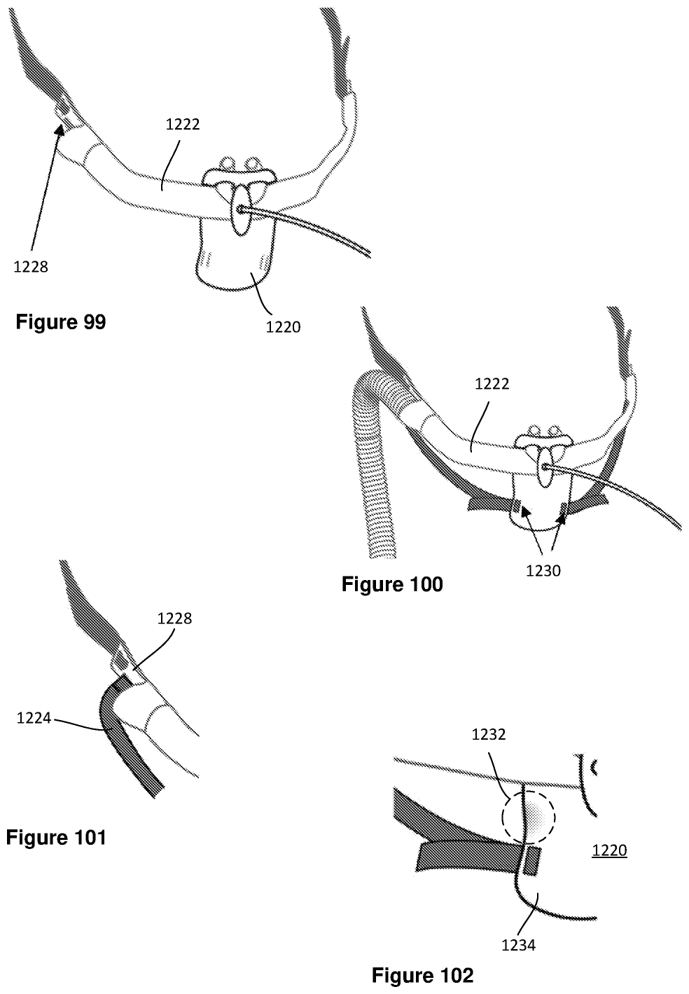

Further details are provided in FIGS. 99 to 102. In FIG. 99, it can be seen that an aperture 1228 may be provided on the nasal cannula 1222 in order to attach a first end of the strap 1224 to the nasal cannula. FIG. 100 depicts apertures 1230 in the body of the gas collector 1220 through which the straps may be attached.

As seen in FIG. 101, each of the straps may be secured at the other end to an aperture in the nasal cannula, and the strap pulled through and attached to itself via stitching or thermal or chemical bond. As shown in FIG. 102, one or more regions of the body of the gas collector 1220, such as the region referenced 1232 adjacent aperture 1234 may be made of thin material such that when the straps are tightened the body of the gas collector 1220 stretches to prevent the nasal canula 1222 from dislodging during the tightening process.

The geometry of the nasal cannulas described in the above-referenced embodiments is curved at the base of the nasal prongs to conform to the patient's upper lip.

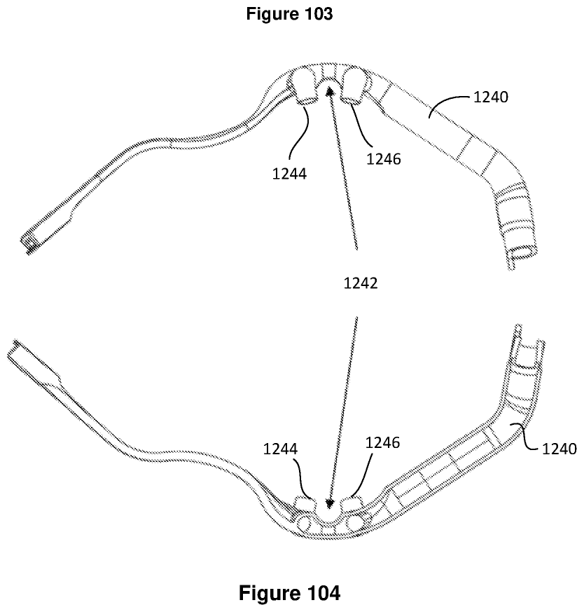

However, in the variation shown in FIGS. 103 and 104, the nasal cannula 1240 has a recess 1242 at the base of the curved nasal prongs 1244 and 1246 to create a channel with the patient's upper lip for the purposes of collecting gases.

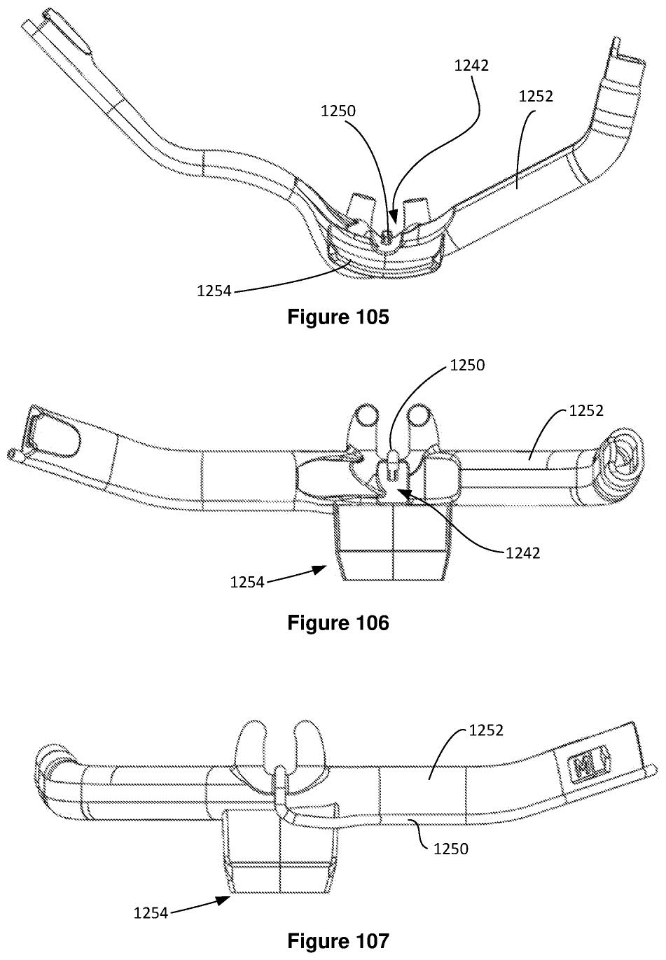

In a still further variation to the above-described embodiments, and as shown in FIGS. 105 to 107, sampling of collected gases may additionally or alternatively be carried out by a small diameter tube 1250 in fluid communication with a channel created with the upper lip of a patient by recess 1242. The small diameter tube 1250 is configured such that an inlet of the tube 1250 is positioned in the formed channel when in use and as more clearly shown in FIG. 106, the inlet is arranged to point inferiorly or towards an oral scoop portion of the gas collector 1254. In such a position, the inlet of the tube 1250 is arranged parallel to a flow in the formed channel and facing away from a gas flow from the patient's nasal region. In other embodiments, the inlet may be arranged differently, for example transverse to a length of the formed channel. The small diameter tube 1250 may be hooked or otherwise connected to the nasal cannula 1252 on which the gas collector 1254 is mounted or integrally formed with.

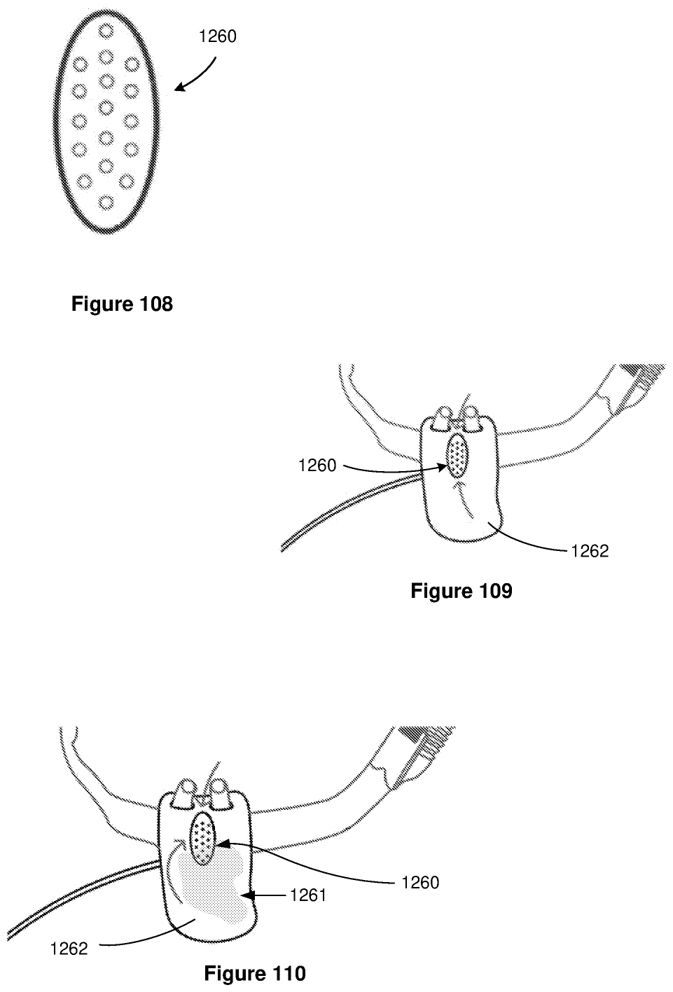

As an alternative to above-described embodiments, the nasal and oral gas collecting inlets could be formed by a plurality of channels 1260 as shown in FIGS. 108 to 110. The plurality of channels 1260 may replace one or both of the nasal and oral gas collecting inlets formed through the body of the gas collector 1262. This plurality of channels provides redundancy in case one or more channels become blocked, for example by material 1261 such as saliva and/or nasal mucus/discharge from the patient as depicted in FIG. 110.

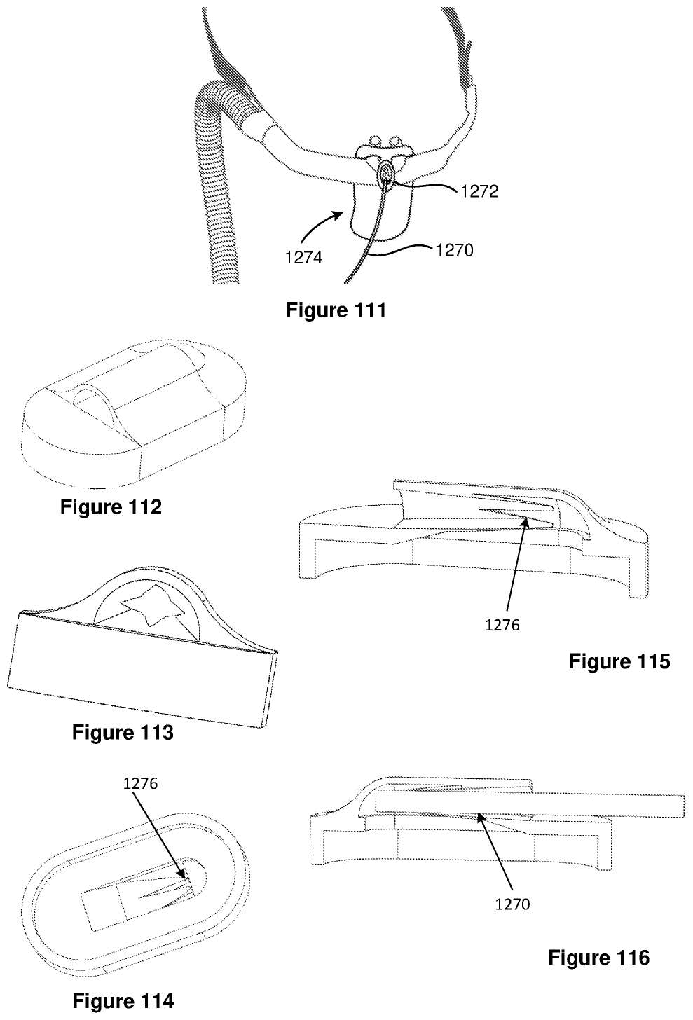

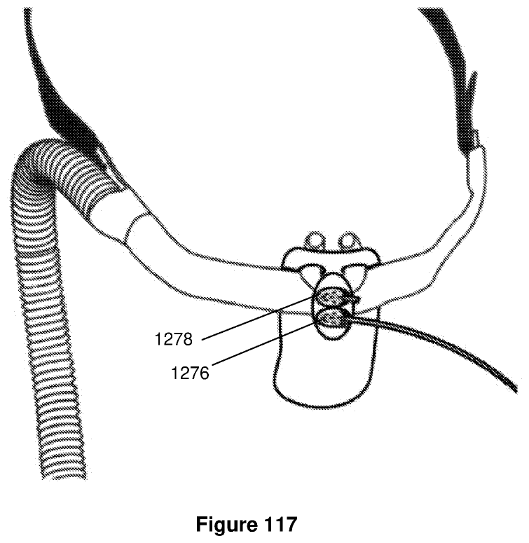

As shown in FIGS. 111 to 123, in yet further variations to the above-described embodiments, it may be convenient to connect a sampling line 1270 to an outlet port 1272 of a gas collector 1274 by a single action, for example, by pushing it to cause connection. A number of single action connection mechanisms are possible. The outlet port 1272 that receives the sampling line 1270 may have multiple tapered members 1276 which flare when the sampling line 1270 is inserted. Friction between the flared thin members and the sampling line will cause the thin members to flex and dig into the sampling line when an attempt is made to pull the sampling line away from the outlet port. Various examples of such tapered members are shown in FIGS. 112 to 116. As depicted in FIG. 117, two or more outlet ports 1276 and 1278 may be provided for redundancy.

Furthermore, the outlet port of the gas collector 1282 may have an extended sampling line connector attached to it. The extended sampling line connector may be integral with or removably attachable to the gas collector. Connectors (e.g. twist and lock mechanism or push in fit type, for example luer connectors) may be used to connect the sampling line (not shown) to the extending sampling line connector. Such an extension allows the connection point to the sampling line to be away from the patient's face. Thus, when attaching the sampling line, forces are not directly applied to the patient's face or the nasal cannula.

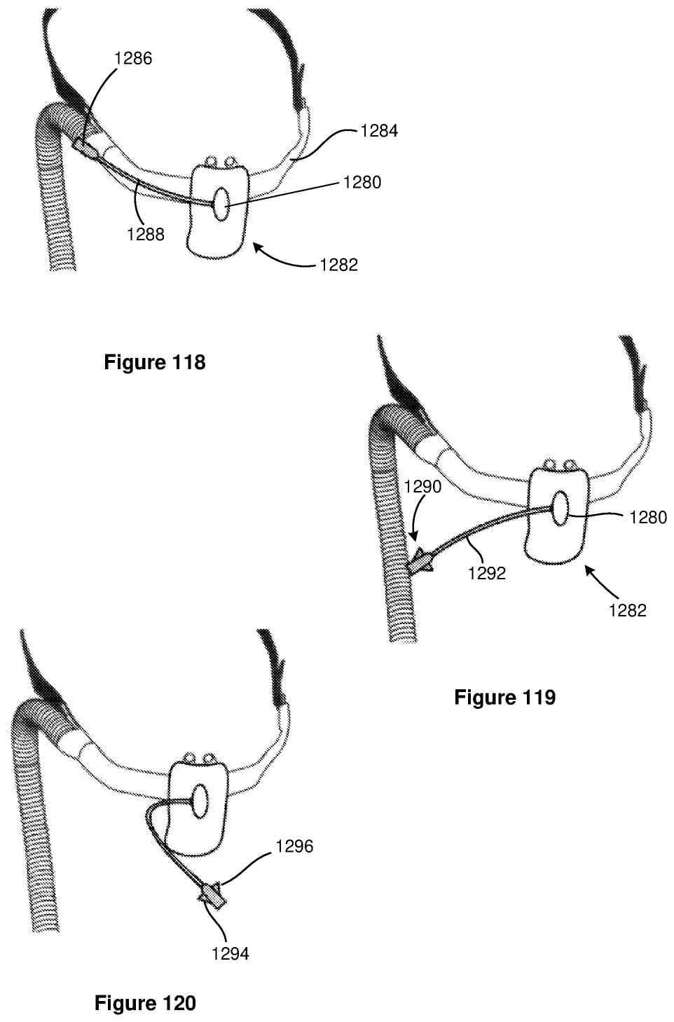

An example is shown in FIG. 118, where an outlet port 1280 forming part of a gas collector 1282 attached to a nasal cannula 1284 is shown. It can be seen that an extended sampling line connector 1286 and tubing 1288 is connected to the outlet port 1280. The extended sampling line and connector may be fixed to a gas path connector of the nasal cannula 1284.

Alternatively, as shown in FIGS. 119 and 120, the extended sampling connector 1290 and line 1292 may be flexible and free to move such that it can be oriented in a variety of ways. Tabs 1294 and 1296 may be provided on the sampling line connector 1290 to assist in gripping the sampling line connector during assembly.

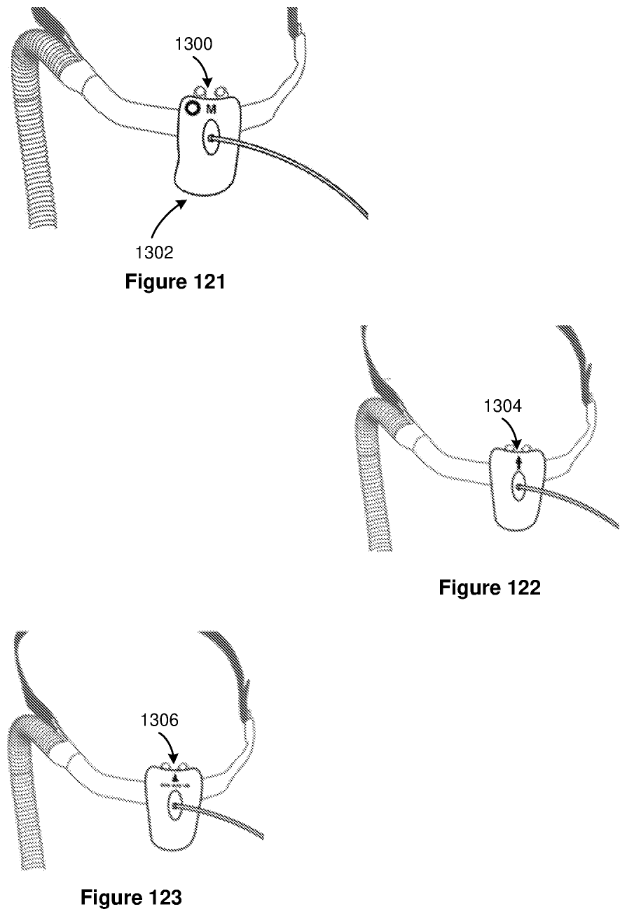

In a still further variation to the above-described embodiments, visual information may be provided to the body of the gas collector to assist in selection of an appropriate device as well as that device's orientation. Representative indicia 1300 indicating the size of the body of the gas collector 1302 is shown in FIG. 121, whereas FIGS. 122 and 123 include exemplary indicia 1304 and 1306, for example, in the form of an arrow 1304 and/or text (e.g. ‘This Way Up’) 1306, to assist in the orientation of the gas collector in use.

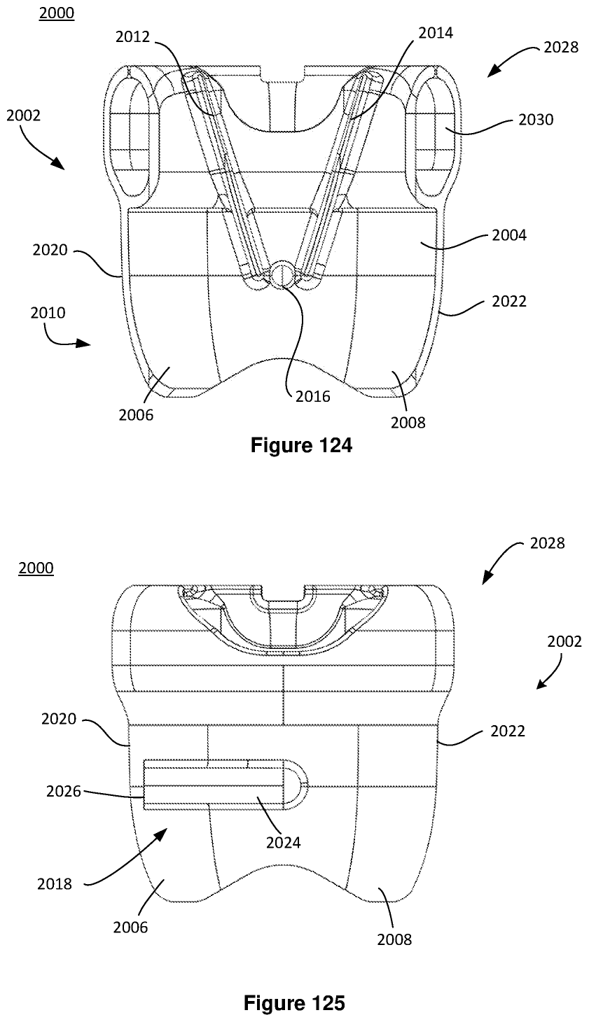

A gas collector 2000 for collecting gases exhaled by a patient from their nasal passages and/or oral passage according to a further embodiment is shown in FIGS. 124 to 127. Similar to some previous embodiments described herein, the gas collector 2000 includes an interface 2002 configured to form a channel at the patient's upper lip. In particular, an inner wall 2004 of the interface 2002 extends generally between the patient's nasal passages and oral passage in use and effectively provides a volume for gathering gasses (e.g. exhaled from the patient's nasal passages and/or oral passage) to be analysed.

The position of the inner wall 2004 may be slightly offset from the patient's face in use. A top portion of the interface 2002 is configured to be positioned below the patient's nasal passages. At a base of the interface 2002, the gas collector 2000 includes a mouth engagement portion 2010 for engaging with the patient's mouth. In particular, the mouth engagement portion 2010 includes a pair of extensions 2006, 2008. Each extension 2006, 2008 being adapted to fit under opposite sides of the patient's upper lip such that the corners of the patient's upper lip are separated from the patient's bottom lip in use to ensure an open oral passage for the patient. As such, the ‘channel’ created by the inner wall 2004 can be considered to have open ends respectively in fluid communication with the patient's nasal passages and oral passage.

The interface 2002 further includes a pair of elongate guide protrusions 2012, 2014. The elongate guide protrusions 2012, 2014 are arranged in a generally V-shaped configuration to more effectively guide (e.g. funnel) gases from the patient's nasal passages to a single sampling inlet 2016. The sampling inlet 2016 may be located generally centrally on the interface 2002. The elongate guiding protrusions 2012, 2014 may also serve as spacers for spacing the inner wall 2004 of the interface 2002 away from the patient's face in use.

As more clearly shown in FIGS. 125 to 127, the gas collector 2000 further includes an outlet 2018 for providing gathered gases to a gas analyser via a conduit (not shown). The outlet 2018 is in fluid communication with the sampling inlet 2016 so that gases collected via the sampling inlet 2016 can be provided to the gas analyser via the outlet 2018.

The interface 2002 includes a pair of opposing lateral sides 2020, 2022. The lateral sides 2020, 2022 may be configured to generally extend in a direction that is aligned with an imaginary vertical plane bisecting the face of the patient.

As more clearly shown in FIGS. 125, 126 and 127, the outlet 2018 defines a receiving port 2024 having an open outlet end 2026 for receiving a portion of the gas analyser conduit therein. The receiving port 2024 of the outlet 2018 is oriented such that its open outlet end 2026 faces towards one of the lateral sides 2020. Advantageously, the receiving port 2024 of the outlet 2018 is configured to allow connection to the conduit from one side of the patient's face.

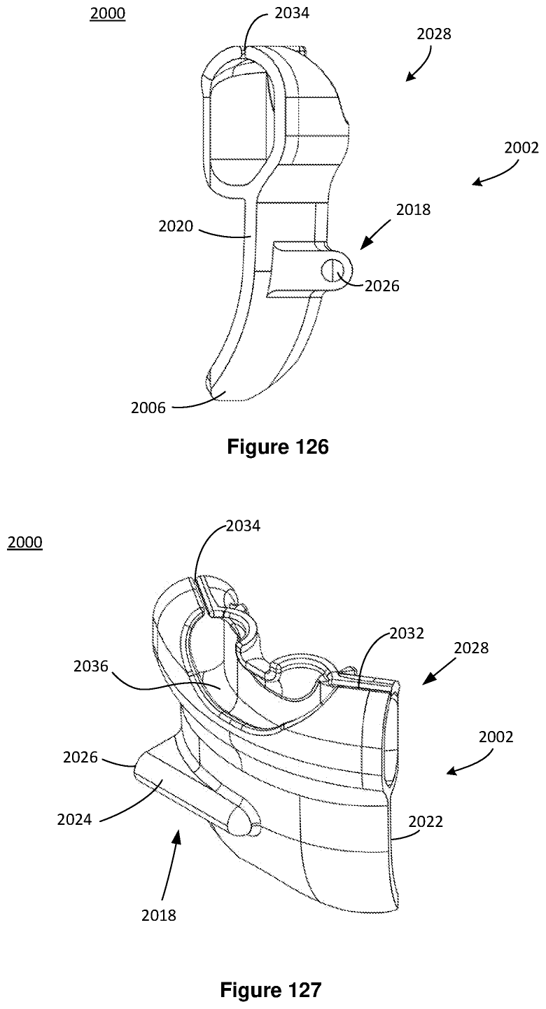

As shown in FIGS. 126 and 127, the receiving port 2024 has a low profile, which advantageously minimises risks of accidental disconnection of the conduit in use.

The gas collector 2000 further includes a mounting portion 2028 for mounting the gas collector 2000 to a nasal cannula (e.g. as shown in FIGS. 5 to 8) for delivering breathable gas to a patient. The mounting portion 2028 defines a sleeve 2030 configured to fit over and receive a portion of the nasal cannula. Internal walls of the sleeve 2030 generally follows an external contour of the portion of the nasal cannula, thereby providing stable mounting of the gas collector 2000 on the nasal cannular in use.

As more clearly shown in FIGS. 126 and 127, the sleeve 2030 defines a pair of slits 2032, 2034 along a top part thereof. The sleeve 2030 further defines an opening 2036 for allowing nasal prongs of the nasal cannula to project therethrough. The slits 2032, 2034 and the opening 2036 allow insertion of a portion of the nasal cannula into the sleeve 2030. In this embodiment, the nasal cannula is securely received in the sleeve 2030 in use. The walls of the nasal cannula may be flexible and can be pressed together to fit through the slits 2032, 2034. In some embodiments, walls of the sleeve 2030 may be resilient so as to allow a width of the slits 2032, 2034 to be manually adjustable, to facilitate mounting to a nasal cannula. The nasal cannula may be mounted to the gas collector 2000 in any suitable way. For example, in other embodiments, any one or more of the previously described features for mounting the nasal cannula to the gas collector may be included in replacement of the mounting portion 2028, in any suitable combination.

As more clearly shown in FIGS. 126 and 127, the gas collector 2000 has a relatively low profile so that the gas collector 2000 is unobstructive in use. This may be particularly beneficial in procedures where clinicians are required to carefully navigate around various medical equipment mounted to the patient's airways in a time critical manner.

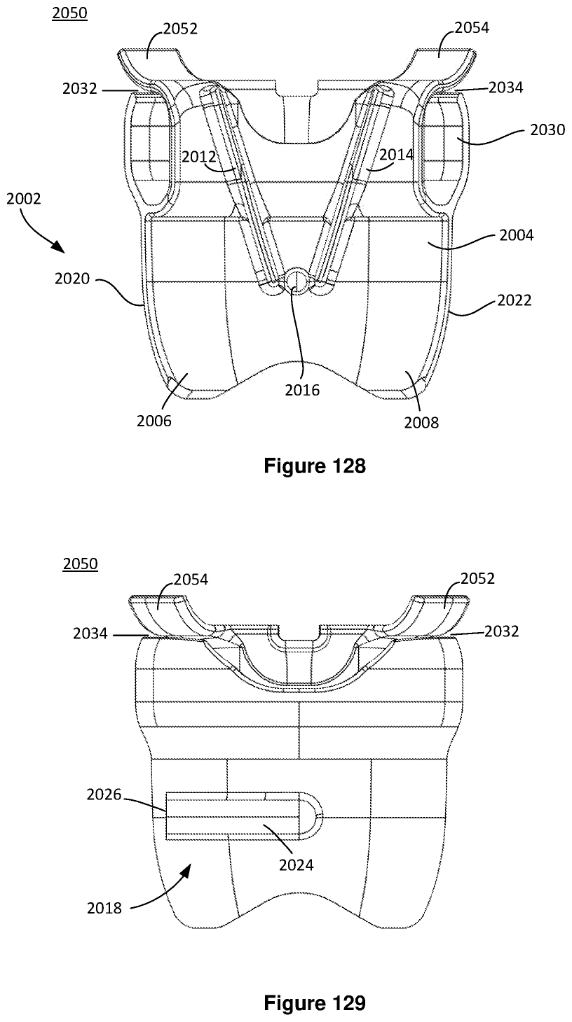

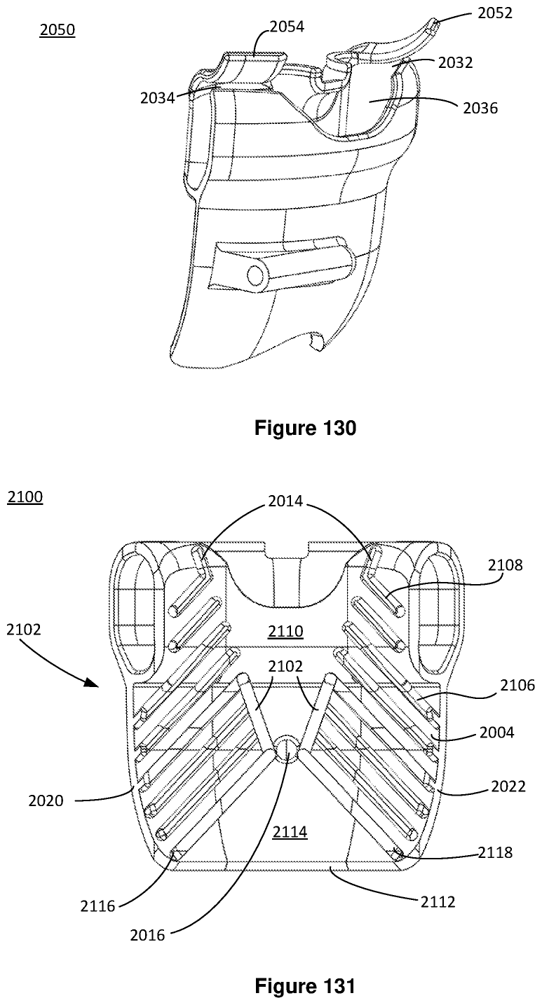

A further gas collector 2050, which is a variation of the gas collector 2000 in FIGS. 124 to 127 will now be described with reference to FIGS. 128 to 130. In FIGS. 128 to 130, like features refer to those previously described.

The gas collector 2050 further includes a pair of nasal guides 2052, 2054. Each nasal guide 2052, 2054 extends outwardly from the inner wall 2004 of the interface 2002 at an upper end of the gas collector 2050. The nasal guides 2052, 2054 serve to guide and facilitate movement of gases from the nasal passages of the patient towards the channel created by the inner wall 2004, and funnelled by elongate guiding protrusions 2012, 2014.

In the gas collector 2050, the slits 2032, 2034 of the sleeve 2030 of the mounting portion 2028 are provided under the nasal guides 2052, 2054. As mentioned, the nasal cannula may be deformed to fit through the slits 2032, 2034 so that it can be received in the sleeve 2030.

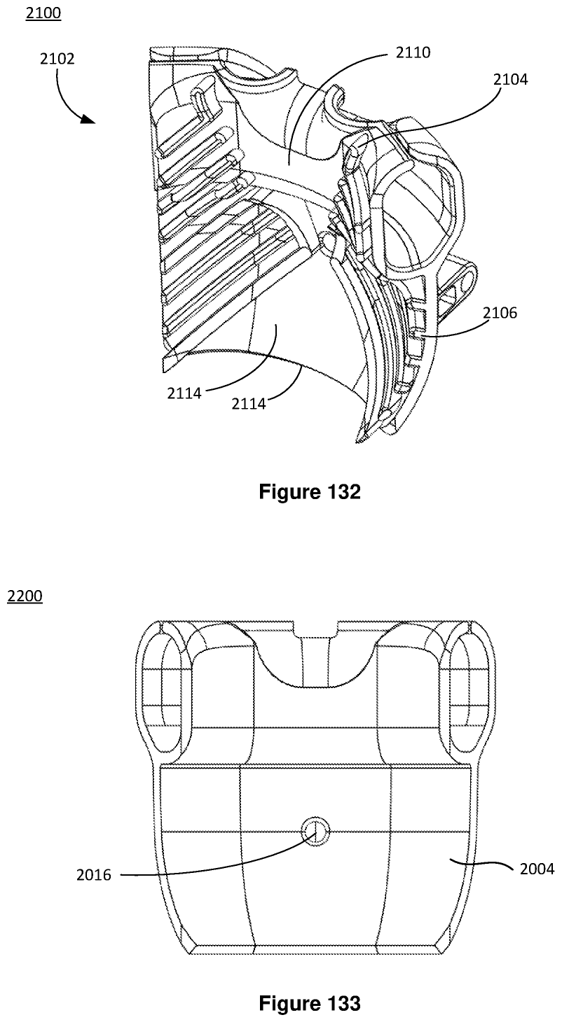

A further gas collector 2100, which is another variation of the gas collector 2000 in FIGS. 124 to 127 will now be described with reference to FIGS. 131 to 132. In FIGS. 131 and 132, like features refer to those previously described.

The gas collector 2100 provides a plurality of ribs 2102 extending from the inner wall 2004 of the interface. A first group of ribs 2102 and/or rib portions 2104 are arranged in a generally V-shaped configuration to funnel gasses from the nasal passages towards the sampling inlet 2016, thereby creating a first generally V-shaped funnel 2110 in an upper central region of the interface. In some embodiments, the ribs 2102 may be longer, such as the elongate guiding protrusions 2012, 2014 shown in FIG. 128. In some embodiments, the ribs 2102 may be shorter.

A second group of ribs 2106 and/or rib portions 2108 extend generally diagonally downwardly from a respective side of the V-shaped funnel 2110 to a corresponding lateral side 2020, 2022 of the inner wall 2004. A bottom pair of ribs 2116, 2118 from the second group of ribs 2106 also form a generally V-shaped configuration to funnel gases from the oral passage towards the sampling inlet 2016, thereby creating a second generally V-shaped funnel 2114 in a lower central region of the interface.

The ribs 2102 advantageously funnel gasses from both the nasal passages and the oral passage of the patient towards the single sampling inlet 2016. (It is envisaged that in some embodiments, as previously described, the ribs 2102 may funnel gasses from the nasal and oral passages towards multiple sampling inlets.) Moreover, the ribs 2102 serve as spacers to create an offset between the patient's face and the inner wall 2004 of the interface in use. In addition, the second group of ribs 2106 and/or rib portions 2108 facilitate draining of liquids from the patient and downwash from the nasal cannula away from the sampling inlet 2016, thereby minimising dilution of exhaled gasses from the nasal passages and oral passage of the patient entering the sampling inlet 2016.

A lower edge 2112 of the interface may extend below the patient's upper lips, or below the patient's lower lips in use. As shown in FIGS. 131 and 132, the lower edge 2112 is generally flat. During use, the lower edge 2112 may be offset and spaced away from the patient's mouth by the ribs 2106. As more clearly shown in FIG. 132, a distance by which the ribs 2106 extend from the inner wall 2004 (also referred to herein as ‘extension distance’) may be varied. For example, portions of the ribs 2106 proximate a lower portion of the interface may extend outwardly further than portions of the ribs configured to sit over the patient's upper lips. This variation in the extension distance of the ribs 2106 may accommodate contours of the patient's lips, whilst maintaining an overall low profile for the gas collector 2100 (e.g. relative to the patient's face in use).

A further gas collector 2200, which is a variation of the gas collector 2100 in FIGS. 131 and 132 will now be described with reference to FIG. 133. In FIG. 133, like features refer to those previously described. In this embodiment, the gas collector 2200 does not include any ribs for funnelling gases from the patient's nasal passages or oral passage. The inner wall 2004 of the interface may be sufficient to effectively channel gasses from the patient's nasal passages and/or oral passage to the sampling inlet 2016.

In some embodiments, a gas collector may generally take the form of the gas collector shown in FIG. 18 as previously described, wherein the outlet 532 may be oriented such that an open end of the outlet 532 faces towards one of the lateral sides of the gas collector similar to that shown in FIGS. 125 to 127.

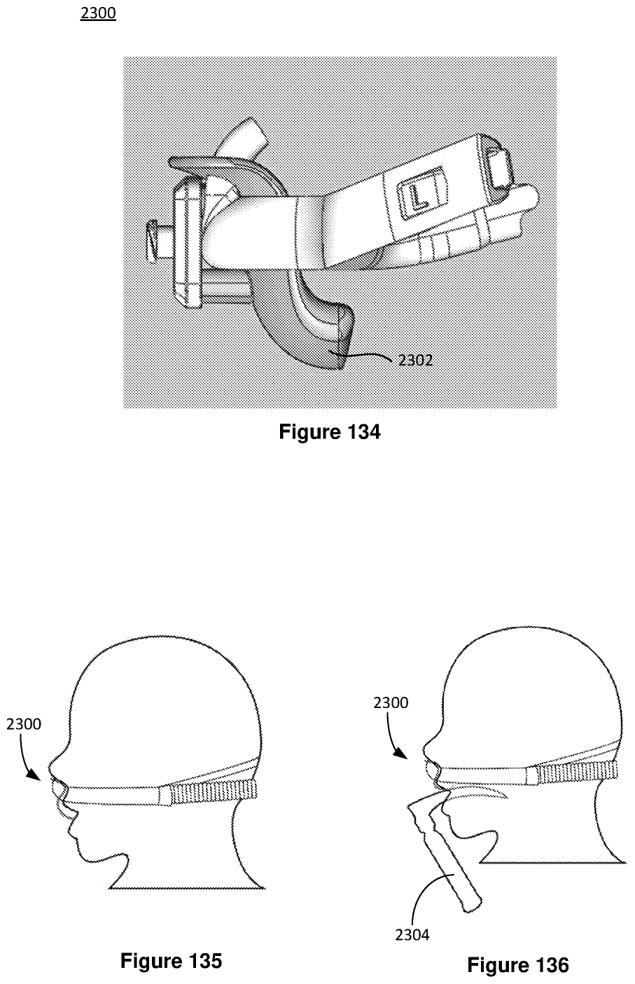

A further gas collector 2300, which is a variation of the first embodiment of the gas collector shown in FIGS. 16 to 20, will now be described with reference to FIGS. 134 to 136. In FIGS. 134 to 136, like features refer to those previously described.

The gas collector 2300 includes a mouth engagement portion 2302 configured to extends further than the first embodiment of the gas collector shown in FIGS. 13 to 20 such that the mouth engagement portion 2302 extends under the front teeth of the patient in use. In some embodiments, the mouth engagement portion 2302 may wrap around the patient's teeth. The mouth engagement portion 2302 can therefore serve as a mouth guard to protect the patient's teeth during medical procedures. For example, as illustrated in FIGS. 135 and 136, in some medical procedures, a laryngoscope 2304 may be used when intubating a patient. In such procedures, there is a risk that the patient's teeth may become chipped due to the force applied by the laryngoscope. The mouth engagement portion 2302 therefore provides protection for the patient's teeth.

Although the present disclosure has been described in terms of certain embodiments, other embodiments apparent to those of ordinary skill in the art are also within the scope of this disclosure. Thus, various changes and modifications may be made without departing from the spirit or scope of the disclosure.

### Interpretation

This specification, including the claims, is intended to be interpreted as follows:

Embodiments or examples described in the specification are intended to be illustrative of the invention, without limiting the scope thereof. The invention is capable of being practised with various modifications and additions as will readily occur to those skilled in the art. Accordingly, it is to be understood that the scope of the invention is not to be limited to the exact construction and operation described or illustrated, but only by the following claims.

The mere disclosure of a method step or product element in the specification should not be construed as being essential to the invention claimed herein, except where it is either expressly stated to be so or expressly recited in a claim.

The terms in the claims have the broadest scope of meaning they would have been given by a person of ordinary skill in the art as of the relevant date.

The terms “a” and “an” mean “one or more”, unless expressly specified otherwise.

Neither the title nor the abstract of the present application is to be taken as limiting in any way as the scope of the claimed invention.

Where the preamble of a claim recites a purpose, benefit or possible use of the claimed invention, it does not limit the claimed invention to having only that purpose, benefit or possible use.

It should be noted that terms of degree such as “generally”, “substantially”, “about” and “approximately” as used herein mean a reasonable amount of deviation of the modified term such that the end result is not significantly changed. These terms of degree should be construed as including a deviation of the modified term if this deviation would not negate the meaning of the term it modifies.

In the specification, including the claims, the term “comprise”, and variants of that term such as “comprises” or “comprising”, are used to mean “including but not limited to”, unless expressly specified otherwise, or unless in the context or usage an exclusive interpretation of the term is required.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made if the end result is not significantly changed.

As used herein, the wording “and/or” is intended to represent an inclusive-or. That is, “X and/or Y” is intended to mean X or Y or both, for example. As a further example, “X, Y, and/or Z”is intended to mean X or Y or Z or any combination thereof.

Throughout the specification, like reference numerals refer to like features described herein. As such, any instance where features or components are indicated with the same references implies a direct correlation to the similar or identical features or components as previously described in the specification.

The disclosure of any document referred to herein is incorporated by reference into this patent application as part of the present disclosure, but only for purposes of written description and enablement and should in no way be used to limit, define, or otherwise construe any term of the present application where the present application, without such incorporation by reference, would not have failed to provide an ascertainable meaning. Any incorporation by reference does not, in and of itself, constitute any endorsement or ratification of any statement, opinion or argument contained in any incorporated document.

1. A gas collector for collecting gases at a patient, the gas collector including:
an interface configured to form at least one channel at an upper lip of the patient, the channel being in fluid communication with the patient's nose and mouth regions,
wherein the interface includes one or more spacers configured to contact the patient's face so as to space a channel wall portion from the patient's face.

2. A gas collector according to claim 1, wherein the interface further includes the channel wall portion.

3. A gas collector according to either one of claims 1 or 2, wherein interface is configured to be attached to or integral with a nasal cannula.

4. A gas collector according to claim 3, wherein the nasal cannula at least partially forms the channel wall portion.

5. A gas collector according to either one of claims 3 or 4, wherein the nasal cannula includes nasal prongs for delivering gas to the patient's nasal passages, the gas collector further including one or more openings formed through the channel wall portion for receiving the nasal prongs.

6. A gas collector according to any one of the preceding claims, wherein the channel is partly formed by the patient's upper lip.

7. A gas collector according to any one of the preceding claims, wherein the spacers are disposed to contact the patient's face on either side of the patient's upper lip.

8. A gas collector according to any one of the preceding claims, wherein, in use, the spacers extend from the channel wall portion to the patient's face.

9. A gas collector according claim 8, wherein the spacers are integral with the channel wall portion.

10. A gas collector according either one of claims 8 or 9, wherein the spacers have a thickness greater than the channel wall portion.

11. A gas collector according any one of claim 8 to 10, wherein the spacers are more rigid that the channel wall portion.

12. A gas collector according to claim 11, wherein the spacers have a greater Young's modulus than the channel wall portion.

13. A gas collector according to any one of the preceding claims, wherein the channel wall portion is configured to enable it to flex when in contact with the patient's face.

14. A gas collector according to any one of the preceding claims, and further including
an interface forming a gas collection area and including a first and second gas collecting inlets in fluid communication with the gas collection area.

15. A gas collector according to claim 14, and further including
a mouth engagement portion configured to project into the patient's mouth so as to maintain an open passage between the patient's mouth and the second gas collecting inlet.

16. A gas collector according to claim 15, wherein the mouth engagement portion includes a lower end projecting into the patient's mouth, the lower end of the mouth engagement portion being shaped to maintain an open passage between the patient's mouth and the second gas collecting inlet.

17. A gas collector according to claim 16, wherein the lower end of the mouth engagement portion has an inner surface which includes
edge portions located, when in use, at the sides of the patient's mouth, and a central portion located, when in use, at the centre of the patient's mouth and spaced further from the patient's upper lip than the edge portions.

18. A gas collector according to claim 17, wherein the inner surface of a first end of the mouth engagement portion has a U or V shaped profile.

19. A gas collector according to any one of the preceding claims, wherein the channel wall portion includes an upper end extending outwardly from the patient's face when in use.

20. A gas collector according to claim 15, wherein the upper end of the channel wall curves outwardly from the patient's face when in use.

21. A gas collector for collecting gases exhaled by a patient from their nasal passages and oral passage, the gas collector including
an interface is configured to form a channel at the patient's upper lip, the channel having open ends respectively in fluid communication with the patient's nasal passages and oral passage,
the channel providing a volume for gathering gases to be analysed.

22. A gas collector according to claim 21, and further including
one or more gas collecting conduits, each having a gas collecting inlet, configured to deliver gases from the channel to a localised gas collector area, and
an outlet for providing gathered gases from the localised gas collector area to a gas analyser.

23. A gas collector according to claim 22, wherein the channel is partly formed by the patient's upper lip.

24. A gas collector according to any one of claims 21 to 23, and further including a gas flow diverter configured to funnel at least some of the gases exhaled by a patient to one or more of the gas sampling inlets.

25. A gas collector according to claim 24, wherein the interface includes one or more spacers for contacting the patient's face to space a channel wall portion from the patient's face, and wherein the gas flow diverter is integrated with the channel wall portion.

26. A gas collector according to claim 25, wherein the sampler body includes one or more spacers for contacting the patient's face to space a channel wall portion from the patient's face, and wherein the gas flow diverter is formed separately from the channel wall portion.

27. A gas collector according to either one of claims 25 or 26 wherein one or more of the gas sampling inlets form nasal gas sampling inlets located proximate the patient's nasal passages.

28. A gas collector according to claim 27, wherein the one or more nasal gas sampling inlets are formed in the channel wall portion.

29. A gas collector according to claim 27, wherein the one or more nasal gas sampling inlets are formed in the spacers.

30. A gas collector according to any one of claims 21 to 29, wherein one or the gas sampling inlets forms an oral gas sampling inlet located proximate the patient's oral passage.

31. A gas collector according to claim 30, wherein the oral gas sampling inlet is formed in the channel wall portion.

32. A gas collector for collecting gases at a patient, the gas collector including
an interface forming a gas collection area and including at least one nasal gas collecting inlet and an oral gas collecting inlet in fluid communication with the gas collection area, and
a mouth engagement portion configured to project into the patient's mouth so as to maintain an open passage between the patient's mouth and the oral gas collecting inlet.

33. A gas collector for collecting gases at a patient, the gas collector including
an interface configured to form a channel at an upper lip of the patient,
one or more gas sampling conduits, each having a gas sampling inlet, for drawing gases from the channel to a localised gas collector area, and
a gas flow diverter configured to direct at least some of the gases exhaled by a patient to one or more of the gas sampling inlets, wherein
the gas flow diverter includes
an upper portion that extends away from the patient's face.

34. A gas collector according to anyone of claims 1 to 20, wherein
the spacer, in use, rests against the patient's upper lip; and wherein
the gas collector further includes
one or more nasal gas collecting conduits formed within the spacer, each nasal gas collecting conduit having a nasal gas collecting inlet in fluid communication with the patient's nasal passages, for delivering gases exhaled by a patient from their nasal passages to an outlet;
an oral gas collecting conduit formed within the spacer, the oral gas collecting conduit having an oral gas collecting inlet in fluid communication with the patient's oral passage; and
a gas flow diverter configured to direct at least some of the gases exhaled by a patient from their oral passage to the oral pas collecting inlet.

35. A gas collector for sampling gases at a patient, the gas collector including
one or more nasal gas collecting conduits, each nasal gas collecting conduit having a nasal gas collecting inlet in fluid communication with the patient's nasal passages; and
an oral gas collecting conduit, the oral gas collecting conduit having an oral gas collecting inlet in fluid communication with the patient's oral passage and the outlet,
wherein the one or more nasal gas sampling conduits and the oral gas sampling conduit form a junction at localised gas collector area, and
wherein the one or more nasal gas sampling conduits and the oral gas sampling conduit are configured so that a flow rate in the one or more nasal gas sampling conduits is a percentage of a total flow rate in the one or more nasal gas sampling conduits and the oral gas sampling conduit, where the percentage is within a predetermined range.

36. A patient interface including:
a nasal cannula for delivering breathable gas to a patient; and
a gas collector according to any one of the preceding claims, the gas collector removably attachable to or integrated with the nasal cannula.

37. A gas collector of claim 21, further including an outlet for providing gathered gases to a gas analyser via a conduit, the outlet being configured to allow connection to the conduit from one side of the patient's face.

38. The gas collector of claim 37, wherein the interface has a pair of opposing lateral sides, and
wherein the outlet defines a receiving port having an open outlet end for receiving a portion of the conduit therein, the receiving port being oriented such that the open outlet end faces towards one of the lateral sides.

39. The gas collector of any one of claims 21, 37 and 38, the outlet being a single outlet of the gas collector to provide gathered gases to the gas analyser.

40. The gas collector of any one of claims 37 to 39, further including a single gas sampling inlet configured to provide gathered gases from the channel to the outlet.

41. The gas collector of any one of claims 21, and 37 to 40, further including a mounting portion for mounting the gas collector to a nasal cannula, the nasal cannula for delivering breathable gas to a patient, the mounting portion defining a sleeve configured to fit over a portion of the nasal cannula.

42. The gas collector of claim 41, wherein the sleeve generally follows an external contour of the portion of the nasal cannula.

43. The gas collector of claim 41 or 42, wherein sleeve defines one or more slits, the one or more slits allowing insertion of the portion of the nasal cannula therethrough so as to enable the portion of the nasal cannula to be received in the sleeve.

44. The gas collector of claim 43, wherein the sleeve has a resilient wall so as to allow a width of the one or more slits to be manually adjustable.
